(19) United States
(12) Patent Application Publication
(10) Pub. No.: US 2025/0234910 A1
(43) Pub. Date: Jul. 24, 2025

(54) FRAGRANCE AND FLAVOR COMPOSITIONS COMPRISING THIENYL ALKANOATE DERIVATIVES

(71) Applicant: OSMO LABS, PBC, Cambridge, MA (US)

(72) Inventors: Alexander WILTSCHKO, Somerville, MA (US); Christophe LAUDAMIEL, New York, NY (US); Bradley B. GILBERT, Milford, NJ (US); Andrew P. PATRON, Oceanside, CA (US); Benjamin AMORELLI, Brielle, NJ (US)

(21) Appl. No.: 19/036,929

(22) Filed: Jan. 24, 2025

Related U.S. Application Data
(60) Provisional application No. 63/624,742, filed on Jan. 24, 2024.

(51) Int. Cl. A23L 27/20; C07D 333/16; C07D 333/24

(52) U.S. Cl. CPC A23L 27/2052; C07D 333/16; C07D 333/24

(57) ABSTRACT

The present application relates to thienyl alkanoates and related compounds, methods of making them, and methods of using them as flavor and fragrance ingredients in food, cosmetic, pharmaceutical, consumer, and other compositions and products.

### CROSS-REFERENCE TO RELATED APPLICATIONS

This application is an application filed under 35 U.S.C. § 111(a) which claims priority to, and the benefit of, U.S. Provisional Applic. Ser. No. 63/624,742, filed on Jan. 24, 2024, the contents of which are hereby incorporated by reference in its entirety.

### FIELD OF THE APPLICATION

The present application relates to thienyl alkanoates and related compounds, methods of making them, and methods of using them as flavor and fragrance ingredients in food, cosmetic, pharmaceutical, consumer, and other compositions and products.

### BACKGROUND

Scent is an important factor used to produce a sense of anticipation, quality, palatability, and security to many consumer products. Flavor is particularly important for foodstuffs. Identifying effective aromas and flavors to impart in a product is an element that contributes to the success of the product, and is useful in product marketing, consumer satisfaction, and consumer retention. Toasted coconut and other nutty smells may be particularly desirable for certain flavors and fragrances and may be used in toiletries, cosmetics, household cleaners, room sprays, laundry, and fine fragrance applications, such as in perfumes and toilet water, dental hygiene products (such as toothpastes and mouthwashes), orally administered medications, and food products.

Considerable work is performed by many scientists relating to identifying new substances which can be used, alone or in combinations, to impart to, or enhance, the aroma or flavor of various consumable materials, including, e.g., cosmetics, cleaners, and foodstuffs. While there may be some trends in the relationship between chemical structure and flavor or fragrance—such as common use of low molecular weight aldehydes and alcohols as flavors and fragrances—the precise aroma associated with a molecule is exceedingly difficult to predict. Small changes in structure, such a lengthening or shortening a functional group by just one carbon atom, can have profound and unexpected effects on a compound's flavor or fragrance profile. The art of flavor and fragrance prediction is still in its infancy.

Using a proprietary predictive method, the inventors have identified thienyl alkanoates and related compounds, as new ingredients useful in flavors and fragrances. Such compounds have not been specifically identified as a flavor or fragrance ingredient, nor have their smells or odors been previously described.

This application describes the surprising and unexpected olfactive qualities of thienyl alkanoates, and analogs and derivatives thereof, and their use as fragrance and flavor ingredients, and potential applications thereof.

### BRIEF SUMMARY

In one aspect, the application relates to compounds of Formula IA, IB, IC, ID, IE, and IF (collectively “Formula I”):

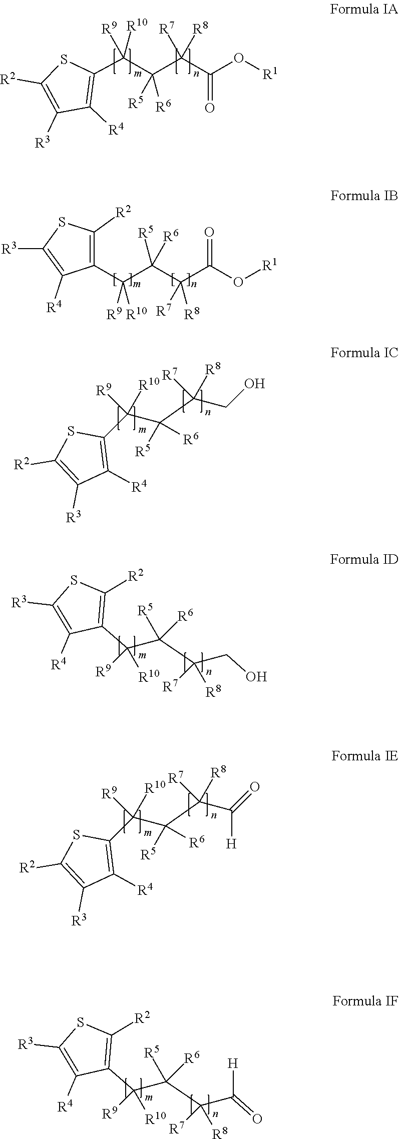

wherein:
R¹ is C₁₋₄alkyl (e.g., methyl, ethyl, n-propyl, isopropyl, n-butyl, isobutyl, sec-butyl or tert-butyl), aryl (e.g., phenyl), arylC₁₋₄alkyl (e.g., benzyl), C₃₋₆cycloalkyl (e.g., cyclopropyl), C₃₋₆cycloalkyl-C₁₋₄alkyl (e.g., cyclopropylmethyl), or C₂₋₄alkenyl (e.g., propenyl or butenyl);
R², R³, and R⁴ are each independently selected from H, C₁₋₃alkyl (e.g., methyl, ethyl, n-propyl, isopropyl), C₁₋₃alkoxy (e.g., methoxy), and C₂₋₄alkenyl (e.g., propenyl);
R⁵, R⁶, R⁷, and R⁸ are each independently selected from OH, H, C₁₋₃alkyl (e.g., methyl), and C₃₋₆cycloalkyl (e.g., cyclopropyl, cyclobutyl, cyclopentyl or cyclohexyl); or R⁵ and R⁶ or R⁷ and R⁸, independently, together with the carbon atom to which they are attached, form a C₃₋₄cycloalkyl ring (e.g., a spiro-fused cyclopropyl); or
R⁹ and R¹⁰ are both H, or R⁹ and R¹⁰ together form a C(═O) group (carbonyl group), or R⁹ is OH and R¹⁰ is H or C₁₋₃alkyl (e.g., methyl);
or R⁵ and R⁹ together with the carbon atoms to which they are attached form a C₃₋₆cycloalkyl (e.g., cyclopropyl, cyclobutyl, and R⁶ and R¹⁰ are each independently selected from OH, H, and C₁₋₃alkyl (e.g., methyl); or R⁵ and R⁷ together with the carbon atoms to which they are attached form a C₃₋₆cycloalkyl (e.g., cyclopropyl, cyclobutyl, and R⁶ and R⁸ are each independently selected from OH, H, and C₁₋₃alkyl (e.g., methyl); and
m and n are independently 0 or 1,
optionally, provided that when m is 0, n is 0 or 1, and R¹ is C₁₋₄alkyl, aryl (e.g., phenyl), or C₃₋₆cycloalkyl (e.g., cyclopropyl), then (i) at least one of R²-R⁸ is not H or C₁₋₃alkyl, or (ii) R²-R⁸ are not all H or C₁₋₃alkyl; and/or
provided that when m is 0, n is 0, and R¹ is C₂₋₄alkenyl (e.g., ethenyl), then (i) at least one of R²-R⁶ is not H or C₁₋₃alkyl, or (ii) R²-R⁶ are not all H or C₁₋₃alkyl;
and optionally provided that the compound is not ethyl 3-(3-thienyl)propionate.

In some embodiments, the compound of Formula I is ethyl 3-(3-thienyl)propionate.

In another embodiment of the first aspect, the application relates to compounds of Formula IIA, IIB, IIC, IID, IIE, and IIF (collectively “Formula II”):

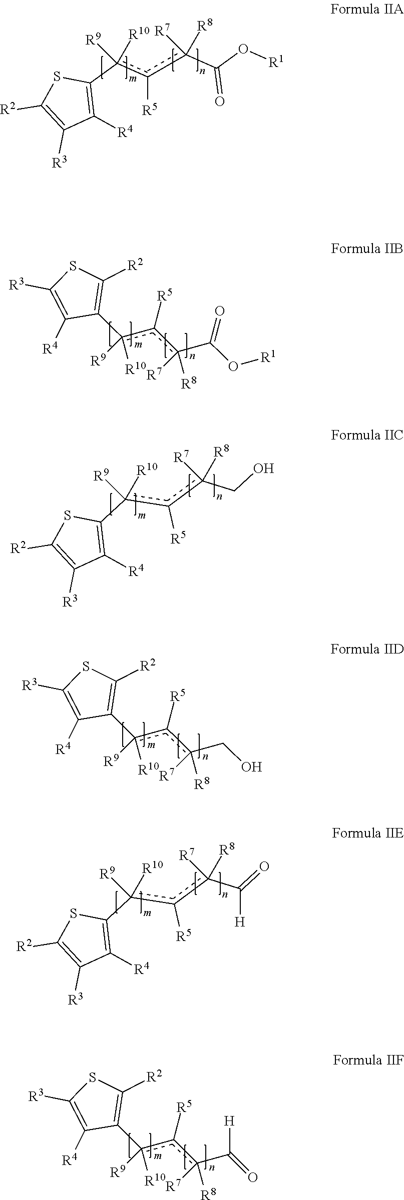

wherein
[symbol] means either a single bond or a double bond, provided one of the [symbol] is a single bond and the other [symbol] is a double bond;
R¹ is C₁₋₄alkyl (e.g., methyl, ethyl, n-propyl, isopropyl, n-butyl, isobutyl, sec-butyl or tert-butyl), aryl (e.g., phenyl), arylC₁₋₄alkyl (e.g., benzyl), C₃₋₆cycloalkyl (e.g., cyclopropyl), C₃₋₆cycloalkyl-C₁₋₄alkyl (e.g., cyclopropylmethyl), or C₂₋₄alkenyl (e.g., propenyl or butenyl); R², R³, and R⁴ are each independently selected from H, C₁₋₃alkyl (e.g., methyl, ethyl, n-propyl, isopropyl), C₁₋₃alkoxy (e.g., methoxy), and C₂₋₄alkenyl (e.g., propenyl); R⁵, R⁷, and R⁸ are each independently selected from OH, H, C₁₋₃alkyl (e.g., methyl), and C₃₋₆cycloalkyl (e.g., cyclopropyl, cyclobutyl, cyclopentyl or cyclohexyl); or R⁷ and R⁸, together with the carbon atom to which they are attached, form a C₃₋₄cycloalkyl ring (e.g., a spiro-fused cyclopropyl), provided that R⁸ is absent when R⁷ is attached to a double-bonded carbon atom; or
R⁹ and R¹⁰ are both H, or R⁹ and R¹⁰ together form a C(═O) group (carbonyl group), or R⁹ is OH and R¹⁰ is H or C₁₋₃alkyl (e.g., methyl), provided that R⁹ is absent when R¹⁰ is attached to a double-bonded carbon atom; and
m and n are independently 0 or 1,
optionally, provided that when m is 0, n is 0 or 1, and R¹ is C₁₋₄alkyl, aryl (e.g., phenyl), or C₃₋₆cycloalkyl (e.g., cyclopropyl), then (i) at least one of R²-R⁸ is not H or C₁₋₃alkyl, or (ii) R²-R⁸ are not all H or C₁₋₃alkyl; and/or
optionally provided that when m is 0, n is 0, and R¹ is C₂₋₄alkenyl (e.g., ethenyl), then (i) at least one of R²-R⁶ is not H or C₁₋₃alkyl, or (ii) R²-R⁶ are not all H or C₁₋₃alkyl.

In another aspect, the application relates to fragrance and flavor compositions comprising the compound of Formula I (e.g., Formula IA, IB, IC, ID, IE, or IF) and/or the compound of Formula II (e.g., Formula IIA, IIB, IIC, IID, IIE, or IIF), optionally comprising one or more additives, additional fragrance or flavor ingredients, or a combination of additives and fragrance or flavor ingredients. In some embodiments, the application relates to fragrance and flavor compositions comprising ethyl 3-(3-thienyl)propionate.

In another aspect, the application relates to products, such as consumer products, comprising such fragrance and flavor compositions comprising the compound of Formula I (e.g., Formula IA, IB, IC, ID, IE, or IF) and/or the compound of Formula II (e.g., Formula IIA, IIB, IIC, IID, IIE, or IIF), as herein provided, e.g., products comprising ethyl 3-(3-thienyl)propionate.

In another aspect, the present disclosure provides a method of making compounds of Formula I or the compounds of Formula II.

The details of one or more embodiments of the application are set forth in the accompanying description below. Unless defined otherwise, all technical and scientific terms used herein have the same meaning as commonly understood by one of ordinary skill in the art to which this application belongs. In the case of conflict, the present specification will control.

Other features and advantages of the application will be apparent from the following detailed description, examples, and claims.

### DETAILED DESCRIPTION

The inventors have unexpectedly found that the compound ethyl 3-(3-thienyl)propionate has a unique and favorable aroma. It has thus been determined to be useful in imparting and providing desirable aromas and/or flavors to the products to which it is added. Other Compounds of Formula I (e.g., Formula IA, IB, IC, ID, IE, or IF) are expected to likewise have pleasant or desirable flavors and/or aromas. Such compounds are therefore potentially useful for products where the inclusion of a pleasing fragrance or flavor is desired, including, but not limited to, perfumes, household products, laundry products, personal care products, cosmetics, dental hygiene products, orally administered medications, and food products. The Compounds of Formula I (e.g., Formula IA, IB, IC, ID, IE, or IF) may be employed in varying amounts depending upon the specific fragrance or flavor product application, the nature and amount of other flavor or fragrance ingredients present, and the desired aroma and/or flavor of the product.

In a first aspect, the present disclosure provides a compound of Formula I wherein the compound is a compound of Formula IA, IB, IC, ID, IE, and IF:

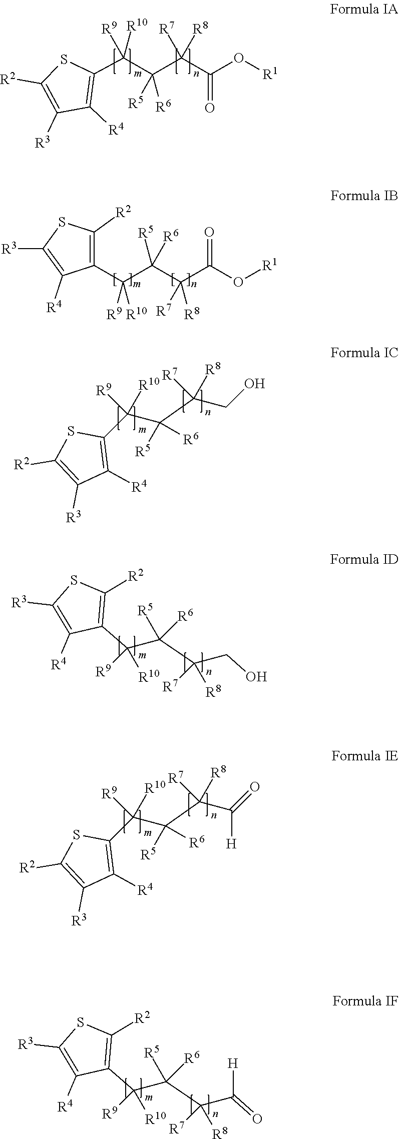

wherein:
R¹ is C₁₋₄alkyl (e.g., methyl, ethyl, n-propyl, isopropyl, n-butyl, isobutyl, sec-butyl or tert-butyl), aryl (e.g., phenyl), arylC₁₋₄alkyl (e.g., benzyl), C₃₋₆cycloalkyl (e.g., cyclopropyl), C₃₋₆cycloalkyl-C₁₋₄alkyl (e.g., cyclopropylmethyl), or C₂₋₄alkenyl (e.g., propenyl or butenyl);
R², R³, and R⁴ are independently selected from H, C₁₋₃alkyl (e.g., methyl, ethyl, n-propyl, isopropyl), C₁₋₃alkoxy (e.g., methoxy), and C₂₋₄alkenyl (e.g., propenyl);
R⁵, R⁶, R⁷, and R⁸ are each independently selected from OH, H, C₁₋₃alkyl (e.g., methyl), and C₃₋₆cycloalkyl (e.g., cyclopropyl, cyclobutyl, cyclopentyl or cyclohexyl); or R⁵ and R⁶ or R⁷ and R⁸, independently, together with the carbon atom to which they are attached, form a C₃₋₄cycloalkyl ring (e.g., spiro-fused cyclopropyl);
R⁹ and R¹⁰ are both H, or R⁹ and R¹⁰ together a C(═O) group (carbonyl group) or R⁹ is OH and R¹⁰ is H or C₁₋₃alkyl (e.g., methyl);
or R⁵ and R⁹ together with the carbon atoms to which they are attached form a C₃₋₆cycloalkyl (e.g., cyclopropyl, cyclobutyl, and R⁶ and R¹⁰ are each independently selected from OH, H, and C₁₋₃alkyl (e.g., methyl); or R⁵ and R⁷ together with the carbon atoms to which they are attached form a C₃₋₆cycloalkyl (e.g., cyclopropyl, cyclobutyl, and R⁶ and R⁸ are each independently selected from OH, H, and C₁₋₃alkyl (e.g., methyl); and
m and n are independently 0 or 1,
optionally, provided that when m is 0, n is 0 or 1, and R¹ is C₁₋₄alkyl, aryl (e.g., phenyl), or C₃₋₆cycloalkyl (e.g., cyclopropyl), then (i) at least one of R²-R⁸ is not H or C₁₋₃alkyl, or (ii) R²-R⁸ are not all H or C₁₋₃alkyl; and/or provided that when m is 0, n is 0, and R¹ is C₂₋₄alkenyl (e.g., ethenyl), then (i) at least one of R²-R⁶ is not H or C₁₋₃alkyl, or (ii) R²-R⁶ are not all H or C₁₋₃alkyl; and optionally provided that the compound is not ethyl 3-(3-thienyl)propionate.

In further embodiments of the first aspect, the present disclosure provides:
1.1 Compound of Formula I, wherein R¹ C₁₋₄alkyl (e.g., methyl, ethyl, n-propyl, isopropyl, n-butyl, isobutyl, sec-butyl or tert-butyl), aryl (e.g., phenyl), arylC₁₋₄alkyl (e.g., benzyl), C₃₋₆cycloalkyl (e.g., cyclopropyl), C₃₋₆cycloalkyl-C₁₋₄alkyl (e.g., cyclopropylmethyl), or C₂₋₄alkenyl (e.g., propenyl or butenyl);
1.2 Compound of Formula I, wherein R¹ is C₁₋₄alkyl (e.g., methyl, ethyl, n-propyl, isopropyl, n-butyl, isobutyl, sec-butyl or tert-butyl);
1.3 Compound of Formula I, wherein R¹ is aryl (e.g., phenyl);
1.4 Compound of Formula I, wherein R¹ is arylC₁₋₄alkyl (e.g., benzyl);
1.5 Compound of Formula I, wherein R¹ is C₃₋₆cycloalkyl (e.g., cyclopropyl);
1.6 Compound of Formula I, wherein R¹ is C₃₋₆cycloalkyl-C₁₋₄alkyl (e.g., cyclopropylmethyl);
1.7 Compound of Formula I, wherein R¹ is C₂₋₄alkenyl (e.g., propenyl or butenyl);
1.8 Compound of Formula I, or any of 1.1-1.7, wherein R², R³, and R⁴ are each independently H, C₁₋₃alkyl (e.g., methyl, ethyl, n-propyl, iso-propyl), C₁₋₃alkoxy (e.g., methoxy), or C₂₋₄alkenyl (e.g., propenyl);
1.9 Compound of Formula I, or any of 1.1-1.7, wherein R², R³, and R⁴ are each H;
1.10 Compound of Formula I, or any of 1.1-1.7, wherein R², R³, and R⁴ are each independently C₁₋₃alkyl (e.g., methyl, ethyl, n-propyl, isopropyl);
1.11 Compound of Formula I, or any of 1.1-1.7, wherein R², R³, and R⁴ are each independently C₁₋₃alkoxy (e.g., methoxy);
1.12 Compound of Formula I, or any of 1.1-1.7, wherein R², R³, and R⁴ are each independently C₂₋₄alkenyl (e.g., propenyl);
1.13 Compound of Formula I, or any of 1.1-1.12, wherein R⁵, R⁶, R⁷, and R⁸ are independently OH, H, C₁₋₃alkyl (e.g., methyl), or C₃₋₆cycloalkyl (e.g., cyclopropyl, cyclobutyl, cyclopentyl or cyclohexyl), or R⁵ and R⁶, or R⁷ and R⁸, independently, together with the carbon atom to which they are attached, form a C₃₋₄cycloalkyl ring (e.g., spiro-fused cyclopropyl);
1.14 Compound of Formula I, or any of 1.1-1.12, wherein R⁵, R⁶, R⁷, and R⁸ are independently H;
1.15 Compound of Formula I, or any of 1.1-1.12, wherein R⁵, R⁶, R⁷, and R⁸ are independently C₁₋₃alkyl (e.g., methyl);
1.16 Compound of Formula I, or any of 1.1-1.12, wherein R⁵, R⁶, R⁷, and R⁸ are independently C₃₋₆cycloalkyl (e.g., cyclopropyl, cyclobutyl, cyclopentyl or cyclohexyl);
1.17 Compound of Formula I, or any of 1.1-1.12, wherein R⁵ and R⁶, or R⁷ and R⁸, independently, together with the carbon atom to which they are attached, form a C₃₋₄cycloalkyl ring (e.g., spiro-fused cyclopropyl);
1.18 Compound of Formula I, or any of 1.1-1.12, wherein R⁵ is H and R⁶ is OH;
1.19 Compound of Formula I, or any of 1.1-1.12, wherein R⁷ is H and R⁸ is OH;
1.20 Compound of Formula I, or any of 1.1-1.17, wherein R⁹ and R¹⁰ are H, or R⁹ and R¹⁰ together form a C(═O) group;
1.21 Compound of Formula I, or any of 1.1-1.17, wherein R⁹ and R¹⁰ are H;
1.22 Compound of Formula I, or any of 1.1-1.17, wherein R⁹ and R¹⁰ together form a C(═O) group;
1.23 Compound of Formula I, or any of 1.1-1.17, wherein R⁹ is OH and R¹⁰ is H;
1.24 Compound of Formula I, or any of 1.1-1.17, wherein R⁵ and R⁹ together with the carbon atoms to which they are attached form a C₃₋₆cycloalkyl (e.g., cyclopropyl, cyclobutyl, and R⁶ and R¹⁰ are each independently selected from OH, H, and C₁₋₃alkyl (e.g., methyl);
1.25 Compound of Formula I, or any of 1.1-1.17, wherein R⁵ and R⁷ together with the carbon atoms to which they are attached form a C₃₋₆cycloalkyl (e.g., cyclopropyl, cyclobutyl, and R⁶ and R⁸ are each independently selected from OH, H, and C₁₋₃alkyl (e.g., methyl);
1.26 Compound of Formula I, or any of 1.1-1.22, wherein m is 0 or 1;
1.27 Compound of Formula I, or any of 1.1-1.22, wherein m is 0;
1.28 Compound of Formula I, or any of 1.1-1.22, wherein m is 1;
1.29 Compound of Formula I, or any of 1.1-1.28, wherein n is 0 or 1;
1.30 Compound of Formula I, or any of 1.1-1.28, wherein n is 0;
1.31 Compound of Formula I, or any of 1.1-1.28, wherein n is 1;
1.32 Compound of Formula I, or any of 1.1-1.31, wherein the compound of Formula I is the compound of Formula IA, and wherein R¹-R¹⁰ and m and n are defined as in any of the preceding formulae;
1.33 Compound of Formula I, or any of 1.1-1.31, wherein the compound of Formula I is a compound selected from the following:

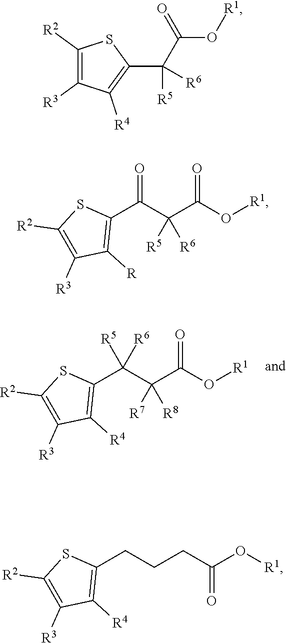

wherein R¹-R⁸ are defined as in any of the preceding formulae;
1.34 Compound of Formula I, or any of 1.1-1.31, wherein the compound of Formula I is the compound of Formula IB, and R¹-R¹⁰ and m and n are as defined in any of the preceding formulae;
1.35 Compound of Formula I, or any of 1.1-1.31, wherein the compound of Formula I is a compound selected from the following:

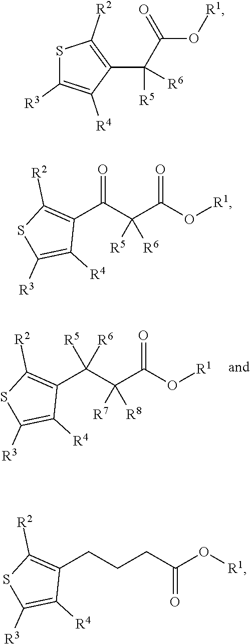

wherein R¹-R⁸ are as defined in any of the preceding formulae;
1.36 Compound of Formula I, or any of 1.1-1.31, wherein the compound of Formula I is a compound of Formula IA or IB selected from the following:

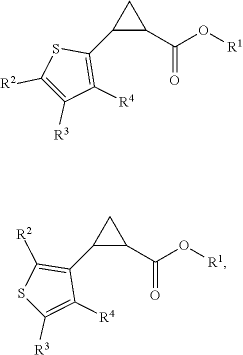

wherein R¹-R⁴ are as defined in any of the preceding formulae;
1.37 Compound of Formula I, or any of 1.1-1.31, wherein the compound of Formula I is a compound of Formula IA or IB selected from the following:

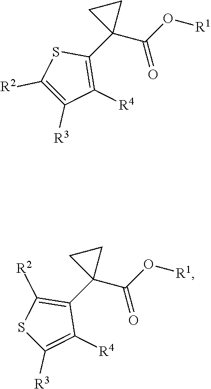

wherein R¹-R⁴ are as defined in any of the preceding formulae;
1.38 Compound of Formula I, or any of 1.1-1.31, wherein the compound of Formula I is a compound of Formula IA or IB selected from the following:

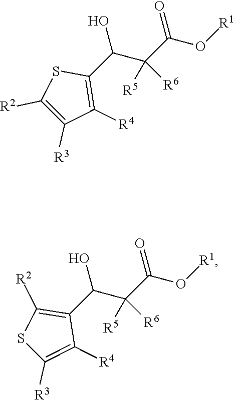

wherein R¹-R⁶ are as defined in any of the preceding formulae;
1.39 Compound of Formula I, or any of 1.1-1.31, wherein the compound of Formula I is a compound of Formula IC or ID selected from the following:

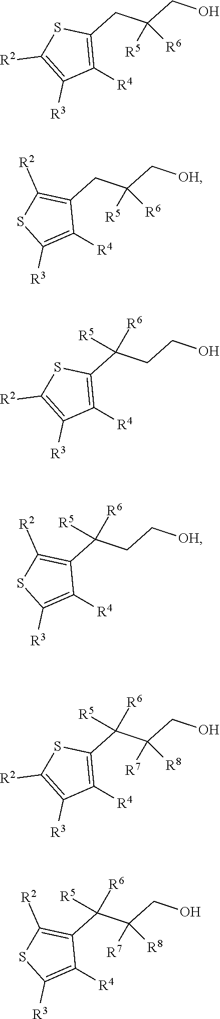

wherein R¹-R⁸ are as defined in any of the preceding formulae;
1.40 Compound of Formula I, or any of 1.1-1.39, wherein R¹ is methyl or ethyl, R²-R⁴ are each independently, H or methyl, and R⁵-R¹⁰ are each, independently, OH, H, or methyl, provided that when m or n is 0, R⁹ and R¹⁰, or R⁷ and R⁸, respectively, are absent;
1.41 Compound of Formula I or any of 1.1-1.35, wherein the compound is not a compound wherein:
   (a) m is 0, n is 0 or 1 and R¹ is C₁₋₄alkyl, and R²-R⁸ are H or C₁₋₃alkyl;
   (b) m is 0, n is 0 or 1 and R¹ is aryl (e.g., phenyl), and R²-R⁸ are H or C₁₋₃alkyl;
   (c) m is 0, n is 0 or 1 and R¹ is C₃₋₆cycloalkyl (e.g., cyclopropyl), and R²-R⁸ are H or C₁₋₃alkyl; and/or
   (d) m is 0, n is 0 and R¹ is C₂₋₄alkenyl (e.g., ethenyl), R²-R⁶ are H or C₁₋₃alkyl;
1.42 Compound of Formula I or any of 1.1-1.35, wherein the compound is not ethyl 3-(3-thienyl)propionate;
1.43 Compound of Formula I or any of 1.1-1.35, wherein:
   (a) m is 0; n is 1; R¹ is C₁₋₄alkyl (e.g., ethyl); and R²-R⁸ are H or C₁₋₃alkyl; or
   (b) m is 0; n is 1; R¹ is C₁₋₄alkyl (e.g., ethyl); and R²-R⁸ are H;
1.44 Compound of Formula I, or any of 1.1-1.35 wherein the compound of Formula I is ethyl 3-(3-thienyl)propionate;
1.45 Any preceding compound, wherein the compound has a molecular weight selected from the range of to 150 to 400, or 150 to 350, or 150 to 300, or 150 to 250, or 150 to 225, or 150 to 200, or 150 to 190;
1.46 Any preceding compound wherein the compound is selected from any one or more of the compounds of Examples 6 to 25, 42 to 76, 79 to 121, and 125-127, as defined herein;
1.47 Any preceding compound wherein the compound is selected from any one or more of the compounds of Examples 6, 7, 9, 10, 15, 50, 73, 86, 89, 90, 91, and 94, as defined herein;
1.48 Any preceding compound wherein the compound is selected from any one or more of the compounds of Examples 7 and 9 as defined herein;
1.49 Any preceding compound, wherein if the compound has an acidic or basic atom or functional group, then the compound is in the form of a salt, e.g., a base addition salt or an acid addition salt;
1.50 Any preceding compound, wherein the compound has a pleasing taste and/or aroma, e.g., as judged by a trained flavor or fragrance chemist or master perfumer (e.g., toasted coconut or other nutty aromas and/or tastes).

In another embodiment of the first aspect, the application relates to compounds of Formula IIA, IIB, IIC, IID, IIE, and IIF (collectively “Formula II”):

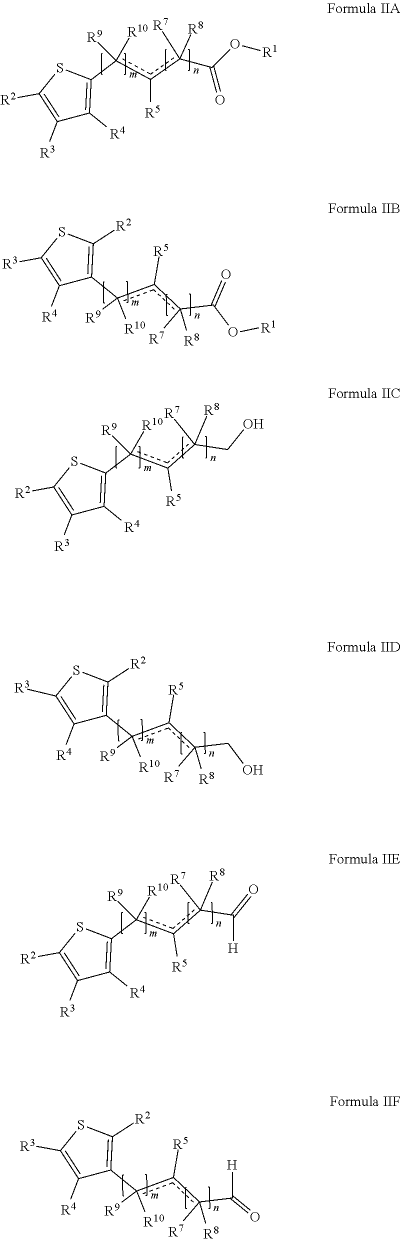

[symbol]

means either a single bond or a double bond, provided one of the [symbol] is a single bond and the other [symbol] is a double bond;
R¹ is C₁₋₄alkyl (e.g., methyl, ethyl, n-propyl, isopropyl, n-butyl, isobutyl, sec-butyl or tert-butyl), aryl (e.g., phenyl), arylC₁₋₄alkyl (e.g., benzyl), C₃₋₆cycloalkyl (e.g., cyclopropyl), C₃₋₆cycloalkyl-C₁₋₄alkyl (e.g., cyclopropylmethyl), or C₂₋₄alkenyl (e.g., propenyl or butenyl);
R², R³, and R⁴ are each independently selected from H, C₁₋₃alkyl (e.g., methyl, ethyl, n-propyl, isopropyl), C₁₋₃alkoxy (e.g., methoxy), and C₂₋₄alkenyl (e.g., propenyl);
R⁵, R⁷, and R⁸ are each independently selected from OH, H, C₁₋₃alkyl (e.g., methyl), and C₃₋₆cycloalkyl (e.g., cyclopropyl, cyclobutyl, cyclopentyl or cyclohexyl); or R⁷ and R⁸, together with the carbon atom to which they are attached, form a C₃₋₄cycloalkyl ring (e.g., a spiro-fused cyclopropyl), provided that R⁸ is absent when R⁷ is attached to a double-bonded carbon atom; or
R⁹ and R¹⁰ are both H, or R⁹ and R¹⁰ together form a C(═O) group (carbonyl group), or R⁹ is OH and R¹⁰ is H or C₁₋₃alkyl (e.g., methyl), provided that R⁹ is absent when R¹⁰ is attached to a double-bonded carbon atom; and
m and n are independently 0 or 1,
optionally, provided that when m is 0, n is 0 or 1, and R¹ is C₁₋₄alkyl, aryl (e.g., phenyl), or C₃₋₆cycloalkyl (e.g., cyclopropyl), then (i) at least one of R²-R⁸ is not H or C₁₋₃alkyl, or (ii) R²-R⁸ are not all H or C₁₋₃alkyl; and/or optionally provided that when m is 0, n is 0, and R¹ is C₂₋₄alkenyl (e.g., ethenyl), then (i) at least one of R²-R⁶ is not H or C₁₋₃alkyl, or (ii) R²-R⁶ are not all H or C₁₋₃alkyl.

In further embodiments of this aspect, the present disclosure provides:
2.1. Compound of Formula II, wherein R¹ C₁₋₄alkyl (e.g., methyl, ethyl, n-propyl, isopropyl, n-butyl, isobutyl, sec-butyl or tert-butyl), aryl (e.g., phenyl), arylC₁₋₄alkyl (e.g., benzyl), C₃₋₆cycloalkyl (e.g., cyclopropyl), C₃₋₆cycloalkyl-C₁₋₄alkyl (e.g., cyclopropylmethyl), or C₂₋₄alkenyl (e.g., propenyl or butenyl);
2.2. Compound of Formula II, wherein R¹ is C₁₋₄alkyl (e.g., methyl, ethyl, n-propyl, isopropyl, n-butyl, isobutyl, sec-butyl or tert-butyl);
2.3. Compound of Formula II, wherein R¹ is aryl (e.g., phenyl);
2.4. Compound of Formula II, wherein R¹ is arylC₁₋₄alkyl (e.g., benzyl);
2.5. Compound of Formula II, wherein R¹ is C₃₋₆cycloalkyl (e.g., cyclopropyl);
2.6. Compound of Formula II, wherein R¹ is C₃₋₆cycloalkyl-C₁₋₄alkyl (e.g., cyclopropylmethyl);
2.7. Compound of Formula II, wherein R¹ is C₂₋₄alkenyl (e.g., propenyl or butenyl);
2.8. Compound of Formula II, or any of 2.1-1.7, wherein R², R³, and R⁴ are each independently H, C₁₋₃alkyl (e.g., methyl, ethyl, n-propyl, iso-propyl), C₁₋₃alkoxy (e.g., methoxy), or C₂₋₄alkenyl (e.g., propenyl);
2.9. Compound of Formula II, or any of 2.1-1.7, wherein R², R³, and R⁴ are each H;
2.10. Compound of Formula II, or any of 2.1-1.7, wherein R², R³, and R⁴ are each independently C₁₋₃alkyl (e.g., methyl, ethyl, n-propyl, iso-propyl);
2.11. Compound of Formula II, or any of 2.1-1.7, wherein R², R³, and R⁴ are each independently C₁₋₃alkoxy (e.g., methoxy);
2.12. Compound of Formula II, or any of 2.1-1.7, wherein R², R³, and R⁴ are each independently C₂₋₄alkenyl (e.g., propenyl);
2.13. Compound of Formula II, or any of 2.1-1.12, wherein R⁵, R⁷, and R⁸ are independently OH, H, C₁₋₃alkyl (e.g., methyl), or C₃₋₆cycloalkyl (e.g., cyclopropyl, cyclobutyl, cyclopentyl or cyclohexyl), or R⁷ and R⁸, together with the carbon atom to which they are attached, form a C₃₋₄cycloalkyl ring (e.g., spiro-fused cyclopropyl);
2.14. Compound of Formula II, or any of 2.1-1.12, wherein R⁵, R⁷, and R⁸ are independently H;
2.15. Compound of Formula II, or any of 2.1-1.12, wherein R⁵, R⁷, and R⁸ are independently C₁₋₃alkyl (e.g., methyl);
2.16. Compound of Formula II, or any of 2.1-1.12, wherein R⁵, R⁷, and R⁸ are independently C₃₋₆cycloalkyl (e.g., cyclopropyl, cyclobutyl, cyclopentyl or cyclohexyl);
2.17. Compound of Formula II, or any of 2.1-1.12, wherein R⁷ and R⁸, together with the carbon atom to which they are attached, form a C₃₋₄cycloalkyl ring (e.g., spiro-fused cyclopropyl);
2.18. Compound of Formula II, or any of 2.1-1.12, wherein R⁵ is H;
2.19. Compound of Formula II, or any of 2.1-1.12, wherein R⁷ is H and R⁸ is OH;
2.20. Compound of Formula II, or any of 2.1-1.17, wherein R⁹ and R¹⁰ are H, or R⁹ and R¹⁰ together form a C(═O) group;
2.21. Compound of Formula II, or any of 2.1-1.17, wherein R⁹ and R¹⁰ are H;
2.22. Compound of Formula II, or any of 2.1-1.17, wherein R⁹ and R¹⁰ together form a C(═O) group;
2.23. Compound of Formula II, or any of 2.1-1.17, wherein R⁹ is OH and R¹⁰ is H;
2.24. Compound of Formula II, or any of 2.1-1.22, wherein m is 0 or 1;
2.25. Compound of Formula II, or any of 2.1-1.22, wherein m is 0;
2.26. Compound of Formula II, or any of 2.1-1.22, wherein m is 1;
2.27. Compound of Formula II, or any of 2.1-1.28, wherein n is 0 or 1;
2.28. Compound of Formula II, or any of 2.1-1.28, wherein n is 0;
2.29. Compound of Formula II, or any of 2.1-1.28, wherein n is 1;
2.30. Compound of Formula II, or any of 2.1-2.29, wherein the double bond has (E)-geometry;
2.31. Compound of Formula II, or any of 2.1-2.29, wherein the double bond has (Z)-geometry;
2.32. Compound of Formula II, or any of 2.1-1.31, wherein the compound of Formula II is a compound of Formula IIA or IB selected from the following:

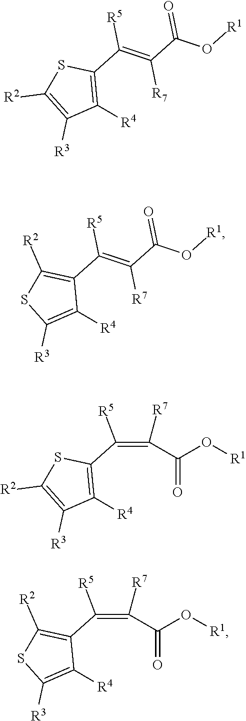

wherein R¹-R⁴ are as defined in any of the preceding formulae;
2.33. Compound of Formula II, or any of 2.1-2.32, wherein R¹ is methyl or ethyl, R²-R⁴ are each independently, H or methyl, and R⁵-R¹⁰ are each, independently, OH, H, or methyl, provided that when m or n is 0, R⁹ and R¹⁰, or R⁷ and R⁸, respectively, are absent;
2.34. Compound of Formula II or any of 2.1-1.35, wherein the compound is not a compound wherein:
   (e) m is 0, n is 0 or 1 and R¹ is C₁₋₄alkyl, and R²-R⁸ are H or C₁₋₃alkyl;
   (f) m is 0, n is 0 or 1 and R¹ is aryl (e.g., phenyl), and R²-R⁸ are H or C₁₋₃alkyl;
   (g) m is 0, n is 0 or 1 and R¹ is C₃₋₆cycloalkyl (e.g., cyclopropyl), and R²-R⁸ are H or C₁₋₃alkyl; and/or
   (h) m is 0, n is 0 and R¹ is C₂₋₄alkenyl (e.g., ethenyl), R²-R⁶ are H or C₁₋₃alkyl;
2.35. Compound of Formula II or any of 2.1-1.35, wherein:
   (c) m is 0; n is 1; R¹ is C₁₋₄alkyl (e.g., ethyl); and R²-R⁸ are H or C₁₋₃alkyl; or
   (d) m is 0; n is 1; R¹ is C₁₋₄alkyl (e.g., ethyl); and R²-R⁸ are H;
2.36. Any preceding compound, wherein the compound has a molecular weight selected from the range of to 150 to 400, or 150 to 350, or 150 to 300, or 150 to 250, or 150 to 225, or 150 to 200, or 150 to 190;
2.37. Any preceding compound, wherein the compound has (E)-stereochemistry across the double bond;
2.38. Any preceding compound, wherein the compound has (Z)-stereochemistry across the double bond;
2.39. Any preceding compound wherein the compound is selected from any one or more of the compounds of Examples 1-5, 26-41, 77, 78, and 121-124, as defined herein;
2.40. Any preceding compound wherein the compound is selected from any one or more of the compounds of Examples 31, 78, and 121, as defined herein;
2.41. Any preceding compound, wherein if the compound has an acidic or basic atom or functional group, then the compound is in the form of a salt, e.g., a base addition salt or an acid addition salt;
2.42. Any preceding compound, wherein the compound has a pleasing taste and/or aroma, e.g., as judged by a trained flavor or fragrance chemist or master perfumer (e.g., toasted coconut or other nutty aromas and/or tastes).

In a second aspect, the present disclosure provides a flavor composition and/or a fragrance composition (Composition 1) comprising a Compound of Formula I, or any of 1.1-1.50, e.g., any of compounds of Formula IA, IB, IC, ID, IE, or IF, in admixture with one or more non-toxic, orally acceptable, pharmaceutically acceptable, cosmetically acceptable, or acceptable for a household product, carriers or excipients. In particular embodiments, the second aspect provides:
1.1 Composition 1, wherein the composition is a fragrance composition.
1.2 Composition 1, wherein the composition is a flavor composition.
1.3 Composition 1, or any of Compositions 1.1-1.2, wherein the composition comprises the Compound of Formula I, or any of 1.1-1.50, in an amount of 0.1 to 100% by weight of the composition, e.g., 0.1 to 90%, or 0.1 to 80%, or 0.1 to 70%, or 0.1 to 60%, or 0.1 to 50%, or 0.1 to 40%, or 0.1 to 30%, or 0.1 to 20%, or 0.1 to 15%, or 0.1 to 10%, or 0.1 to 7.5%, or 0.1 to 5%, or 0.1 to 4%, or 0.1 to 3%, or 0.1 to 2%, or 0.1 to 1%, or 10 to 100%, or 20 to 100%, or 30 to 100%, or 40 to 100%, or 50 to 100%, or 60 to 100%, or 70 to 100%, or 80 to 100%, or 90 to 100%, or 95 to 100%, or 25 to 75%, or 50 to 75%, or 75 to 95%, by weight of the composition.
1.4 Composition 1, or any of Compositions 1.1-1.3, wherein the composition further comprises one or more other flavors or fragrances.
1.5 Composition 1, or any of Compositions 1.1-1.4, wherein the composition further comprises one or more solvents.
1.6 Composition 1.5, wherein the one or more solvents are selected from water, methanol, ethanol, propanol, isopropanol, dimethyl ether, diethyl ether, diisopropyl ether, methyl tert-butyl ether, ethylene glycol, propylene glycol, glycerin, triethyl citrate, triacetin, triglycerides, liquid waxes, propylene glycol derivatives (e.g., polypropylene glycols or ethylene oxide/propylene oxide copolymers), ethylene glycol derivatives (e.g., polyethylene glycols or ethylene oxide/propylene oxide copolymers), other alcohols or ethers, or any combination thereof.
1.7 Composition 1, or any of composition 1.1 to 1.6, wherein the composition is a liquid.
1.8 Composition 1, or any of compositions 1.1 to 1.6, wherein the composition is a soft or waxy solid.
1.9 Composition 1, or any of compositions 1.1-1.8, wherein the composition further comprises one or more of a polymer, gelling agent, powdery substrate, surfactant, emollient, plasticizer, wetting agent, swelling agent, or active agent (e.g., an oral care active or a medicinal active agent), or any other additives as described herein.
1.10 Composition 1, or any of Compositions 1.1-1.9, wherein the composition does not comprise any ingredient or component that would not be safe for ingestion, application to the oral cavity, or topical application to the skin or hair;
1.11 Composition 1, or any of Compositions 1.1-1.10, wherein the composition does not comprise any ingredient or component which is unsafe for, or not approved for, use in a food, cosmetic composition, pharmaceutical composition, oral care composition, or consumer cleaning composition.

In another embodiment of the second aspect, the present disclosure provides a flavor composition and/or a fragrance composition (Composition 2) comprising a Compound of Formula II, or any of 2.1-2.42, e.g., any of compounds of Formula IIA, IIB, IIC, IID, IIE, or IIF, in admixture with one or more non-toxic, orally acceptable, pharmaceutically acceptable, cosmetically acceptable, or acceptable for a household product, carriers or excipients. In particular embodiments, the second aspect provides:
2.1. Composition 2, wherein the composition is a fragrance composition.
2.2. Composition 2, wherein the composition is a flavor composition.
2.3. Composition 2, or any of Compositions 2.1-2.2, wherein the composition comprises the Compound of Formula I, or any of 2.1-1.50, in an amount of 0.1 to 100% by weight of the composition, e.g., 0.1 to 90%, or 0.1 to 80%, or 0.1 to 70%, or 0.1 to 60%, or 0.1 to 50%, or 0.1 to 40%, or 0.1 to 30%, or 0.1 to 20%, or 0.1 to 15%, or 0.1 to 10%, or 0.1 to 7.5%, or 0.1 to 5%, or 0.1 to 4%, or 0.1 to 3%, or 0.1 to 2%, or 0.1 to 1%, or 10 to 100%, or 20 to 100%, or 30 to 100%, or 40 to 100%, or 50 to 100%, or 60 to 100%, or 70 to 100%, or 80 to 100%, or 90 to 100%, or 95 to 100%, or 25 to 75%, or 50 to 75%, or 75 to 95%, by weight of the composition.
2.4. Composition 2, or any of Compositions 2.1-2.3, wherein the composition further comprises one or more other flavors or fragrances.
2.5. Composition 2, or any of Compositions 2.1-2.4, wherein the composition further comprises one or more solvents.
2.6. Composition 2.5, wherein the one or more solvents are selected from water, methanol, ethanol, propanol, isopropanol, dimethyl ether, diethyl ether, diisopropyl ether, methyl tert-butyl ether, ethylene glycol, propylene glycol, glycerin, triethyl citrate, triacetin, triglycerides, liquid waxes, propylene glycol derivatives (e.g., polypropylene glycols or ethylene oxide/propylene oxide copolymers), ethylene glycol derivatives (e.g., polyethylene glycols or ethylene oxide/propylene oxide copolymers), other alcohols or ethers, or any combination thereof.
2.7. Composition 2, or any of composition 2.1 to 2.6, wherein the composition is a liquid.
2.8. Composition 2, or any of compositions 2.1 to 2.6, wherein the composition is a soft or waxy solid.
2.9. Composition 2, or any of compositions 2.1-2.8, wherein the composition further comprises one or more of a polymer, gelling agent, powdery substrate, surfactant, emollient, plasticizer, wetting agent, swelling agent, or active agent (e.g., an oral care active or a medicinal active agent), or any other additives as described herein.
2.10. Composition 2, or any of Compositions 2.1-2.9, wherein the composition does not comprise any ingredient or component that would not be safe for ingestion, application to the oral cavity, or topical application to the skin or hair;
2.11. Composition 2, or any of Compositions 2.1-2.10, wherein the composition does not comprise any ingredient or component which is unsafe for, or not approved for, use in a food, cosmetic composition, pharmaceutical composition, oral care composition, or consumer cleaning composition.

As used herein, the term “fragrance composition” means a mixture of fragrance ingredients (e.g., including a Compound of Formula I or any of 1.1-1.50, or Compound of Formula II, or any of 2.1-2.42) with one or more non-toxic, cosmetically acceptable, or acceptable for a household product, carriers or excipients, such as solvents. For example, the fragrance ingredient(s) may be dissolved in a suitable solvent or mixed with a powdery substrate, with additional auxiliary substances added (e.g., additives), if desired. A fragrance composition is used, and intended to be used, to provide or impart a desired odor or aroma to a product, such as a cosmetic product or household product (e.g., household cleaners). Thus, a fragrance composition is used as an ingredient or component in a final product, such as a cosmetic product or consumer product, for which a particular fragrance is desired. Examples of products having fragrance compositions include, but are not limited to, perfumes, soaps, insect repellants and insecticides, detergents, household cleaning agents, air fresheners, room sprays, pomanders, candles, cosmetics, toilet waters, pre- and aftershave lotions, talcum powders, hair-care products, body deodorants, anti-perspirants, and pet litter. A fragrance composition should have enough of its fragrance ingredients so that it is effective to provide the desired odor or aroma to the final product, and this depends both on the concentration of the fragrance ingredients in the composition and the concentration of the composition used in the product.

As used herein, the term “flavor composition” means a mixture of flavor ingredients (e.g., including a Compound of Formula I or a Compound of Formula II) with one or more non-toxic, orally acceptable, or pharmaceutically acceptable, carriers or excipients, such as solvents. For example, the flavor ingredient(s) may be dissolved in a suitable solvent or mixed with a suitable solid, semi-solid, or liquid excipients, with additional auxiliary substances added (e.g., additives), if desired. A flavor composition is used, and intended to be used, to provide or impart a desired flavor and aroma to a product, such as a food product or oral pharmaceutical product. Thus, a flavor composition is used as an ingredient or component in a final product, such as a food or oral pharmaceutical product, for which a particular flavor is desired. Examples of products having flavor compositions include, but are not limited to, oral care compositions (e.g., dental hygiene products such as mouth wash, toothpaste, floss, and breath fresheners), pharmaceutical compositions (e.g., orally administered medications including liquids, tablets or capsules), and food products. A flavor composition should have enough of its flavor ingredients so that it is effective to provide the desired flavor and aroma to the final product, and this depends both on the concentration of the flavor ingredients in the composition and the concentration of the composition used in the product.

Fragrance and flavor ingredients and mixtures of fragrance and flavor ingredients that may be used in combination with the disclosed compound for the manufacture of fragrance and flavor compositions include, but are not limited to, natural products including extracts, animal products and essential oils, absolutes, resinoids, resins, and concretes, and synthetic fragrance materials which include, but are not limited to, alcohols, aldehydes, ketones, ethers, acids, esters, acetals, phenols, ethers, lactones, furansketals, nitriles, acids, and hydrocarbons, including both saturated and unsaturated compounds and aliphatic carbocyclic and heterocyclic compounds, and animal products. As used herein, the terms “fragrance ingredient” and “flavor ingredient” refer to ingredients other than the Compounds of Formula I or Formula II which are used to impart a flavor or a fragrance to a composition or product.

Examples of esters which may be used as fragrance ingredients or flavor ingredients in the compositions and products of the present disclosure include, but are not limited to, acrylic acid esters (methyl, ethyl, etc.), acetoacetic acid esters (methyl, ethyl, etc.), anisic acid esters (methyl, ethyl, etc.), benzoic acid esters (allyl, isoamyl, ethyl, geranyl, linalyl, phenylethyl, hexyl, cis-3-hexenyl, benzyl, methyl, etc.), anthranilic acid esters (cinnamyl, cis-3-hexenyl, methyl, ethyl, linalyl, isobutyl, etc.), N-methylanthranilic acid esters (methyl, ethyl, etc.), isovaleric acid esters (amyl, allyl, isoamyl, isobutyl, isopropyl, ethyl, octyl, geranyl, cyclohexyl, citronellyl, terpenyl, linalyl, cinnamyl, phenylethyl, butyl, propyl, hexyl, benzyl, methyl, rhodinyl, etc.), isobutyric acid esters (isoamyl, geranyl, citronellyl, terpenyl, cinnamyl, octyl, nellyl, phenylethyl, phenylpropyl, phenoxyethyl, butyl, propyl, isopropyl, hexyl, benzyl, methyl, ethyl, linalyl, rhodinyl, etc.), undecylenic acid esters (allyl, isoamyl, butyl, ethyl, methyl, etc.), octanoic acid esters (allyl, isoamyl, ethyl, octyl, hexyl, butyl, methyl, linalyl, etc.), octenoic acid esters (methyl, ethyl, etc.), octynecarboxylic acid esters (methyl, ethyl, etc.), caproic acid esters (allyl, amyl, isoamyl, methyl, ethyl, isobutyl, propyl, hexyl, cis-3-hexenyl, trans-2-hexenyl, linalyl, geranyl, cyclohexyl, etc.), hexenoic acid esters (methyl, ethyl, etc.), valeric acid esters (amyl, isopropyl, isobutyl, ethyl, cis-3-hexenyl, trans-2-hexenyl, cinnamyl, phenylethyl, methyl, etc.), formic acid esters (anisyl, isoamyl, isopropyl, ethyl, octyl, geranyl, citronellyl, cinnamyl, cyclohexyl, terpenyl, phenylethyl, butyl, propyl, hexyl, cis-3-hexenyl, benzyl, linalyl, rhodinyl, etc.), crotonic acid esters (isobutyl, ethyl, cyclohexyl, etc.), cinnamic acid esters (allyl, ethyl, methyl, isopropyl, propyl, 3-phenylpropyl, benzyl, cyclohexyl, methyl, etc.), succinic acid esters (monomenthyl, diethyl, dimethyl, etc.), acetic acid esters (anisyl, amyl, α-amylcinnamyl, isoamyl, isobutyl, isopropyl, isobornyl, isoeugenyl, eugenyl, 2-ethylbutyl, ethyl, 3-octyl, p-cresyl, o-cresyl, geranyl, α- or β-santalyl, cyclohexyl, cycloneryl, dihydrocuminyl, dimethyl benzyl carbinyl, cinnamyl, styralyl, decyl, dodecyl, terpenyl, guainyl, neryl, nonyl, phenyl ethyl, phenylpropyl, butyl, furfuryl, propyl, hexyl, cis-3-hexenyl, trans-2-hexenyl, cis-3-nonenyl, cis-6-noneyl, cis-3-cis-6-nonadienyl, 3-methyl-2-butenyl, heptyl, benzyl, bornyl, myrcenyl, dihydromyrcenyl, myrtenyl, methyl, 2-methylbutyl, menthyl, linalyl, rhodinyl, etc.), salicylic acid esters (allyl, isoamyl, phenyl, phenylethyl, benzyl, ethyl, methyl, etc.), cyclohexylalkanoic acid esters (ethyl cyclohexylacetate, allyl cyclohexylpropionate, allyl cyclohexylbutyrate, allyl cyclohexylhexanoate, allyl cyclohexyldecanoate, allyl cyclohexylvalerate, etc.), stearic acid esters (ethyl, propyl, butyl, etc.), sebacic acid esters (diethyl, dimethyl, etc.), decanoic acid esters (isoamyl, ethyl, butyl, methyl, etc.), dodecanoic acid esters (isoamyl, ethyl, butyl, etc.), lactic acid esters (isoamyl, ethyl, butyl, etc.), nonanoic acid esters (ethyl, phenylethyl, methyl, etc.), nonenoic acid esters (allyl, ethyl, methyl, etc.), hydroxyhexanoic acid esters (ethyl, methyl, etc.), phenylacetic acid esters (isoamyl, isobutyl, ethyl, geranyl, citronellyl, cis-3-hexenyl, methyl, etc.), phenoxyacetic acid esters (allyl, ethyl, methyl, etc.), furancarboxylic acid esters (ethyl furancarboxylate, methyl furancarboxylate, hexyl furancarboxylate, isobutyl furaneopentyl glycol diacetateropionate, etc.), propionic acid esters (anisyl, allyl, ethyl, amyl, isoamyl, propyl, butyl, isobutyl, isopropyl, benzyl, geranyl, cyclohexyl, citronellyl, cinnamyl, tetrahydrofurfuryl, tricyclodecenyl, heptyl, bornyl, methyl, menthyl, linallyl, terpenyl, α-methylpropionyl, β-methylpropionyl, etc.), heptanoic acid esters (allyl, ethyl, octyl, propyl, methyl, etc.), heptinecarboxylic acid esters (allyl, ethyl, propyl, methyl, etc.), myristic acid esters (isopropyl, ethyl, methyl, etc.), phenylglycidic acid esters (ethyl phenylglycidate, ethyl 3-methylphenylglycidate, ethyl p-methyl-β-phenylglycidate, etc.), 2-methylbutyric acid esters (methyl, ethyl, octyl, phenyl ethyl, butyl, hexyl, benzyl, etc.), 3-methylbutyric acid esters (methyl, ethyl, etc.), butyric acid esters (anisyl, amyl, allyl, isoamyl, methyl, ethyl, propyl, octyl, guainyl, linallyl, geranyl, cyclohexyl, citronellyl, cinnamyl, nellyl, terpenyl, phenylpropyl, β-phenylethyl, butyl, hexyl, cis-3-hexenyl, trans-2-hexenyl, benzyl, rhodinyl, etc.), and hydroxybutyric acid esters (methyl, ethyl, menthyl or the like of 3-hydroxybutyric acid esters).

Examples of alcohols which may be used as fragrance ingredients or flavor ingredients, or as solvents, in the compositions and products of the present disclosure include, but are not limited to, aliphatic alcohols (isoamyl alcohol, 2-ethylhexanol, 1-octanol, 3-octanol, 1-octene-3-ol, 1-decanol, 1-dodecanol, 2,6-nonadienol, nonanol, 2-nonanol, cis-6-nonenol, trans-2, cis-6-nonadienol, cis-3, cis-6-nonadienol, butanol, hexanol, cis-3-hexenol, trans-2-hexenol, 1-undecanol, heptanol, 2-heptanol, 3-methyl-1-pentanol, etc.); terpene alcohols (borneol, isoborneol, carveol, geraniol, α- or β-santalol, citronellol, 4-thujanol, terpineol, 4-terpineol, nerol, myrcenol, myrtenol, dihydromyrcenol, tetrahydromyrcenol, nerolidol, hydroxycitronellol, farnesol, perilla alcohol, rhodinol, linalool, etc.); and aromatic alcohols (anisic alcohol, α-amylcinnamic alcohol, isopropylbenzylcarbinol, carvacrol, cumin alcohol, dimethylbenzylcarbinol, cinnamic alcohol, phenyl allyl alcohol, phenylethylcarbinol, β-phenylethyl alcohol, 3-phenylpropyl alcohol, benzyl alcohol, etc.).

Examples of aldehydes which may be used as fragrance ingredients or flavor ingredients in the compositions and products of the present disclosure include, but are not limited to, aliphatic aldehydes (acetaldehyde, octanal, nonanal, decanal, undecanal, 2,6-dimethyl-5-heptanal, 3,5,5-trimethylhexanal, cis-3, cis-6-nonadienal, trans-2, cis-6-nonadienal, valeraldehyde, propanal, isopropanal, hexanal, trans-2-hexenal, cis-3-hexenal, 2-pentenal, dodecanal, tetradecanal, trans-4-decenal, trans-2-tridecenal, trans-2-dodecenal, trans-2-undecenal, 2,4-hexadienal, cis-6-nonenal, trans-2-nonenal, 2-methylbutanal, etc.); aromatic aldehydes (anisic aldehyde, α-amylcinnamic aldehyde, α-methylcinnamic aldehyde, cyclamen aldehyde, p-isopropylphenylacetaldehyde, ethylvanillin, cumin aldehyde, salicylaldehyde, cinnamic aldehyde, o-, m- or p-tolylaldehyde, vanillin, piperonal, phenylacetaldehyde, heliotropin, benzaldehyde, 4-methyl-2-pheny-2-pentenal, p-methoxycinnamic aldehyde, p-methoxybenzaldehyde, etc.); and terpene aldehydes (geranial, citral, citronellal, α-sinensal, β-sinensal, perillaldehyde, hydroxycitronellal, tetrahydrocitral, myrtenal, cyclocitral, isocyclocitral, citronellyloxyacetaldehyde, neral, α-methylenecitronellal, myracaldehyde, vernaldehyde, safranal, etc.).

Examples of ketones which may be used as fragrance ingredients or flavor ingredients in the compositions and products of the present disclosure include, but are not limited to, cyclic ketones (1-acetyl-3,3-dimethyl-1-cyclohexene, cis-jasmone, α-, β- or γ-irone, ethyl maltol, cyclotene, dihydronootkatone, 3,4-dimethyl-1,2-cyclopentadione, sotolon, α-, β-, γ- or δ-damascone, α-, β- or γ-damascenone, nootkatone, 2-sec-butylcyclohexanone, maltol, α-, β- or γ-ionone, α-, β- or γ-methylionone, α-, β- or γ-isomethylionone, furaneol, camphor, etc.); aromatic ketones (acetonaphthone, acetophenone, anisylideneacetone, raspberry ketone, p-methyl acetophenone, anisylacetone, p-methoxy acetophenone, etc.); and chain ketones (diacetyl, 2-nonanone, diacetyl, 2-heptanone, 2,3-heptanedione, 2-pentanone, methyl amyl ketone, methyl nonyl ketone, β-methyl naphthyl ketone, methyl heptanone, 3-heptanone, 4-heptanone, 3-octanone, 2,3-hexanedione, 2-undecanone, dimethyloctenone, 6-methyl-5-hepten-2-one, etc.).

Examples of acetals which may be used as fragrance ingredients or flavor ingredients in the compositions and products of the present disclosure include, but are not limited to, acetaldehyde diethyl acetal, acetaldehyde diamyl acetal, acetaldehyde dihexyl acetal, acetaldehyde propylene glycol acetal, acetaldehyde ethyl cis-3-hexenyl acetal, benzaldehyde glycerin acetal, benzaldehyde propylene glycol acetal, citral dimethyl acetal, citral diethyl acetal, citral propylene glycol acetal, citral ethylene glycol acetal, phenylacetaldehyde dimethyl acetal, citronellyl methyl acetal, acetaldehyde phenylethylpropyl acetal, hexanal dimethyl acetal, hexanal dihexyl acetal, hexanal propylene glycol acetal, trans-2-hexenal diethyl acetal, trans-2-hexenal propylene glycol acetal, cis-3-hexenal diethyl acetal, heptanal diethyl acetal, heptanal ethylene glycol acetal, octanal dimethyl acetal, nonanal dimethyl acetal, decanal dimethyl acetal, decanal diethyl acetal, 2-methylundecanal dimethyl acetal, citronellal dimethyl acetal, Ambersage (manufactured by Givaudan), ethyl acetoacetate ethylene glycol acetal, and 2-phenylpropanal dimethyl acetal.

Examples of phenols which may be used as fragrance ingredients or flavor ingredients in the compositions and products of the present disclosure include, but are not limited to, eugenol, isoeugenol, 2-methoxy-4-vinylphenol, thymol, carvacrol, guaiacol, and chavicol, and vanillin.

Examples of ethers and epoxides which may be used as fragrance ingredients or flavor ingredients in the compositions and products of the present disclosure, but are not limited to, anethole, 1,4-cineole, dibenzyl ether, linalool oxide, limonene oxide, nerol oxide, rose oxide, methyl isoeugenol, methyl chavicol, isoamyl phenyl ethyl ether, β-napthyl methyl ether, phenyl propyl ether, p-cresyl methyl ether, vanillyl butyl ether, α-terpinyl methyl ether, citronellyl ethyl ether, geranyl ethyl ether, rose furan, theaspirane, decylmethyl ether, and methylphenyl methyl ether.

Examples of lactones which may be used as fragrance ingredients or flavor ingredients in the compositions and products of the present disclosure include, but are not limited to, γ- or δ-decalactone, γ-heptalactone, γ-nonalactone, γ- or 6-hexylactone, γ- or δ-octalactone, γ- or δ-undecalactone, δ-dodecalactone, δ-2-decenolactone, methyl lactone, 5-hydroxy-8-undecenoic acid δ-lactone, jasmine lactone, menthalactone, dihydrocoumarin, octahydrocoumarin, and 6-methylcoumarin.

Examples of furans which may be used as fragrance ingredients or flavor ingredients in the compositions and products of the present disclosure include, but are not limited to, furan, 2-methylfuran, 3-methylfuran, 2-ethylfuran, 2,5-diethyltetrahydrofuran, 3-hydroxy-2-methyltetrahydrofuran, 2-(methoxymethyl)furan, 2,3-dihydrofuran, furfural, 5-methylfurfural, 3-(2-furyl)-2-methyl-2-propenal, 5-(hydroxymethyl)furfural, 2,5-dimethyl-4-hydroxy-3(2H)-furanone (furaneol), 4,5-dim ethyl-3-hydroxy-2(5H)-furanone (sotolon), 2-ethyl-4-hydroxy-5-methyl-3(2H)-furanone (homofuraneol), 5-ethyl-3-hydroxy-4-methyl-2(5H)-furanone (homosotolon), 3-methyl-1,2-cyclopentanedione (cyclotene), 2(5H)-furanone, 4-methyl-2(5H)-furanone, 5-methyl-2(5H)-furanone, 2-methyl-3(2H)-furanone, 5-methyl-3(2H)-furanone, 2-acetylfuranone, 2-acetyl-5-methylfuran, furfuryl alcohol, methyl 2-furancarboxylate, ethyl 2-furancarboxylate, and furfuryl acetate.

Examples of hydrocarbons which may be used which may be used as fragrance ingredients or flavor ingredients in the compositions and products of the present disclosure include, but are not limited to, α- or β-bisabolene, β-caryophyllene, p-cymene, terpinene, terpinolene, cadinene, cedrene, longifolene, farnesene, limonene, ocimene, myrcene, α- or β-pinene, 1,3,5-undecatriene and valencene.

Examples of acids that may be used which may be used as fragrance ingredients or flavor ingredients in the compositions and products of the present disclosure include, but are not limited to, geranic acid, dodecanoic acid, myristic acid, stearic acid, lactic acid, phenylacetic acid, pyruvic acid, trans-2-methyl-2-pentenoic acid, 2-methyl-cis-3-pentenoic acid, 2-methyl-4-pentenoic acid, and cyclohexanecarboxylic acid.

The fragrance and flavor compositions of the application may comprise as additional fragrance or flavor ingredients one or more natural extracts or oils including, but not limited to, anise, orange, lemon, lime, mandarin, petitgrain, bergamot, lemon balm, grapefruit, elemi, olibanum, lemongrass, neroli, marjoram, angelica root, star anise, basil, bay, calamus, chamomile, caraway, cardamom, cassia, cinnamon, pepper, perilla, cypress, oregano, cascarilla, ginger, parsley, pine needle, sage, hyssop, tea tree, mustard, horseradish, clary sage, clove, cognac, coriander, estragon, eucalyptus, fennel, guaiac wood, dill, cajuput, wormseed, pimento, juniper, fenugreek, garlic, laurel, mace, myrrh, nutmeg, spruce, geranium, citronella, lavender, lavandin, palmarosa, rose, rosemary, sandalwood, oakmoss, cedarwood, vetiver, linaloe, bois de rose, patchouli, labdanum, cumin, thyme, ylang lignaloe, birch, capsicum, celery, tolu balsam, genet, immortelle, benzoin, jasmine, cassie, tuberose, reseda, marigold, mimosa, opoponax, orris, vanilla and licorice. Each of these natural extracts or oils comprises a complex mixture of chemical compounds, which may include those compounds described above. Additional fragrance ingredients may be isolated from natural products, for example, geraniol and citronellal may be isolated from citronella oil, citral may be isolated from lemon-grass oil, eugenol may be isolated from clove oil, and linalool may be isolated from rosewood oil. Animal products used in fragrance compositions include, but are not limited to, musk, ambergris, civet and castoreum. The natural ingredients described herein may also be produced synthetically, and may include the compounds disclosed herein, and be used as fragrance and/or flavor ingredients in the fragrance and flavor compositions of the present application.

Examples of fragrance ingredients used in perfumes, air fresheners, laundry detergents, pet litters, cleaning products, liquid and bar soaps, shampoos and conditioners, cosmetics, deodorants, and personal hygiene products include, but are not limited to: hexyl cinnamic aldehyde; amyl cinnamic aldehyde; amyl salicylate; hexyl salicylate; terpineol; 3,7-dimethyl-cis-2,6-octadien-1-ol; 2,6-dimethyl-2-octanol; 2,6-dimethyl-7-octen-2-ol; 3,7-dimethyl-3-octanol; 3,7-dimethyl-trans-2,6-octadien-1-ol; 3,7-dimethyl-6-octen-1-ol; 3,7-dimethyl-1-octanol; 2-methyl-3-(para-tert-butylphenyl)-propionaldehyde; 4-(4-hydroxy-4-methylpentyl)-3-cyclohexene-1-carboxaldehyde; tricyclodecenyl propionate; tricyclodecenyl acetate; anisaldehyde; 2-methyl-2-(para-iso-propylphenyl)-propionaldehyde; ethyl-3-methyl-3-phenyl glycidate; 4-(para-hydroxyphenyl)-butan-2-one; 1-(2,6,6-trimethyl-2-cyclohexen-1-yl)-2-buten-1-one; para-methoxyacetophenone; para-methoxy-alpha-phenylpropene; methyl-2-n-hexyl-3-oxo-cyclopentane carboxylate; undecalactone gamma, geraniol; geranyl acetate; linalool; linalyl acetate; tetrahydrolinalool; citronellol; citronellyl acetate; dihydromyrcenol; dihydromyrcenyl acetate; tetrahydromyrcenol; terpinyl acetate; nopol; nopyl acetate; 2-phenylethanol; 2-phenylethyl acetate; benzyl alcohol; benzyl acetate; benzyl salicylate; benzyl benzoate; styrallyl acetate; dimethylbenzylcarbinol; trichloromethylphenylcarbinyl methylphenylcarbinyl acetate; isononyl acetate; vetiveryl acetate; vetiverol; 2-methyl-3-(p-tert-butylphenyl)-propanal; 2-methyl-3-(p-isopropylphenyl)-propanal; 3-(p-tert-butylphenyl)-propanal; 4-(4-methyl-3-pentenyl)-3-cyclohexenecarbaldehyde; 4-acetoxy-3-pentyltetrahydropyran; methyl dihydrojasmonate; 2-n-heptylcyclopentanone; 3-methyl-2-pentyl-cyclopentanone; n-decanal; n-dodecanal; 9-decenol-1; phenoxyethyl isobutyrate; phenylacetaldehyde dimethylacetal; phenylacetaldehyde diethylacetal; geranonitrile; citronellonitrile; cedryl acetal; 3-isocamphylcyclohexanol; cedryl methyl ether; isolongifolanone; aubepine nitrile; aubepine; heliotropine; eugenol; vanillin; diphenyl oxide; hydroxycitronellal ionones; methyl ionones; isomethyl ionones; irones; cis-3-hexenol and esters thereof; indane musk fragrances; tetralin musk fragrances; isochroman musk fragrances; macrocyclic ketones; macrolactone musk fragrances; and ethylene brassylate.

The fragrance and flavor ingredients in a given product's fragrance or flavor composition are selected based on the intended use of the product and the product's desired aroma. For example, flavor ingredients used in toothpaste, mouth wash, and dental hygiene products may be selected to impart “freshness” and include, but are not limited to, spearmint oil, peppermint oil, star anise oil, lemon oil, and menthol.

Flavor compositions may be used to mask the unpleasant taste of orally administered medications. For example, if a medication is salty, a flavor composition that has cinnamon, raspberry, orange, maple, butterscotch, or glycyrrhiza (licorice) flavor may be used to mask the taste. If a medication is overly sweet, a flavor composition that has a berry, vanilla, or acacia flavor may render the medication more palatable. In the case of bitter tasting medications, flavor compositions that have cocoa, chocolate-mint, wild cherry, walnut, glycyrrhiza (licorice), and eriodictyon flavors might be used, whereas sour medications may be improved by flavor compositions that have fruity, citrus, or cherry flavors. These flavors may be provided by the natural or synthetic flavor ingredients discussed herein.

Examples of flavor ingredients used in flavor compositions for food products also include, but are not limited to, glucosyl steviol glycosides, isomenthols, carbonothoic acids, cassyrane, 1,5-octadien-3-ol, 2-mercaptoheptan-4-ol, 4 3-(methylthio)decanal, (4Z,7Z)-trideca-4,7-dienal, persicaria odorata oil, Amacha leaves extract, glutamyl-2-aminobutyric acid, glutamyl-2-aminobutyric acid, glutamyl-norvalyl-glycine, glutamyl-norvaline, N1-(2,3-Dimethoxybenzyl)-N2-(2-(pyridin-2-yl)ethyl) oxalamide, 1-(2-hydroxy-4-methylcyclohexyl)ethanone, Mexican lime oil, Persian lime oil, 6-methoxy-2,6-dimethylheptanal, 3,5-undecadien-2-one, 2,5-undecadien-1-ol, triethylthialdine. 4-methylpentyl 4-methylvalerate, (R)−N-(1-methoxy-4-methylpentan-2-yl)-3,4-dimethylbenzamide, 2 N-acetyl glutamate, 1,3-propanediol, Szechuan pepper extract, Tasmannia lanceolata extract, Mentha longifolia oil, mangosteen distillate, ethyl 3-(2-hydroxyphenyl)propanoate, 1-cyclopropanemethyl-4-methoxybenzene, prenyl thioisobutyrate, prenyl thioisovalerate, matairesinol, stevioside, 1-(2,4-dihydroxyphenyl)-3-(3-hydroxy-4-methoxyphenyl)propan-1-one, ethyl 5-formyloxydecanoate, 3-[3-(2-isopropyl-5-methyl-cyclohexyl)ureido]butyric acid ethyl ester, 2-Isopropyl-4-methyl-3-thiazoline, 2,6,10-trimethyl-9-undecenal, 5-mercapto-5-methyl-3-hexanone, Meyer lemon oil, teviol glycoside extract, Stevia rebaudiana, rebaudioside A 60%, rubescenamine, 4-amino-5-(3-(isopropylamino)-2,2-dimethyl-3-oxopropoxy)-2-methylquinoline-3-carboxylic acid, 3-methyl-5-(2,2,3-trimethylcyclopent-3-en-1-yl)pent-4-en-2-ol, (1-Methyl-2-(1,2,2-trimethylbicyclo[3.1.0]hex-3-ylmethyl)cyclopropyl)methanol, erospicata oil, and curly mint oil. See L. J. Marnett et al., GRAS Flavoring Substances 26, Food Technology, 44-45 (2013).

Preferred solvents and excipients for use in the compositions and products of the present disclosure include, but are not limited to, triethyl citrate, triacetin, glycerol, propylene glycol, dipropylene glycol, isopropyl myristate, ethanol, water, triglycerides, liquid waxes, propylene glycol derivatives (e.g., polymers), and ethylene glycol derivatives (e.g., polymers).

The amount of a given fragrance or flavor ingredient in a fragrance or flavor composition cannot be categorically described because it varies depending on the type product being scented or flavored, the intended use of the product, and the desired aroma and/or taste of the product. The amount of a fragrance or flavor ingredient in a fragrance or flavor composition is usually in the range of from about 1% to about 99% by mass of the fragrance composition. When the amount of the ingredient is too small, a sufficient strength of the scent or flavor may not be obtained. Further, when the amount of the ingredient is too large, a larger amount of the agent(s) needed to solubilize the ingredient may be needed, which may in turn reduce the desired aromatic or flavor properties of the end product by inhibiting volatilization or other mechanisms by which the flavor or fragrance is dispersed when the product is used or consumed. The amount of each of the fragrance and flavor ingredients in a given fragrance or flavor composition must therefore be selected based upon the aromatic and/or flavor characteristics of the selected ingredient, the overall composition of the product, and the intended aromatic and/or flavor effect.

Additives may be used in the flavor and fragrance compositions of the present disclosure. Additives that may be used include, but are not limited to, solvents, surfactants, pH adjusters, buffers, thickening agents, desiccants, emulsifiers, foaming agents, stabilizers, antioxidants, and disintegrating agents. Other fragrance and flavor composition additives will be selected in accordance with the intended use of the composition.

Solvents, for example water-soluble organic solvents, which may be used in the flavor and fragrance compositions of the present disclosure include, but are not limited to, ethanol, propanol, isopropanol, butanol, 3-methoxy-3-methyl-1-butanol, benzyl alcohol, ethyl carbitol (diethylene glycol monoethyl ether), ethylene glycol, propylene glycol, dipropylene glycol, butylene glycol, hexylene glycol, glycerin, ethylene glycol monomethyl ether, propylene glycol monomethyl ether, propylene glycol monoethyl ether, and dipropylene glycol monomethyl ether. These water-soluble solvents may be used solely or in combination. The content of the water-soluble organic solvent in the compositions of the application may be determined according to the desired composition properties, and is usually from about 1% to about 99% by mass.

Oil-soluble organic solvents which may be used with the flavor and fragrance compositions of the application include, but are not limited to, isoparaffin, paraffin, limonene, pinene, triethyl citrate, benzyl benzoate, isopropyl myristate, triacetin, and silicone.

Preferred solvents include, but are not limited to, triethyl citrate, triacetin, glycerol, ethanol, water, triglycerides, liquid waxes, propylene glycol derivatives, and ethylene glycol derivatives.

In some embodiments, the flavor and fragrance compositions and products of the present disclosure may further comprise other substances, including, but not limited to, sequestering agents, preservatives, antioxidants, deodorizers, sterilization agents, ultraviolet absorbers, pH adjusters, insecticidal components, components for protection from insects, insect repellents, colorants, excipients, and buffers. The substances used in, or in addition to, the fragrance and flavor compositions of the present application may be determined by the product in which the composition is included. When the substance is used in a flavor or fragrance composition, it may be an additive. When the substance is used alongside a flavor or fragrance composition, it may be considered as part of a product composition that comprises a fragrance or flavor composition.

Excipients that may be used in the fragrance and flavoring compositions and products of the present disclosure may vary depending on the use of the intended product and its overall composition. In some instances, the excipient may be included in the fragrance or flavor composition or may, alternatively, be independent of the composition. Excipients used in or with flavoring compositions of an orally administered medication may include, but are not limited to, tablet coatings, such as a cellulose ether hydroxypropyl methylcellulose, synthetic polymer, shellac, corn protein zein or other polysaccharides, and gelatin. In contrast, cosmetic excipients may include, but are not limited to, Carbopol 940 ETD, triethanolamine, purified water, glycerin, imidazolidinyl urea, EDTA, polyvinyl alcohol, methyl parabens phenoxyethanol 0, ethyl alcohol 1, peg 7 glyceryl cocoate, peg 6 triglyceryl caproic glycerides, acemulogar LAM V, isopropyl myristate, tegosoft CT, xanthan gum, sepicide CL, polyquaternium 7, and Vaseline oils. Additional suitable excipients for use with or in a flavor and/or fragrance composition for a given product will be readily selected by those having ordinary skill in the art.

Buffers that may be used with the fragrance and flavoring compositions of the present application may vary depending on the use of the intended product and its overall composition. In some instances, the buffer may be included in the fragrance or flavor composition or may, alternatively, be independent of the composition. Examples of buffers that may be used in or with the fragrance and flavor compositions of the application include, but are not limited to, citrates, acetates, and phosphates. For example, trisodium citrate may be used as a flavor or as a preservative, and is known to impart tartness to a flavor, but also acts as a buffer. Trisodium citrate is an ingredient in a variety of sodas and other beverages, as well as drink mixes and bratwurst. In cosmetic products, disodium hydrogen phosphate, potassium dihydrogen phosphate, disodium hydrogen phosphate and, and citric acid may be used to buffer the pH of the product. In toothpaste, calcium carbonate and/or dicalcium phosphate may be used as pH buffers. Additional suitable buffers for use with or in a flavor and/or fragrance composition for a given product will be readily selected by those having ordinary skill in the art.

In a fourth aspect, the present disclosure provides a product which comprises Composition 1 or any of 1.1 to 1.11. In some embodiments, the product may be selected from the following: personal care products (e.g., a soap, skin cream or lotion, balm, shampoo, body wash, shower gel, hydrating cream, deodorant, antiperspirant, after-shave lotion, cologne, perfume, or other hair care or skin care product), sunscreens, insect repellants and insecticides, detergents, household cleaning agents (e.g., a surface cleaner, a metal cleaner, a wood cleaner, a glass cleaner, a body cleaner such as a soap, a dish-washing detergent, or a laundry detergent), air fresheners, room sprays, pomanders, candles, cosmetics (e.g., perfumes, colognes, nail polish, eye liner, mascara, lipstick, foundation, concealer, blush, bronzer, eye shadow, lip liner, lip balm), toilet waters, talcum powders, and pet litter.

In another embodiment of the fourth aspect, the present disclosure provides a product which comprises Composition 2 or any of 2.1 to 2.11. In some embodiments, the product may be selected from the following: personal care products (e.g., a soap, skin cream or lotion, balm, shampoo, body wash, shower gel, hydrating cream, deodorant, antiperspirant, after-shave lotion, cologne, perfume, or other hair care or skin care product), sunscreens, insect repellants and insecticides, detergents, household cleaning agents (e.g., a surface cleaner, a metal cleaner, a wood cleaner, a glass cleaner, a body cleaner such as a soap, a dish-washing detergent, or a laundry detergent), air fresheners, room sprays, pomanders, candles, cosmetics (e.g., perfumes, colognes, nail polish, eye liner, mascara, lipstick, foundation, concealer, blush, bronzer, eye shadow, lip liner, lip balm), toilet waters, talcum powders, and pet litter.

Having now described some embodiments of the application, it should be apparent to those skilled in the art that the foregoing is merely illustrative and not limiting, having been presented by way of example only. The embodiments of the application can therefore be in other specific forms without departing from the spirit or essential characteristics thereof.

Those skilled in the art should recognize or be able to ascertain, using no more than routine experimentation, equivalents to the specific embodiments of the application. It is therefore to be understood that the embodiments described herein are presented by way of example only and that the scope of the application is thus indicated by the appended claims and equivalents thereto, and that the application may be practiced otherwise than as specifically described in the foregoing description.

The term “about,” when used to describe one of the compositions of the application, refers to a recited percentage ±5%, ±4%, ±3%, ±2.5%, ±2%, ±1.5%, ±1%, ±0.75%, ±0.5%, ±0.25%, or ±0.1%. In one embodiment, the term “about,” refers to a recited percentage ±5%. For example, “about 50%” refers to the range 45% to 55%. In one embodiment, the term “about,” refers to a recited percentage ±2.5%. In one embodiment, the term “about,” refers to a recited percentage ±1%. In one embodiment, the term “about,” refers to a recited percentage ±0.5%. In one embodiment, the term “about,” refers to a recited percentage ±0.1%.

As used herein, the singular forms “a,” “an,” and “the” include plural referents unless the context clearly dictates otherwise. Thus, for example, reference to “a fragrance ingredient” includes not only a single fragrance ingredient but also a combination or mixture of two or more different fragrance ingredients, reference to “an additive” includes a single additive as well as two or more additives, and the like.

As used herein, the phrases “for example,” “for instance,” “such as,” or “including” are meant to introduce examples that further clarify more general subject matter. These examples are provided only as an aid for understanding the disclosure, and are not meant to be limiting in any fashion. Furthermore, as used herein, the terms “may,” “optional,” “optionally,” or “may optionally” mean that the subsequently described circumstance may or may not occur, so that the description includes instances where the circumstance occurs and instances where it does not. For example, the phrase “optionally present” means that an object may or may not be present, and, thus, the description includes instances wherein the object is present and instances wherein the object is not present.

As used herein, “optionally substituted” means that the indicated core or functional group is either unsubstituted or substituted by one or more groups up to the maximum permitted by the rules of valency, wherein said groups are selected from: halo, hydroxy, cyano, C₁₋₆alkyl, C₂₋₆alkenyl, C₂₋₆alkynyl, C₃₋₆cycloalkyl, C₁₋₆haloalkyl, C₁₋₆-alkoxy, —O—Si(R^{x})₃, —O—R^{x}, —C(O)H, —C(O)—R^{x}, —C(O)—O—R^{x}, —C(O)—NH—R^{x}, —C(O)—N—(R^{x})(R^{x}), —O—C(O)—R^{x}, —NH(R^{x})—C(O)—R^{x}, —N(R^{x})(R^{x})—C(O)—R^{x}), —NH(R^{x}), —N(R^{x})(R^{x}), heterocycloalkyl, aryl, and heteroaryl; wherein each of said C₁₋₆alkyl, C₃₋₆cycloalkyl, heterocycloalkyl, aryl or heteroaryl is further optionally substituted by one or more halo, hydroxy, cyano, C₁₋₆alkyl, C₂₋₆alkenyl, C₂₋₆alkynyl, C₃₋₆cycloalkyl, C₁₋₆haloalkyl, —O—Si(R^{x})₃, —O—R^{x}, —C(O)H, —C(O)—R^{x}, —C(O)—O—R^{x}, —C(O)—NH—R^{x}, —C(O)—N—(R^{x})(R^{x}), —O—C(O)—R^{x}, —NH(R^{x})—C(O)—R^{x}, —N(R^{x})(R^{x})—C(O)—R^{x}), —NH(R^{x}), —N(R^{x})(R^{x}), heterocycloalkyl, aryl, and heteroaryl; and wherein each R^{x} is independently selected from hydrogen, C₁₋₆alkyl, C₂₋₆alkenyl, C₂₋₆alkynyl, C₃₋₆cycloalkyl, heterocycloalkyl, aryl and heteroaryl.

As used herein, the term “C₁₋₆-alkyl” means a saturated linear or branched free radical consisting essentially of 1 to 6 carbon atoms and a corresponding number of hydrogen atoms. Exemplary C₁₋₆-alkyl groups include methyl, ethyl, n-propyl, isopropyl, n-butyl, isobutyl, sec-butyl, tert-butyl, n-pentyl, isopentyl, neopentyl, n-hexyl, and thexyl. Other C₁₋₆-alkyl groups will be readily apparent to those of skill in the art given the benefit of the present disclosure. The terms “C₁₋₃-alkyl”, “C₁₋₄-alkyl”, etc., have equivalent meanings, i.e., saturated linear or branched free radical consisting essentially of 1 to 3 (or 4) carbon atoms and a corresponding number of hydrogen atoms. Exemplary C₁₋₆-alkyl groups include methyl, ethyl, n-propyl, isopropyl, n-butyl, isobutyl, sec-butyl, tert-butyl. The similar terms “C₂₋₆-alkenyl,” “C₂₋₆-alkynyl,” “C₃₋₆-cycloalkyl,” “C₁₋₆-haloalkyl,” “C₁₋₆-alkoxy,” and the like, refer to corresponding functional groups having the stated number of carbon atoms, wherein “alkenyl” refers to an unsaturated linear or branched free radical having at least one double bond, “alkynyl” refers to an unsaturated linear or branched free radical having at least one triple bond, “haloalkyl” refers to an alkyl radical having at least one halogen atom attached to a carbon atom, and “alkoxy” refers to an alkyl radical having at least one oxygen atom attached to the alkyl radical and wherein the attachment point of the functional group is through the oxygen (i.e., to form an ether). Exemplary alkenyl groups include vinyl and allyl. Exemplary alkynyl groups include ethynyl and propynyl. Exemplary haloalkyl groups include fluoromethyl, difluoromethyl, trifluoromethyl, 3,3,3-trifluorethyl, and like groups with chlorine, bromine or iodine. “Cycloalkyl” refers to a carbocyclic ring attached via a ring carbon atom. Exemplary cycloalkyl groups include cyclopropyl, cyclobutyl, cyclopentyl and cyclohexyl.

As used herein, the term “heteroaryl” means an aromatic free radical having 5 to 20 atoms (i.e., ring atoms) that form a ring, wherein at least one atom (e.g., 1 to 5) of the ring atoms are carbon and at least one atom of the remaining ring atoms is a nitrogen, sulfur, or oxygen. Heteroaryl rings include monocyclic, bicyclic fused, and polycyclic fused ring systems provided that at least one ring of the ring system has at least one heteroatom (N, S, or O), and all rings are aromatic. Exemplary 5-membered heteroaryl groups include furyl, thienyl (thiophenyl), pyrrolyl, oxazolyl, thiazolyl, pyrazolyl, isothiazolyl, isoxazolyl, imidazolyl, triazolyl, oxadiazolyl, thiadiazolyl, and tetrazolyl. Exemplary 6-membered heteroaryl groups include pyridyl, pyrimidyl, pyrazinyl, pyridazinyl, and 1,2,4-triazinyl. Exemplary fused heteroaryl groups include benzoxazolyl, benzothiazolyl, benzisothiazolyl, benzisoxazolyl, benzimidazolyl, indolyl, quinolinyl, isoquinolinyl, quinazolinyl, and quinoxalinyl. Other heteroaryl groups will be readily apparent to those of skill in the art given the benefit of the present disclosure. In general, the heteroaryl group typically is attached to the main structure via a carbon atom. However, those of skill in the art will realize that certain other atoms, e.g., hetero ring atoms, can be attached to the main structure.

As used herein, the term “aryl” means an aromatic free radical having 5 or 6 atoms (i.e., ring atoms) that form a ring, wherein all of the ring atoms are carbon. Exemplary aryl groups include phenyl and naphthyl.

As used herein, the term “heterocycloalkyl” means an aromatic free radical having 3 to 20 atoms (i.e., ring atoms) that form a ring, wherein at least one atom (e.g., 1 to 5) of the ring atoms are carbon and at least one atom of the remaining ring atoms is a nitrogen, sulfur, or oxygen, and wherein at least one ring is non-aromatic. Heterocycloalkyl rings include monocyclic, bicyclic fused, bicyclic spiro-joined, polycyclic bridged, and polycyclic fused ring systems, provided that at least one ring of the ring system has at least one heteroatom (N, S, or O) and at least one ring of the ring system is non-aromatic (e.g., saturated). Exemplary saturated heterocycloalkyl groups include azetidinyl, aziridinyl, oxiranyl, oxetanyl, tetrahydrofuranyl, pyrrolidinyl, piperidinyl, piperazinyl, morpholinyl, and thiomorpholinyl. Heterocycloalkyl rings systems include ring systems in which an aromatic ring is fused to a nonaromatic ring, such as will be obtained by partial reduction of a polycyclic aromatic ring system. Exemplary ring systems of this category include indolinyl, tetrahydroquinolinyl, and tetrahydroisoquinolinyl. Other heterocycloalkyl groups will be readily apparent to those of skill in the art given the benefit of the present disclosure. A heterocycloalkyl groups can be attached to the main structure either through a carbon atom or a nitrogen atom of the ring.

The term “compound,” as used herein, unless otherwise indicated, refers to any specific chemical compound disclosed herein and includes tautomers, regioisomers, geometric isomers, and where applicable, stereoisomers, including optical isomers (enantiomers) and other stereoisomers (diastereomers) thereof, as well as pharmaceutically acceptable salts and derivatives thereof where applicable, in context. Within its use in context, the term compound generally refers to a single compound, but also may include other compounds such as stereoisomers, regioisomers and/or optical isomers (including racemic mixtures) as well as specific enantiomers or enantiomerically enriched mixtures of disclosed compounds. The term also refers, in context to prodrug forms of compounds which have been modified to facilitate the administration and delivery of compounds to a site of activity. The term also refers to any specific chemical compound in which one or more atoms have been replaced with one or more different isotopes of the same element.

The term “compound of Formula I” as used herein refers to the compound of Formula IA, IB, IC, ID, IE, IF, or any combination thereof, unless otherwise indicated.

The term “compound of Formula II” as used herein refers to the compound of Formula IIA, IIB, IIC, IID, IIE, IIF, or any combination thereof, unless otherwise indicated.

The entire disclosure of each of the patent documents and scientific articles referred to herein is incorporated by reference in its entirety for all purposes. The function and advantages of these and other embodiments will be more fully understood from the following non-limiting examples. The examples are intended to be illustrative in nature and are not to be considered as limiting the scope of the embodiments discussed herein.

### EXAMPLES

Compounds of the present disclosure can be made according to known methods published in the art.

Ethyl 3-(3-thienyl)propionate is obtained and its olfactory qualities are studied. Both the neat liquid compound and a 10% dilution of the compound in ethanol are examined by an experienced fragrance chemist (a master perfumer). It is found that the compound exhibits a favorable odor profile.

### Synthetic Examples

The Compounds of Examples 1 to 127 may be prepared according to the procedures described hereinbelow.

¹H-NMR spectra were recorded at 400 MHz on a Bruker Avance AV-I-400 instrument or on a Bruker Avance AV-II-400 instrument. Chemical shift values are expressed in ppm-values relative to tetramethylsilane unless noted otherwise. The following abbreviations or their combinations are used for multiplicity of NMR signals: br=broad, d=doublet, m=multiplet, q=quartet, quint=quintet, s=singlet and t=triplet.

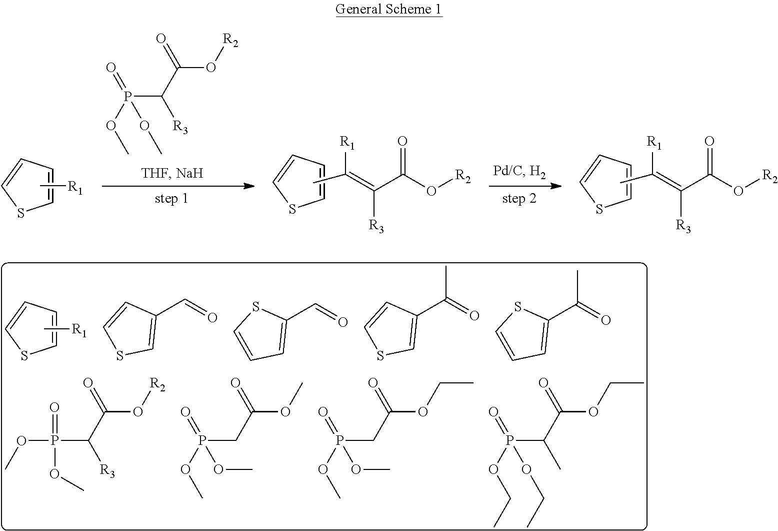

### Example 1

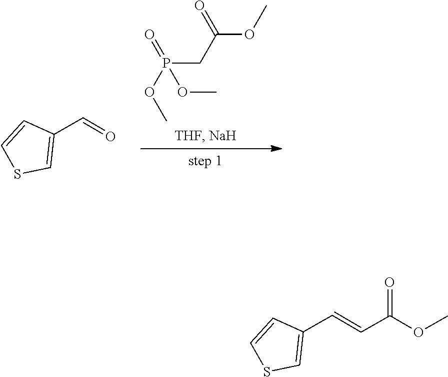

A solution of methyl 2-(dimethoxyphosphoryl) acetate (5.85 g, 32.10 mmol, 1.20 equiv) in THF (60 Ml was treated with NaH (1.28 g, 53.50 mmol, 2.00 equiv) at 0° C. for 30 min under air atmosphere followed by the addition of 3-thiophenecarboxaldehyde (3.00 g, 26.75 mmol, 1.00 equiv) in portions at room temperature for 3 h. The reaction was quenched with sat. NH₄Cl (aq.) at room temperature. The resulting mixture was extracted with EtOAc (20 mL). The combined organic layers were washed with water (3×20 mL), dried over anhydrous Na₂SO₄. After filtration, the filtrate was concentrated under reduced pressure. The residue was purified by silica gel column chromatography, eluted with PE/EA (10:1) to afford methyl (2E)-3-(thiophen-3-yl) prop-2-enoate (2.68 g, 98.44% purity, 59.6% yield) as a colorless oil.

The following Examples were synthesized in a similar manner to that described for Example 1.

| | | GC-MS | |
|---|---|---|---|
| | | (EI+, m/z, | |
| Number | Structure | M+) | ¹H-NMR |
| Example 2 | 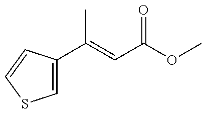 | 182.0 | ¹H-NMR (400 MHz, Chloroform-d, ppm) δ 8.14 (dd, J = 3.0, 1.3 Hz, 1H), 7.58 (dd, J = 5.1, 1.3 Hz, 1H), 7.40-7.33 (m, 1H), 3.93 (s, 2H), 3.78 (s, 3H) |
| Example 3 | 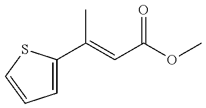 | 182.1 | ¹H-NMR (300 MHz, Chloroform-d, ppm) δ 7.46-7.25 (m, 2H), 7.12-7.02 (m, 1H), 6.29 (q, J = 1.2 Hz, 1H), 3.74 (d, J = 15.5 Hz, 3H), 2.64 (d, J = 1.3 Hz, 3H) |
| Example 4 | 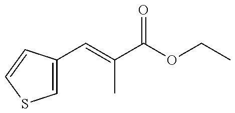 | 196.1 | ¹H-NMR (400 MHz, Chloroform-d, ppm) δ 7.71-7.66 (m, 1H), 7.51-7.45 (m, 1H), 7.41-7.34 (m, 1H), 7.30-7.26 (m, 1H), 4.29 (q, J = 7.1 Hz, 2H), 2.21-2.16 (m, 3H), 1.37 (t, J = 7.1 Hz, 3H). |
| Example 5 | 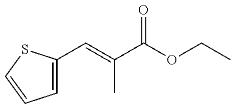 | 196.1 | ¹H-NMR (400 MHz, Chloroform-d, ppm) δ 7.88 (d, J = 1.5 Hz, 1H), 7.52 (d, J = 5.1 Hz, 1H), 7.31 (d, J = 3.7 Hz, 1H), 7.14 (dd, J = 5.1, 3.7 Hz, 1H), 4.29 (q, J = 7.1 Hz, 2H), 2.24 (d, J = 1.3 Hz, 3H), 1.37 (t, J = 7.1 Hz, 3H). |
| Example 121 | 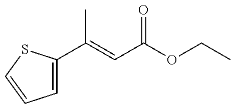 | 196.1 | ¹H-NMR (300 MHz, Chloroform-d, ppm): δ 7.30-7.32 (m, 2H), 7.04 (mc, 1H), 6.25 (q, J = 1.2 Hz, 1H), 4.20 (q, J = 7.1 Hz, 2H), 2.60 (d, J = 1.2 Hz, 3H), 1.31 (t, J = 7.1 Hz, 3H). |

### Example 6

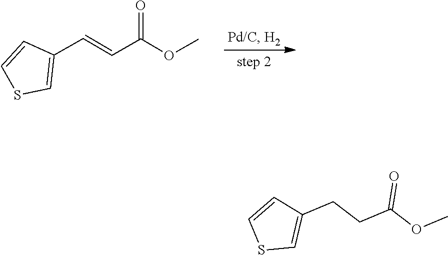

A solution of methyl (2E)-3-(thiophen-3-yl) prop-2-enoate (2.68 g, 15.93 mmol, 1.00 equiv) and Pd/C (300.00 mg) in MeOH (30 mL was stirred at room temperature for overnight under hydrogen atmosphere. The resulting mixture was filtered; the filter cake was washed with ethyl acetate (3×20 mL). The filtrate was concentrated under reduced pressure. The residue was purified by silica gel column chromatography, eluted with PE/LA (9:1) to afford methyl 3-(thiophen-3-yl) propanoate (2.51 g, 99.83% purity, 92.9% yield) as a colorless oil. GC-MS: EI+, m/z, M⁺: 170.0. ¹H-NMR (300 MHz, Chloroformn-d, ppm) δ 7.28 (dd, J=4.8, 3.0 Hz, 1H), 7.04-6.93 (m, 2H), 3.70 (s, 3H), 3.06-2.95 (m, 2H), 2.66 (dd, J=8.3, 7.1 Hz, 2H).

The following Examples were synthesized in a similar manner to that described for Example 6:

| | | GC-MS | |
|---|---|---|---|
| | | (EI+, m/z, | |
| Number | Structure | M+) | ¹H-NMR |
| Example 7 | 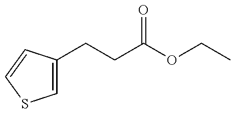 | 184.0 | ¹H-NMR (300 MHz, Chloroform-d, ppm) δ 7.28 (dd, J = 4.8, 2.7 Hz, 1H), 6.99 (dd, J = 8.9, 3.9 Hz, 2H), 4.16 (q, J = 7.1 Hz, 2H), 3.00 (t, J = 7.7 Hz, 2H), 2.65 (t, J = 7.7 Hz, 2H), 1.27 (t, J = 7.1 Hz, 1H) |
| Example 8 | 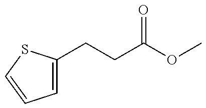 | 170.1 | ¹H-NMR (300 MHz, Chloroform-d, ppm) δ 7.15 (dd, J = 5.1, 1.2 Hz, 1H), 6.94 (dd, J = 5.2, 3.4 Hz, 1H), 6.85 (dq, J = 3.3, 1.0 Hz, 1H), 3.72 (s, 3H), 3.19 (ddd, J = 7.8, 7.1, 0.9 Hz, 2H), 2.72 (dd, J = 8.2, 7.0 Hz, 2H) |
| Example 9 | 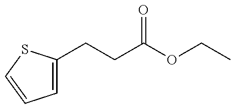 | 184.1 | ¹H-NMR (300 MHz, DMSO-d₆, ppm) δ 4.05 (s, 1H), 2.21 (d, J = 11.8 Hz, 2H), 1.86-1.45 (m, 10H), 1.43-1.28 (m, 2H), 1.20 (s, 3H). |
| Example 10 | 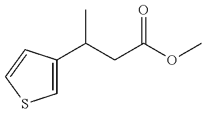 | 184.1 | ¹H-NMR (300 MHz, Chloroform-d, ppm) δ 7.37-7.24 (m, 1H), 7.01 (d, J = 4.6 Hz, 2H), 3.67 (s, 3H), 3.43 (q, J = 7.2 Hz, 1H), 2.73- 2.46 (m, 2H), 1.33 (d, J = 6.9 Hz, 3H). |
| Example 11 | 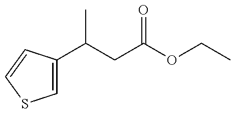 | 184.0 | ¹H-NMR (300 MHz, Chloroform-d, ppm) δ 7.32-7.24 (m, 1H), 7.01 (d, J = 4.0 Hz, 2H), 4.13 (q, J = 7.1 Hz, 2H), 3.41 (dt, J = 14.2, 7.1 Hz, 1H), 2.64 (dd, J = 15.0, 6.8 Hz, 1H), 2.51 (dd, J = 15.0, 8.1 Hz, 1H), 1.33 (s, 1H), 1.33 (d, J = 6.9 Hz, 3H), 1.23 (t, J = 7.1 Hz, 3H). |
| Example 12 | 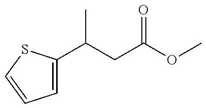 | 198.1 | ¹H-NMR (300 MHz, Chloroform-d, ppm) δ 7.16 (dd, J = 5.1, 1.2 Hz, 1H), 6.94 (dd, J = 5.1, 3.5 Hz, 1H), 6.86 (dt, J = 3.5, 1.0 Hz, 1H), 3.69 (s, 3H), 3.73-3.53 (m, 1H), 2.72 (dd, J = 15.4, 6.7 Hz, 1H), 2.59 (dd, J = 15.3, 8.0 Hz, 1H), 1.41 (d, J = 6.9 Hz, 3H). |
| Example 13 | 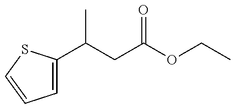 | 198.1 | ¹H-NMR (300 MHz, Chloroform-d, ppm) δ 7.16 (dd, J = 5.1, 1.2 Hz, 1H), 6.94 (dd, J = 5.1, 3.5 Hz, 1H), 6.86 (dt, J = 3.5, 1.0 Hz, 1H), 4.15 (q, J = 7.1 Hz, 2H), 3.71-3.56 (m, 1H), 2.71 (dd, J = 15.2, 6.8 Hz, 1H), 2.57 (dd, J = 15.2, 8.0 Hz, 1H), 1.41 (d, J = 6.9 Hz, 3H), 1.25 (t, J = 7.1 Hz, 3H). |
| Example 14 | 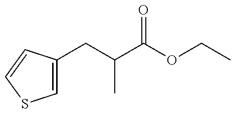 | 198.1 | ¹H-NMR (400 MHz, Chloroform-d, ppm) δ 7.26 (dd, J = 4.9, 2.9 Hz, 1H), 6.99 (dt, J = 3.1, 1.1 Hz, 1H), 6.94 (dd, J = 4.9, 1.3 Hz, 1H), 4.13 (q, J = 7.1 Hz, 2H), 3.10-2.99 (m, 1H), 2.74 (dqt, J = 10.3, 6.9, 3.0 Hz, 2H), 1.33-1.13 (m, 6H). |
| Example 15 | 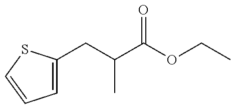 | 198.1 | ¹H-NMR (400 MHz, Chloroform-d, ppm) δ 7.15 (dd, J = 5.1, 1.2 Hz, 1H), 6.93 (dd, J = 5.1, 3.4 Hz, 1H), 6.83 (dq, J = 3.3, 1.0 Hz, 1H), 4.15 (q, J = 7.1 Hz, 2H), 3.24 (ddd, J = 14.7, 7.2, 0.9 Hz, 1H), 2.95 (ddd, J = 14.7, 7.2, 0.9 Hz, 1H), 2.77 (h, J = 7.1 Hz, 1H), 1.33-1.16 (m, 6H). |

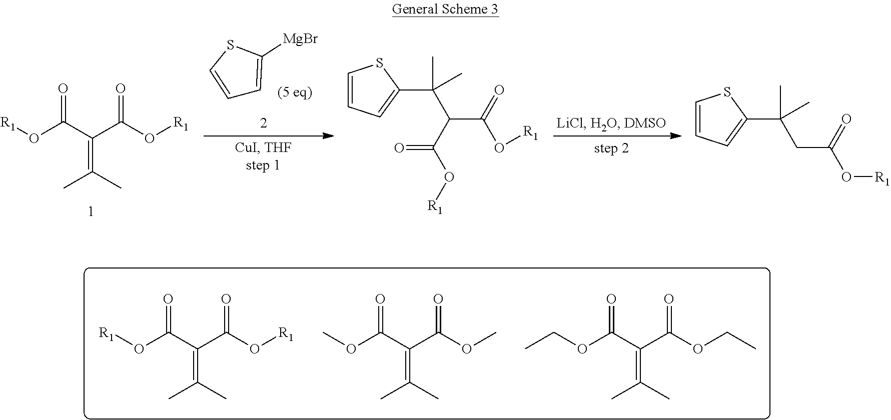

### Example 16

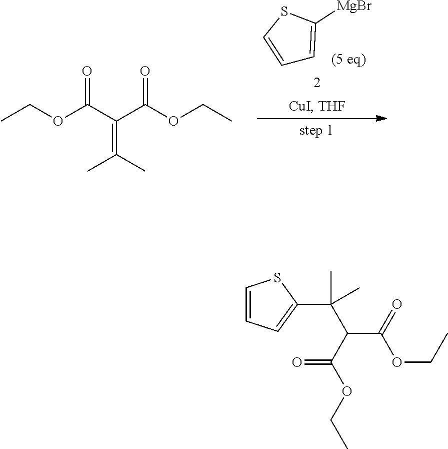

Step 1: A solution of CuI (19.02 g, 99.88 mmol, 2.00 equiv) in THF (100 mL) was treated with 2-(bromomagnesium)thiophene (1M, 249.7 mL, 5.00 equiv) at 0° C. for 30 min under nitrogen atmosphere followed by the addition of 1,3-diethyl 2-(propan-2-ylidene) propanedioate (10 g, 49.94 mmol, 1.00 equiv) dropwise at 0° C. The resulting mixture was stirred at room temperature for overnight under nitrogen atmosphere. The reaction was quenched with sat. NH₄Cl (aq.) at 0° C. The resulting mixture was extracted with EtOAc (100 mL). The combined organic layer was washed with water (3×100 mL), dried over anhydrous Na₂SO₄. After filtration, the filtrate was concentrated under reduced pressure. The residue was purified by silica gel column chromatography, eluted with PE/THF (10:1) to afford 1,3-diethyl 2-[2-(thiophen-2-yl)propan-2-yl]propanedioate (4.8 g, 33.80% yield, 80% purity) as a green oil.

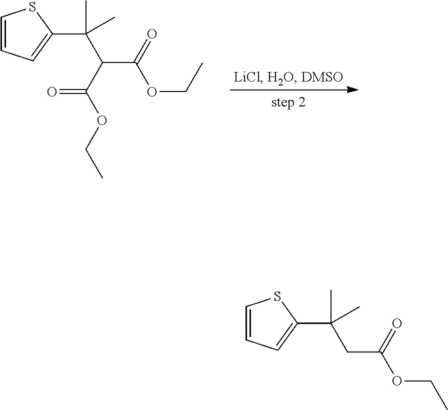 [SMILES: CCOC(=O)C(C(=O)OCC)C(C)(C)c1cccs1 ; CCOC(=O)CC(C)(C)c1cccs1]

Step 2: A solution of 1,3-diethyl 2-[2-(thiophen-2-yl)propan-2-yl]propanedioate (4.8 g, 16.88 mmol, 1.00 equiv), LiCl (3.58 g, 84.40 mmol, 5.00 equiv) and H₂O (0.30 g, 16.88 mmol, 1.00 equiv) in DMSO (48 mL) was stirred at 160° C. for 5 h under nitrogen atmosphere. The mixture was allowed to cool down to room temperature. The reaction was quenched with water at room temperature. The resulting mixture was extracted with EtOAc (50 mL). The combined organic layer was washed with water (3×50 mL), dried over anhydrous Na₂SO₄. After filtration, the filtrate was concentrated under reduced pressure. The residue was purified by silica gel column chromatography, eluted with PE/THF (10:1) to afford ethyl 3-methyl-3-(thiophen-2-yl)butanoate (505.7 mg, 14.11% yield, 98.3% purity) as a yellow oil. GC-MS: (EI+, m/z, M+): 212.1. 1H NMR (300 MHz, Chloroform-d, ppm) δ 7.17 (dd, J=5.1, 1.2 Hz, 1H), 6.98-6.84 (m, 2H), 4.07 (q, J=7.1 Hz, 2H), 2.65 (s, 2H), 1.55 (s, 6H), 1.18 (t, J=7.1 Hz, 3H).

The following Example was synthesized in a similar manner to that described for Example 16:

| | | GC-MS | |
|---|---|---|---|
| | | (EI+, | |
| Num- | | m/z, | |
| ber | Structure | M⁺) | ¹H-NMR |
| Ex- am- ple 17 | 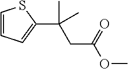 | 198.1000 | ¹H NMR (300 MHz, DMSO- d₆, ppm) δ 7.33 (dd, J = 4.9, 1.4 Hz, 1H), 6.98-6.87 (m, 2H), 3.51 (s, 3H), 2.65 (s, 2H), 1.44 (s, 6H). |

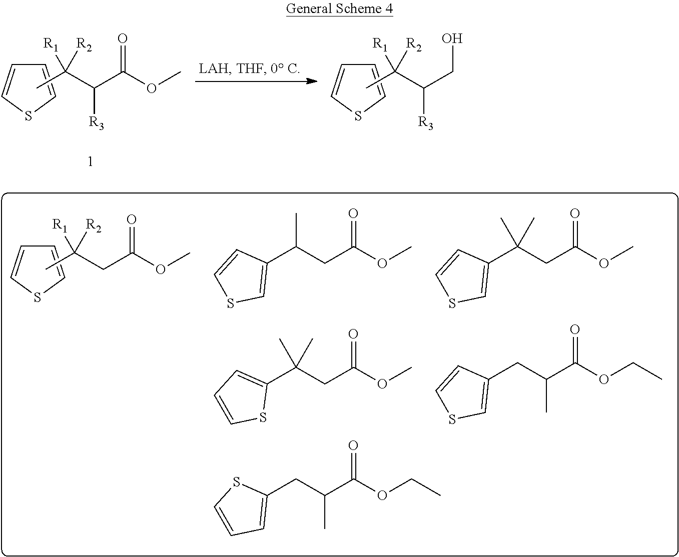

### Example 18

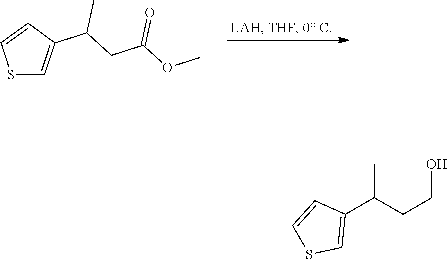

A solution of methyl 3-(thiophen-3-yl) butanoate (2.00 g, 10.85 mmol, 1.00 equiv) in THF (17 mL) was treated with LAH (0.49 g, 13.02 mmol, 1.20 equiv) at 0° C. for 5 h. The reaction was quenched by the addition of MeOH (20 mL) at 0° C. The resulting mixture was extracted with EtOAc (3×30 mL). The combined organic layers were washed with water (3×30 mL). dried over anhydrous Na₂SO₄. After filtration, the filtrate was concentrated under reduced pressure. The residue was purified by silica gel column chromatography, eluted with PE/THF (8:1) to afford 3-(thiophen-2-yl) butan-1-ol (984.2 mg, 97.82% purity, 58.2% yield) as a colorless oil.

The following Examples were synthesized in a similar manner to that described for Example 18:

| | | LC-MS | | |
|---|---|---|---|---|
| | | (ES+, | GC-MS | |
| | | m/z, | (EI+, m/z, | |
| Number | Structure | [M + H]⁺) | M⁺) | ¹H-NMR |
| Example 19 | 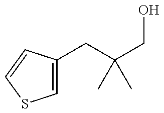 | / | 170.1 | ¹H-NMR (300 MHz, Chloroform-d, ppm) δ 7.25 (dd, J = 4.8, 3.0 Hz, 1H), 7.04- 6.91 (m, 2H), 3.34 (s, 2H), 2.63 (s, 2H), 0.92 (s, 6H). |
| Example 20 | 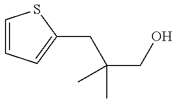 | / | 170.1 | ¹H-NMR (300 MHz, Chloroform-d, ppm) δ 7.16 (dd, J = 5.1, 1.2 Hz, 1H), 6.97 (dd, J = 5.1, 3.4 Hz, 1H), 6.82 (ddt, J = 3.4, 1.3, 0.7 Hz, 1H), 3.38 (s, 2H), 2.83 (d, J = 0.7 Hz, 2H), 1.52 (s, 2H), 0.97 (s, 6H). |
| Example 21 | 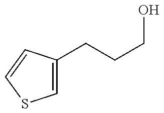 | / | 142.0 | ¹H NMR (300 MHz, Chloroform-d, ppm) δ 7.32-7.25 (m, 1H), 7.02-6.94 (m, 2H), 3.71 (t, J = 6.4 Hz, 2H), 2.82-2.71 (m, 2H), 2.06-1.85 (m, 2H). |
| Example 22 | 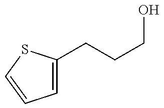 | / | 142.1 | ¹H NMR (300 MHz, Chloroform-d, ppm) δ 7.14 (dd, J = 5.2, 1.2 Hz, 1H), 6.94 (dd, J = 5.1, 3.4 Hz, 1H), 6.83 (dq, J = 3.3, 1.0 Hz, 1H), 3.74 (t, J = 6.3 Hz, 2H), 2.97 (t, J = 7.6 Hz, 2H), 1.97 (dq, J = 7.5, 6.4 Hz, 2H). |
| Example 23 | 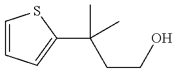 | 171.1 | / | ¹H NMR (300 MHz, DMSO-d₆, ppm) δ 7.32 (dd, J = 5.1, 1.2 Hz, 1H), 6.93 (dd, J = 5.1, 3.5 Hz, 1H), 6.85 (dd, J = 3.5, 1.2 Hz, 1H), 4.30 (t, J = 5.1 Hz, 1H), 3.38- 3.25 (m, 2H), 1.85-1.73 (m, 2H), 1.33 (s, 6H). |
| Example 24 | 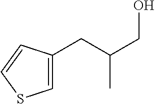 | / | 156.1 | ¹H NMR (400 MHz, Chloroform-d, ppm) δ 7.28 (dd, J = 4.9, 3.0 Hz, 1H), 6.97 (td, J = 4.7, 1.2 Hz, 2H), 3.53 (qd, J = 10.6, 6.0 Hz, 2H), 2.78 (dd, J = 14.0, 6.3 Hz, 1H), 2.53 (dd, J = 14.1, 7.8 Hz, 1H), 2.06-1.92 (m, 1H), 0.96 (d, J = 6.8 Hz, 3H). |
| Example 25 | 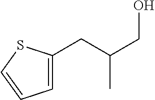 | / | 156.1 | ¹H NMR (400 MHz, Chloroform-d, ppm) δ 7.16 (dd, J = 5.1, 1.2 Hz, 1H), 6.95 (dd, J = 5.1, 3.4 Hz, 1H), 6.82 (dt, J = 3.2, 1.0 Hz, 1H), 3.62-3.48 (m, 2H), 2.99 (ddd, J = 14.6, 6.4, 0.9 Hz, 1H), 2.72 (ddd, J = 14.6, 7.8, 0.8 Hz, 1H), 2.00 (ddt, J = 14.2, 7.1, 6.1 Hz, 1H), 1.00 (d, J = 6.8 Hz, 3H). |

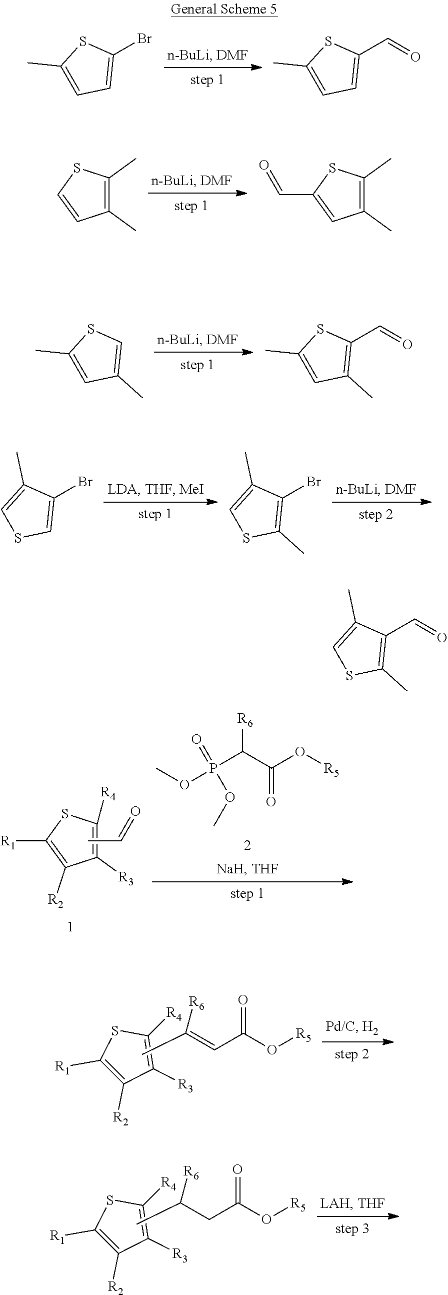

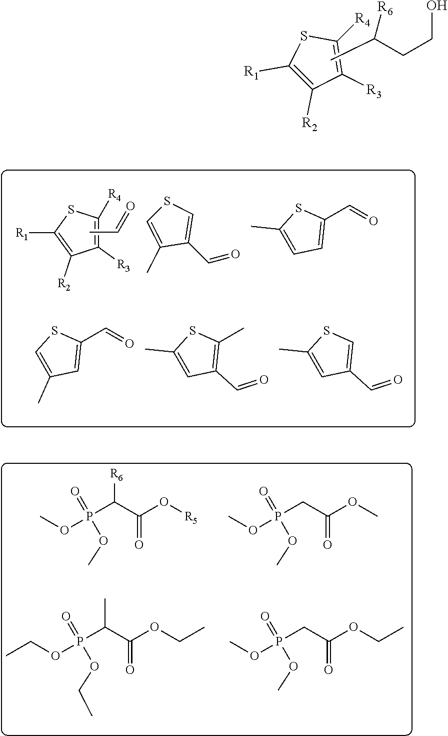

### Example 26

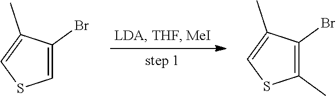

Step 1: A solution of 3-bromo-4-methylthiophene (10 g, 56.48 mmol, 1.00 equiv) in THF (100 mL) was treated with LDA (7.87 g, 73.42 mmol, 1.30 equiv) at −40° C. for 1 h under nitrogen atmosphere followed by the addition of CH₃I (10.42 g, 73.42 mmol, 1.30 equiv) dropwise at −40° C. The resulting mixture was stirred at room temperature for overnight under nitrogen atmosphere. The reaction was quenched with sat. NH₄Cl (aq.) at room temperature. The resulting mixture was extracted with EtOAc (50 mL). The combined organic layer was washed with water (3×50 mL), dried over anhydrous Na₂SO₄. After filtration, the filtrate was concentrated under reduced pressure. The residue was purified by silica gel column chromatography, eluted with PE/THF (10:1) to afford 3-bromo-2,4-dimethylthiophene (6 g, 55.59% yield, 85% purity) as an orange oil.

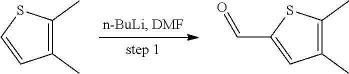

Step 2: A solution of thiophene, 2,3-dimethylthiophene (12 g, 106.96 mmol, 1.00 equiv) in THF (100 mL) was treated with n-BuLi (20.56 g, 320.88 mmol, 3.00 equiv) at −78° C. for 1 h under nitrogen atmosphere, followed by the addition of DMF (78.21 g, 1069.62 mmol, 10.00 equiv) dropwise at 0° C. The resulting mixture was stirred at 60° C. for 1 h under nitrogen atmosphere. The reaction was quenched with sat. NH₄Cl (aq.) at room temperature. The resulting mixture was extracted with EtOAc (50 mL). The combined organic layers were washed with water (3×50 mL), dried over anhydrous Na₂SO₄. After filtration, the filtrate was concentrated under reduced pressure. The residue was purified by silica gel column chromatography, eluted with PE/THF (12:1) to afford 4,5-dimethylthiophene-2-carbaldehyde (12.34 g, 95.44% purity, 82.27% yield) as a yellow oil.

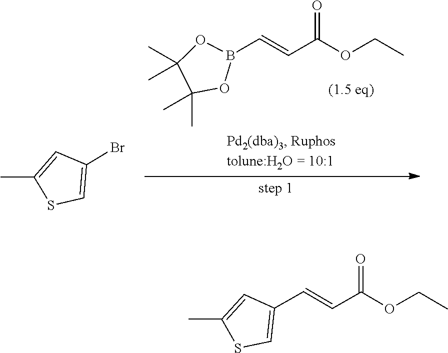

Step 3: A solution of 4-bromo-2-methylthiophene (10 g, 56.48 mmol, 1.00 equiv) in H₂O (10 mL) and toluene (100 mL) was treated with ethyl (2E)-3-(4,4,5,5-tetramethyl-1,3,2-dioxaborolan-2-yl) prop-2-enoate (19.15 g, 84.72 mmol, 1.50 equiv) and RuPhos (5.27 g, 11.30 mmol, 0.20 equiv) at room temperature for 3 min under nitrogen atmosphere followed by the addition of Pd₂(dba)₃ (5.17 g, 5.65 mmol, 0.10 equiv) in portions at room temperature. The resulting mixture was stirred at 80° C. for overnight under nitrogen atmosphere. The reaction was quenched with water at room temperature. The resulting mixture was extracted with EtOAc (50 mL). The combined organic layers were washed with water (3×50 mL), dried over anhydrous Na₂SO₄. After filtration, the filtrate was concentrated under reduced pressure. The residue was purified by silica gel column chromatography, eluted with PE/THF (10:1) to afford ethyl (2E)-3-(5-methylthiophen-3-yl) prop-2-enoate (6.2 g, 55.93% yield, 85% purity) as a yellow oil. LC-MS ES+, m/z, [M+H]⁺: 197.1. ¹H NMR (400 MHz, Chloroform-d, ppm) δ 7.59 (d, J=15.9 Hz, 1H), 7.27 (s, 1H), 6.97 (s, 1H), 6.20 (d, J=15.9 Hz, 1H), 4.26 (q, J=7.1 Hz, 2H), 2.50 (s, 3H), 1.35 (t, J=7.1 Hz, 3H).

### Example 27

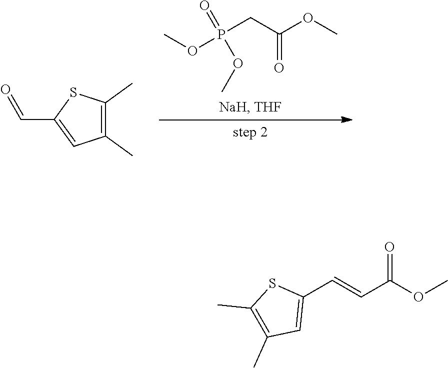

A solution of methyl 2-(dimethoxyphosphoryl) acetate (10 g, 67.76 mmol, 1.00 equiv) in THF (120 mL) was treated with NaH (2.1 g, 135.52 mmol, 2.00 equiv) at 0° C. for 30 min under nitrogen atmosphere followed by the addition of 4,5-dimethylthiophene-2-carbaldehyde (5 g, 67.76 mmol, 1.00 equiv) in portions at 0° C. The resulting mixture was stirred at room temperature for additional 16 h. The resulting mixture was extracted with EtOAc (30 mL). The combined organic layers were washed with water (3×30 mL), dried over anhydrous Na₂SO₄. After filtration, the filtrate was concentrated under reduced pressure. The residue was purified by silica gel column chromatography, eluted with PE/EA (10:1) to afford methyl (2E)-3-(4,5-dimethylthiophen-2-yl) prop-2-enoate (5 g, 95.21% purity, 71.43% yield) as a yellow solid. GC-MS: EI+, m/z, M⁺: 196.1. ¹H-MR (300 MHz, Chloroform-d, ppm) δ 7.68 (d, J=15.6 Hz, 1H), 6.97 (s, 1H), 6.10 (d, J=15.6 Hz, 1H), 3.79 (s, 3H), 2.37 (s, 3H), 2.13 (s, 3H).

The following Examples were synthesized in a similar manner to that described for Examples 26 and 27:

| | | LC-MS | | |
|---|---|---|---|---|
| | | (ES+, | GC-MS | |
| | | m/z, | (EI+, m/z, | |
| Number | Structure | [M + H]⁺) | [M+]⁺) | ¹H-NMR |
| Example 28 | 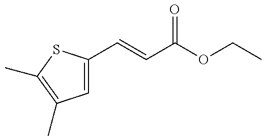 | 211.1 | / | ¹H-NMR (300 MHz, Chloroform-d, ppm) δ 7.67 (d, J = 15.6 Hz, 1H), 6.96 (s, 1H), 6.09 (d, J = 15.6 Hz, 1H), 4.25 (q, J = 7.2 Hz, 2H), 2.37 (s, 3H), 2.13 (s, 3H), 1.34 (t, J = 7.1 Hz, 4H). |
| Example 29 | 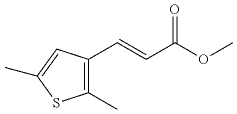 | / | 196.0 | ¹H-NMR (300 MHz, Chloroform-d, ppm) δ 7.65 (d, J = 15.8 Hz, 1H), 6.84 (d, J = 1.4 Hz, 1H), 6.13 (d, J = 15.8 Hz, 1H), 3.81 (s, 3H), 2.49 (s, 3H), 2.46-2.39 (m, 3H). |
| Example 30 | 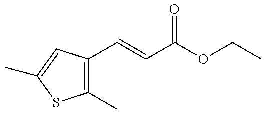 | / | 210.1 | ¹H-NMR (300 MHz, Chloroform-d, ppm) δ 7.64 (d, J = 15.8 Hz, 1H), 6.83 (d, J = 1.4 Hz, 1H), 6.13 (d, J = 15.8 Hz, 1H), 4.26 (q, J = 7.1 Hz, 2H), 2.49 (s, 3H), 2.42 (s, 3H), 1.34 (t, J = 7.1 Hz, 3H). |
| Example 31 | 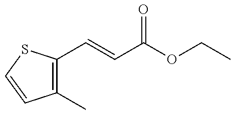 | / | 196.1 | ¹H-NMR (300 MHz, Chloroform-d, ppm) δ 7.87 (d, J = 15.6, 0.9 Hz, 1H), 7.26 (s, 1H), 6.89 (d, J = 5.1 Hz, 1H), 6.21 (dd, J = 15.6, 5.8 Hz, 1H), 4.27 (d, J = 7.1, 2.8 Hz, 1H), 2.37 (s, 2H), 2.27 (d, J = 1.1 Hz, 3H), 1.35 (t, J = 7.1, 2.0 Hz, 3H). |
| Example 32 | 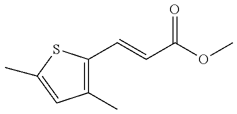 | / | 196.1 | ¹H-NMR (400 MHz, Chloroform-d, ppm) δ 7.79 (d, J = 15.5, 1.6 Hz, 1H), 6.54 (t, J = 1.3 Hz, 1H), 6.02 (dd, J = 15.5, 1.6 Hz, 1H), 3.77 (d, J = 1.6 Hz, 3H), 2.44 (d, J = 1.1 Hz, 3H), 2.26 (d, J = 1.6 Hz, 3H). |
| Example 33 | 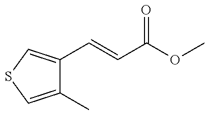 | / | 182.1 | ¹H-NMR (300 MHz, Chloroform-d, ppm) δ 7.69 (d, J = 16.0 Hz, 1H), 7.58 (d, J = 3.2 Hz, 1H), 6.99 (td, J = 2.1, 1.1 Hz, 1H), 6.32 (d, J = 16.0 Hz, 1H), 3.82 (s, 3H), 2.36 (d, J = 1.1 Hz, 3H). |
| Example 34 | 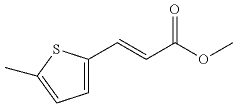 | / | 182.0 | ¹H-NMR (300 MHz, Chloroform-d, ppm) δ 7.73 (d, J = 15.6 Hz, 1H), 7.07 (d, J = 3.6 Hz, 1H), 6.77-6.70 (m, 1H), 6.12 (d, J = 15.6 Hz, 1H), 3.80 (s, 3H), 2.52 (d, J = 1.1 Hz, 3H). |
| Example 35 | 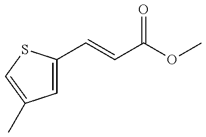 | / | 182.1 | ¹H-NMR (300 MHz, Chloroform-d, ppm) δ 6.71 (p, J = 1.2 Hz, 1H), 6.68-6.61 (m, 1H), 3.73 (t, J = 6.3 Hz, 2H), 2.96-2.85 (m, 2H), 2.23 (d, J = 1.1 Hz, 3H), 2.03-1.85 (m, 2H), 1.44 (s, 1H). |
| Example 36 | 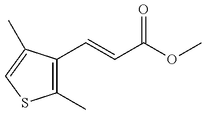 | / | 196.1 | ¹H-NMR (400 MHz, Chloroform-d, ppm) δ 7.74 (d, J = 16.3 Hz, 1H), 6.72 (d, J = 1.2 Hz, 1H), 6.20 (d, J = 16.3 Hz, 1H), 3.83 (s, 3H), 2.57 (s, 3H), 2.33 (d, J = 1.1 Hz, 3H). |
| Example 37 | 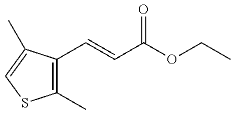 | 211.1 | / | ¹H-NMR (300 MHz, DMSO-d₆, ppm) δ 7.61 (d, J = 16.3 Hz, 1H), 7.00 (d, J = 1.2 Hz, 1H), 6.19 (d, J = 16.3 Hz, 1H), 4.19 (q, J = 7.1 Hz, 2H), 2.51 (s, 3H), 2.26 (d, J = 1.1 Hz, 3H), 1.26 (t, J = 7.1 Hz, 3H). |
| Example 38 | 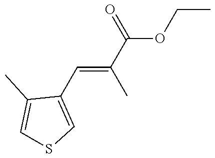 | / | 210.1 | ¹H-NMR (300 MHz, DMSO-d₆, ppm) δ 7.73-7.31 (m, 1H), 7.49-7.12 (m, 1H), 7.29-6.57 (m, 1H), 4.28- 4.01 (m, 2H), 2.23-2.12 (m, 3H), 2.09-2.00 (m, 3H), 1.32-1.05 (m, 3H). |
| Example 39 | 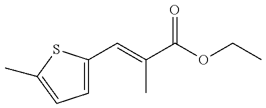 | / | 210.1 | ¹H-NMR (300 MHz, Chloroform-d, ppm) δ 7.78 (t, J = 1.1 Hz, 1H), 7.11 (d, J = 3.6 Hz, 1H), 6.79 (dq, J = 3.4, 1.1 Hz, 1H), 4.28 (q, J = 7.1 Hz, 2H), 2.56 (d, J = 1.1 Hz, 3H), 2.20 (d, J = 1.3 Hz, 3H), 1.36 (t, J = 7.1 Hz, 3H). |
| Example 40 | 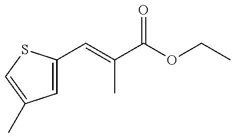 | / | 210.1 | ¹H-NMR (400 MHz, Chloroform-d, ppm) δ 7.80 (d, J = 1.6 Hz, 1H), 7.10 (t, J = 2.5 Hz, 2H), 4.28 (q, J = 7.1 Hz, 2H), 2.30 (d, J = 1.0 Hz, 3H), 2.21 (d, J = 1.4 Hz, 3H), 1.36 (t, J = 7.1 Hz, 3H). |
| Example 41 | 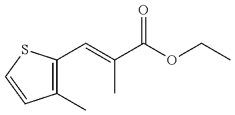 | / | 210.1 | 1H-NMR (300 MHz, Chloroform- d, ppm) δ 8.01-7.75 (m, 1H), 7.49- 7.38 (m, 1H), 7.14-6.88 (m, 1H), 4.42-4.19 (m, 2H), 2.42-2.28 (m, 3H), 2.26-2.19 (m, 3H), 1.41- 1.32 (m, 3H). |

### Example 42

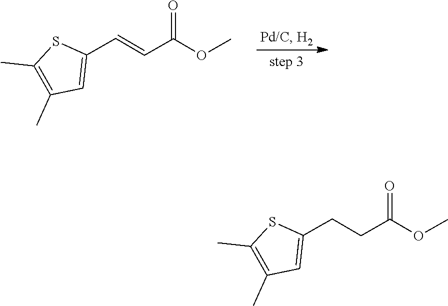

A solution of methyl (E)-3-(4,5-dimethylthiophen-2-yl)acrylate (3 g, 40.76 mmol, 1.00 equiv) and Pd/C (0.87 g, 8.15 mmol, 0.20 equiv) in MeOH (80 mL) was stirred at room temperature for overnight under nitrogen atmosphere. The resulting mixture was filtered; the filter cake was washed with MeOH (3×30 mL). The resulting mixture was concentrated under reduced pressure. The residue was purified by silica gel column chromatography, eluted with PE/LA (10:1) to afford methyl 3-(4,5-dimethylthiophen-2-yl) propanoate (2.9 g, 97.43% purity, 95.71% yield) as a yellow oil. LC-MS: ES+, m/z, [M+H]⁺: 199.1. 1H-NMR (300 MHz, Chloroform-d, ppm) δ 6.51 (s, 1H), 3.71 (s, 3H), 3.06 (t, J=7.6 Hz, 2H), 2.66 (dd, Jp=8.2, 7.0 Hz, 2H), 2.30 (s, 3H), 2.08 (d, J=0.8 Hz, 3H).

The following Examples were synthesized in a similar manner to that described for Example 42:

| | | LC-MS | GC-MS | |
|---|---|---|---|---|
| | | (ES+, m/z, | (EI+, | |
| Number | Structure | [M + H]⁺) | m/z, [M]⁺ | ¹H-NMR |
| Example 43 | 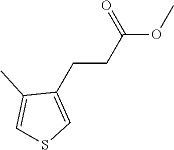 | / | 184.1 | ¹H-NMR (300 MHz, Chloroform-d, ppm) δ 6.93 (s, 2H), 3.72 (s, 3H), 2.94- 2.82 (m, 2H), 2.72-2.60 (m, 2H), 2.22 (d, J = 0.7 Hz, 3H). |
| Example 44 | 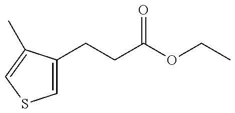 | / | 198.0 | ¹H-NMR (300 MHz, Chloroform-d, ppm) δ 6.65-6.52 (m, 2H), 3.71 (s, 3H), 3.10 (t, J = 7.6 Hz, 2H), 2.68 (dd, J = 8.2, 7.0 Hz, 2H), 2.45 (d, J = 1.1 Hz, 3H). |
| Example 45 | 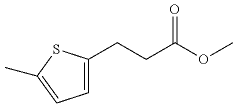 | / | 184.1 | ¹H-NMR (400 MHz, Chloroform-d, ppm) δ 6.97-6.90 (m, 2H), 4.17 (q, J = 7.1 Hz, 2H), 2.88 (dd, J = 9.0, 6.7 Hz, 2H), 2.65 (dd, J = 9.0, 6.6 Hz, 2H), 2.22 (d, J = 0.9 Hz, 3H), 1.28 (t, J = 7.1 Hz, 3H).. |
| Example 46 | 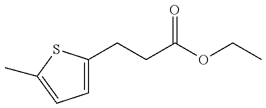 | / | 198.0 | ¹H-NMR (300 MHz, Chloroform-d, ppm) δ 6.64-6.52 (m, 2H), 4.17 (q, J = 7.1 Hz, 2H), 3.10 (t, J = 7.6 Hz, 2H), 2.66 (dd, J = 8.3, 6.9 Hz, 2H), 2.45 (d, J = 1.1 Hz, 3H), 1.28 (t, J = 7.1 Hz, 3H). |
| Example 47 | 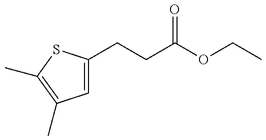 | 213.1 | / | ¹H-NMR (300 MHz, Chloroform-d, ppm) δ 6.51 (s, 1H), 4.17 (q, J = 7.1 Hz, 2H), 3.05 (t, J = 7.6 Hz, 2H), 2.64 (dd, J = 8.3, 7.0 Hz, 2H), 2.30 (s, 3H), 2.08 (d, J = 0.8 Hz, 3H), 1.28 (t, J = 7.1 Hz, 3H). |
| Example 48 | 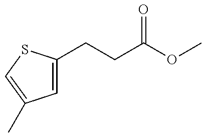 | / | 184.1 | ¹H-NMR (300 MHz, Chloroform-d, ppm) δ 6.71 (p, J = 1.2 Hz, 1H), 6.68- 6.62 (m, 1H), 3.72 (s, 3H), 3.13 (d, J = 7.8, 7.0, 0.9 Hz, 2H), 2.69 (dd, J = 8.2, 7.0 Hz, 2H), 2.22 (d, J = 1.1 Hz, 3H). |
| Example 49 | 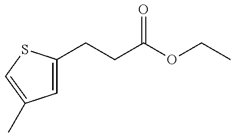 | / | 198.1 | ¹H-NMR (300 MHz, Chloroform-d, ppm) δ 6.71 (p, J = 1.1 Hz, 1H), 6.65 (d, J = 1.4 Hz, 1H), 4.17 (q, J = 7.1 Hz, 2H), 3.12 (d, J = 7.9, 7.0, 0.9 Hz, 2H), 2.68 (dd, J = 8.3, 7.0 Hz, 2H), 2.22 (d, J = 1.2 Hz, 3H), 1.28 (t, J = 7.1 Hz, 3H). |
| Example 50 | 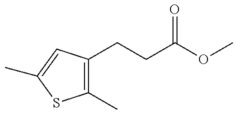 | / | 198.1 | ¹H-NMR (300 MHz, Chloroform-d, ppm) δ 6.47 (d, J = 1.4 Hz, 1H), 3.70 (s, 3H), 2.79 (dd, J = 8.7, 6.8 Hz, 2H), 2.60-2.49 (m, 2H), 2.40 (t, J = 0.9 Hz, 3H), 2.32 (s, 3H). |
| Example 51 | 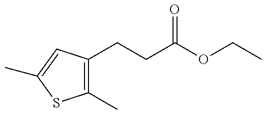 | 213.2 | / | ¹H-NMR (300 MHz, Chloroform-d, ppm) δ 6.48 (d, J = 1.3 Hz, 1H), 4.15 (q, J = 7.1 Hz, 2H), 2.78 (dd, J = 8.8, 6.7 Hz, 2H), 2.59-2.47 (m, 2H), 2.39 (t, J = 0.9 Hz, 3H), 2.32 (s, 3H), 1.27 (t, J = 7.1 Hz, 3H). |
| Example 52 | 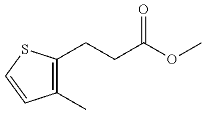 | / | 184.1 | ¹H-NMR (300 MHz, Chloroform-d, ppm) δ 7.06 (d, J = 5.1 Hz, 1H), 6.80 (d, J = 5.1 Hz, 1H), 3.72 (s, 3H), 3.09 (dd, J = 8.9, 6.7 Hz, 2H), 2.65 (dd, J = 8.6, 7.0 Hz, 2H), 2.20 (s, 3H). |
| Example 53 | 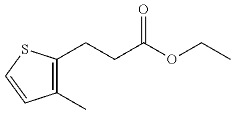 | / | 198.1 | ¹H-NMR (300 MHz, Chloroform-d, ppm) δ 7.05 (d, J = 5.1 Hz, 1H), 6.80 (d, J = 5.1 Hz, 1H), 4.17 (q, J = 7.1 Hz, 2H), 3.18-3.03 (m, 2H), 2.73-2.58 (m, 2H), 2.22 (d, J = 1.1 Hz, 0H), 2.20 (s, 3H), 1.28 (t, J = 7.1 Hz, 3H). |
| Example 54 | 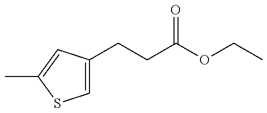 | / | 198.1 | ¹H-NMR (300 MHz, Chloroform-d, ppm) δ 6.70 (q, J = 1.1 Hz, 1H), 6.60 (t, J = 1.3 Hz, 1H), 4.13 (q, J = 7.1 Hz, 2H), 2.87 (dd, J = 8.2, 7.2 Hz, 2H), 2.64-2.53 (m, 2H), 2.44 (d, J = 1.1 Hz, 3H), 1.24 (t, J = 7.1 Hz, 3H). |
| Example 55 | 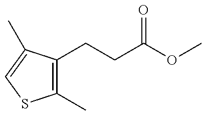 | / | 198.1 | ¹H-NMR (400 MHz, Chloroform-d, ppm) δ 6.69 (d, J = 1.5 Hz, 1H), 3.71 (s, 3H), 2.88-2.80 (m, 2H), 2.51-2.42 (m, 2H), 2.40 (s, 3H), 2.19 (d, J = 1.1 Hz, 3H). |
| Example 56 | 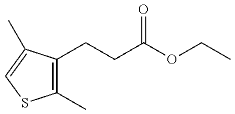 | 213.1 | / | ¹H-NMR (300 MHz, DMSO-d₆, ppm) δ 6.82 (d, J = 1.2 Hz, 1H), 4.05 (q, J = 7.1 Hz, 2H), 2.71 (dd, J = 8.7, 6.9 Hz, 2H), 2.40 (dd, J = 8.7, 7.0 Hz, 2H), 2.31 (s, 3H), 2.11 (d, J = 1.1 Hz, 3H), 1.17 (t, J = 7.1 Hz, 3H). |
| Example 57 | 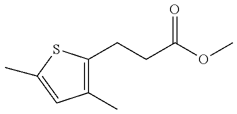 | / | 198.1 | ¹H-NMR (300 MHz, Chloroform-d, ppm) δ 6.45 (d, J = 1.2 Hz, 1H), 3.71 (s, 3H), 3.07-2.95 (m, 2H), 2.61 (dd, J = 8.6, 6.9 Hz, 2H), 2.40 (d, J = 1.1 Hz, 3H), 2.11 (s, 3H). |
| Example 58 | 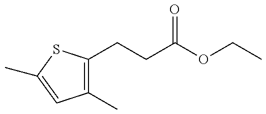 | / | 212.1 | ¹H-NMR (400 MHz, Chloroform-d, ppm) δ 6.45 (d, J = 1.4 Hz, 1H), 4.17 (q, J = 7.1 Hz, 2H), 3.01 (t, J = 7.8 Hz, 2H), 2.64-2.55 (m, 2H), 2.40 (d, J = 1.2 Hz, 3H), 2.11 (s, 3H), 1.28 (t, J = 7.1 Hz, 3H). |
| Example 59 | 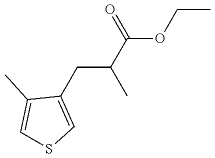 | 213.0 | / | ¹H-NMR (300 MHz, DMSO-d₆, ppm) δ 7.08 (s, 2H), 4.03 (q, J = 7.1 Hz, 2H), 2.77 (ddd, J = 32.2, 13.8, 7.3 Hz, 2H), 2.56 (dd, J = 13.7, 6.5 Hz, 1H), 2.14 (d, J = 0.7 Hz, 3H), 1.27-1.04 (m, 6H). |
| Example 60 | 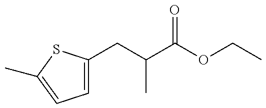 | / | 212.1 | ¹H-NMR (400 MHz, Chloroform-d, ppm) δ 6.61-6.53 (m, 2H), 4.15 (q, J = 7.1 Hz, 2H), 3.15 (dd, J = 14.7, 7.0 Hz, 1H), 2.85 (dd, J = 14.7, 7.2 Hz, 1H), 2.72 (h, J = 7.0 Hz, 1H), 2.44 (d, J = 1.1 Hz, 3H), 1.32-1.15 (m, 6H). |
| Example 61 | 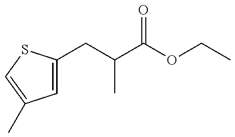 | 213.1 | / | ¹H-NMR (300 MHz, DMSO-d₆, ppm) δ 6.88 (p, J = 1.1 Hz, 1H), 6.66 (d, J = 1.4 Hz, 1H), 4.05 (q, J = 7.1 Hz, 2H), 3.08-2.92 (m, 1H), 2.92-2.76 (m, 1H), 2.68 (h, J = 7.1 Hz, 1H), 2.14 (d, J = 1.1 Hz, 3H), 1.24-1.00 (m, 6H). |
| Example 62 | 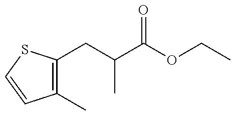 | / | 212.1 | ¹H-NMR (300 MHz, DMSO-d₆, ppm) δ 7.23 (d, J = 5.1 Hz, 1H), 6.81 (d, J = 5.1 Hz, 1H), 4.04 (q, J = 7.1 Hz, 2H), 3.08-2.94 (m, 1H), 2.92-2.76 (m, 1H), 2.74-2.56 (m, 1H), 2.11 (s, 3H), 1.23-1.06 (m, 6H). |

### Example 63

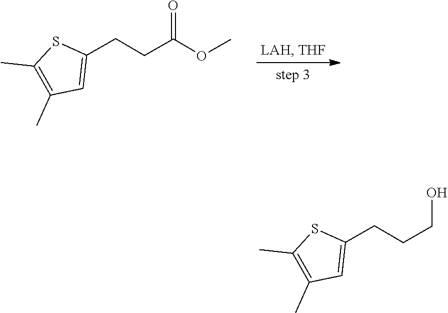

A solution of methyl 3-(4,5-dimethylthiophen-2-yl) propanoate (2 g, 10.09 mmol, 1.00 equiv) and LAH (0.46 g, 12.10 mmol, 1.20 equiv) in THF (20 mL) was stirred at 0° C. for 6 h under air atmosphere. The reaction was quenched with sodium sulfate decahydrate at 0° C. The resulting mixture was filtered, the filter cake was washed with THF (3×20 mL). The filtrate was concentrated under reduced pressure. The residue was purified by silica gel column chromatography, eluted with PE/LA (8:1) to afford 3-(4,5-dimethylthiophen-2-yl) propan-6-ol (610.7 mg, 97.02% purity, 35.67% yield) as a light yellow oil. GC-MS: EI+, m/z, M⁺: 170.1. ¹H NMR (400 MHz, Chloroform-d, ppm) δ 6.50 (s, 1H), 3.73 (td, J=6.3, 1.3 Hz, 2H), 2.83 (t, J=7.5 Hz, 2H), 2.30 (s, 3H), 2.09 (s, 3H), 1.97-1.86 (in, 2H).

The following Examples were synthesized in a similar manner to that described for

| | | LC-MS | GC-MS | |
|---|---|---|---|---|
| | | (ES+, | (EI+, | |
| | | m/z, | m/z, | |
| Number | Structure | [M + H]⁺) | [M]⁺ | ¹H-NMR |
| Example 64 | 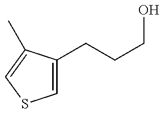 | / | 156.0 | ¹H-NMR (300 MHz, Chloroform-d, ppm) δ 6.98-6.89 (m, 2H), 3.75 (t, J = 6.4 Hz, 2H), 2.70-2.59 (m, 2H), 2.22 (d, J = 0.8 Hz, 3H), 1.92 (d, J = 9.0, 7.6, 6.4 Hz, 2H). |
| Example 65 | 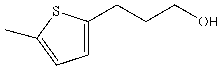 | / | 156.1 | ¹H-NMR (300 MHz, Chloroform-d, ppm) δ 6.64-6.53 (m, 2H), 3.73 (t, J = 6.3 Hz, 2H), 2.88 (t, J = 7.5 Hz, 2H), 2.45 (d, J = 1.1 Hz, 3H), 1.93 (t, J = 7.3, 6.3 Hz, 2H). |
| Example 66 | 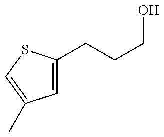 | / | 156.1 | ¹H-NMR (300 MHz, Chloroform-d, ppm) δ 6.71 (p, J = 1.2 Hz, 1H), 6.68- 6.61 (m, 1H), 3.73 (t, J = 6.3 Hz, 2H), 2.96-2.85 (m, 2H), 2.23 (d, J = 1.1 Hz, 3H), 2.03-1.85 (m, 2H), 1.44 (s, 1H). |
| Example 67 | 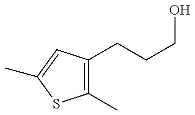 | / | 170.1 | ¹H-NMR (300 MHz, Chloroform-d, ppm) δ 6.49 (d, J = 1.3 Hz, 1H), 3.68 (t, J = 6.4 Hz, 2H), 2.61-2.50 (m, 2H), 2.41 (s, 3H), 2.32 (s, 3H), 1.90-1.75 (m, 2H), 1.43 (s, 2H). |
| Example 68 | 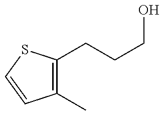 | / | 156.1- | ¹H NMR (300 MHz, Chloroform-d, ppm) δ 7.05 (d, J = 5.1 Hz, 1H), 6.81 (d, J = 5.1 Hz, 1H), 3.73 (t, J = 6.3 Hz, 2H), 2.96-2.81 (m, 2H), 2.19 (s, 3H), 2.00-1.85 (m, 2H), 1.49 (s, 3H). |
| Example 69 | 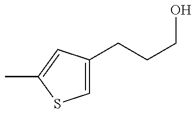 | / | 156.1 | ¹H NMR (300 MHz, DMSO-d₆, ppm) δ 6.83 (q, J = 1.1 Hz, 1H), 6.66 (t, J = 1.3 Hz, 1H), 4.43 (t, J = 5.1 Hz, 1H), 3.40 (td, J = 6.5, 5.1 Hz, 2H), 2.57- 2.46 (m, 1H), 2.40 (d, J = 1.1 Hz, 3H), 1.75-1.60 (m, 2H). |
| Example 70 | 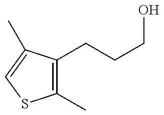 | / | 170.1 | ¹H NMR (400 MHz, Chloroform-d, ppm) δ 6.69 (d, J = 1.2 Hz, 1H), 3.69 (t, J = 6.4 Hz, 2H), 2.64-2.56 (m, 2H), 2.40 (s, 3H), 2.19 (d, J = 1.1 Hz, 3H), 1.81-1.70 (m, 2H), 1.44 (s, 1H). |
| Example 71 | 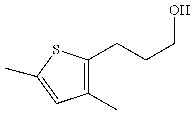 | 171.1 | / | ¹H NMR (400 MHz, Chloroform-d, ppm) δ 6.46 (d, J = 1.5 Hz, 1H), 3.72 (t, J = 6.3 Hz, 2H), 2.78 (t, J = 7.5 Hz, 2H), 2.41 (d, J = 1.1 Hz, 3H), 2.11 (s, 3H), 1.88 (tt, J = 7.4, 6.3 Hz, 2H). |
| Example 72 | 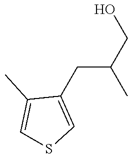 | 171.1 | / | ¹H NMR (300 MHz, DMSO-d₆, ppm) δ 7.10-7.01 (m, 2H), 4.51 (t, J = 5.3 Hz, 1H), 3.37-3.26 (m, 1H), 3.30- 3.18 (m, 1H), 2.63 (dd, J = 14.2, 5.7 Hz, 1H), 2.20 (dd, J = 14.2, 8.4 Hz, 1H), 2.13 (d, J = 0.8 Hz, 3H), 1.76 (dp, J = 8.3, 6.2 Hz, 1H), 0.83 (d, J = 6.7 Hz, 3H). |
| Example 73 | 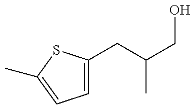 | / | 170.1 | ¹H NMR (400 MHz, Chloroform-d, ppm) δ 6.61-6.54 (m, 2H), 3.62-3.49 (m, 2H), 2.89 (dd, J = 14.7, 6.4 Hz, 1H), 2.64 (dd, J = 14.6, 7.6 Hz, 1H), 2.46 (s, 3H), 2.03-1.87 (m, 1H), 0.99 (d, J = 6.8 Hz, 3H). |
| Example 74 | 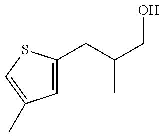 | 171.1 | / | ¹H NMR (300 MHz, DMSO-d₆, ppm) δ 6.86 (t, J = 1.3 Hz, 1H), 6.63 (s, 1H), 4.53 (t, J = 5.2 Hz, 1H), 3.27 (td, J = 5.6, 2.6 Hz, 2H), 2.85 (dd, J = 14.6, 5.7 Hz, 1H), 2.50-2.40 (m, 1H), 2.15 (d, J = 1.1 Hz, 3H), 1.74 (dq, J = 12.9, 6.4 Hz, 1H), 0.83 (d, J = 6.7 Hz, 3H). |
| Example 75 | 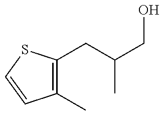 | 171.2 | / | ¹H NMR (300 MHz, DMSO-d₆, ppm) δ 7.20 (d, J = 5.1 Hz, 1H), 6.81 (d, J = 5.1 Hz, 1H), 4.54 (t, J = 5.2 Hz, 1H), 3.37-3.19 (m, 2H), 2.83 (dd, J = 14.6, 5.6 Hz, 1H), 2.43 (dd, J = 14.6, 8.4 Hz, 1H), 2.11 (s, 3H), 1.88-1.65 (m, 1H), 0.83 (d, J = 6.7 Hz, 3H). |

### Example 76

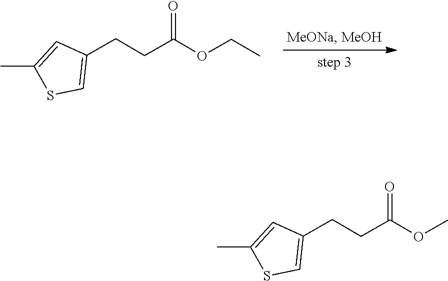

A solution of ethyl 3-(5-methylthiophen-3-yl) propanoate (1.5 g, 7.57 mmol, 1.00 equiv) and MeONa (1.23 g, 22.70 mmol, 3.00 equiv) in MeOH (15 mL) was stirred at room temperature for 3 h under air atmosphere. The resulting mixture was filtered; the filter cake was washed with MeOH (3×5 mL). The filtrate was concentrated under reduced pressure. The residue was purified by silica gel column chromatography, eluted with PE/THF (10:1) to afford methyl 3-(5-methylthiophen-3-yl) propanoate (336 mg, 24.11% yield, 97.16% purity) as a yellow oil. LC-MS: ES+, m/z, [M+H]⁺: 185.1. ¹H NMR (300 MHz, DMSO-d₆, ppm) δ 6.89 (q, J=1.1 Hz, 1H), 6.68 (t, J=1.3 Hz, 1H), 3.59 (s, 3H), 2.76 (ddt, J=8.0, 6.8, 1.1 Hz, 2H), 2.59 (ddd, J=8.2, 7.0, 1.1 Hz, 2H), 2.39 (d, J=1.1 Hz, 3H).

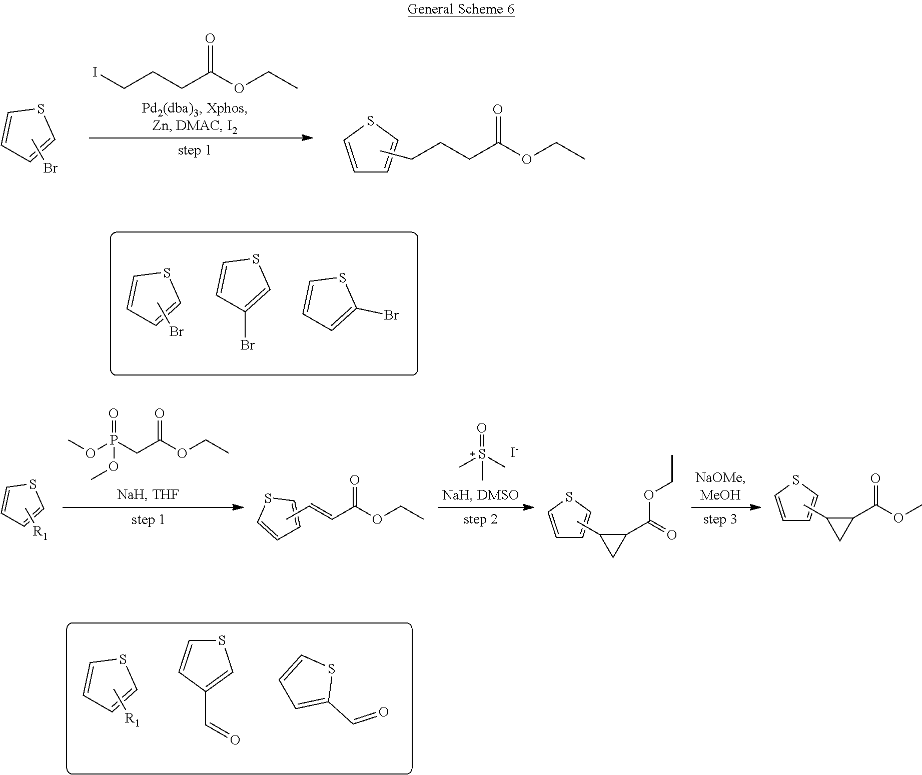

### Example 77

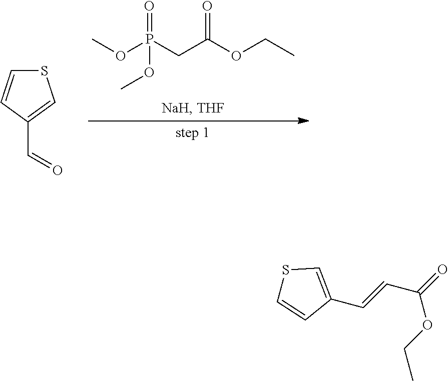

A solution of ethyl 2-(dimethoxyphosphoryl) acetate (8.74 g, 44.58 mmol, 1.00 equiv) in THF (50 mL) was treated with NaH (2.14 g, 89.17 mmol, 2.00 equiv) at 0° C. for 1 h under nitrogen atmosphere followed by the addition of 3-thiophenecarboxaldehyde (5 g, 44.58 mmol, 1.00 equiv) dropwise at 0° C. The resulting mixture was stirred at room temperature for overnight under nitrogen atmosphere. The reaction was quenched with sat. NH₄Cl (aq.) at room temperature. The resulting mixture was extracted with EtOAc (50 mL). The combined organic layers were washed with brine (3×50 mL), dried over anhydrous Na₂SO₄. After filtration, the filtrate was concentrated under reduced pressure. The residue was purified by silica gel column chromatography, eluted with PE/EA (7:1) to afford ethyl (2E)-3-(thiophen-3-yl) prop-2-enoate (5.2 g, 64.00% yield, 97.42% purity) as a yellow oil. GC-MS: (EI+, m/z, M+): 182.0 1H NMR (300 MHz, Chloroform-d, ppm) δ 7.75-7.64 (m, 1H), 7.55-7.48 (m, 1H), 7.41-7.28 (m, 2H), 6.28 (d, J=15.9 Hz, 1H), 4.28 (q, J=7.1 Hz, 2H), 1.35 (t, J=7.1 Hz, 3H).

The following Example was synthesized in a similar manner to that described for Example 77:

| | | GC-MS | |
|---|---|---|---|
| | | (EI+, | |
| Num- | | m/z, | |
| ber | Structure | M⁺) | ¹H-NMR |
| Ex- ample 78 | 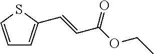 | 182.0 | ¹H NMR (300 MHz, Chloroform-d, ppm) δ 7.86- 7.75 (m, 1H), 7.43-7.35 (m, 1H), 7.31-7.23 (m, 1H), 7.12-7.03 (m, 1H), 6.26 (d, J = 15.7 Hz, 1H), 4.27 (q, J = 7.1 Hz, 2H), 1.35 (t, J = 7.1 Hz, 3H). |

### Example 79

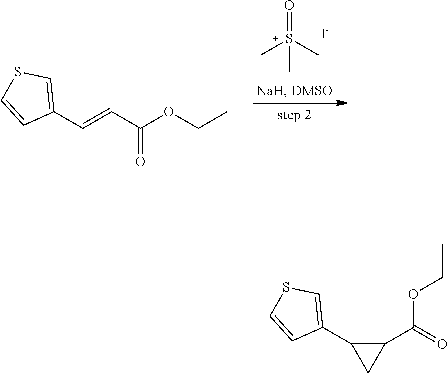

A solution of trimethyl(oxo)-lambda6-sulfanylium iodide (9.06 g, 41.16 mmol, 2.50 equiv) in DMSO (30 mL) was treated with NaH (0.87 g, 36.22 mmol, 2.20 equiv) at 0° C. for 1 h under nitrogen atmosphere followed by the addition of ethyl (2E)-3-(thiophen-3-yl) prop-2-enoate (3 g, 16.46 mmol, 1.00 equiv) dropwise at 0° C. The resulting mixture was stirred at room temperature for overnight under nitrogen atmosphere. Desired product could be detected by GCMS. The reaction was quenched with sat. NH₄Cl (aq.) at room temperature. The resulting mixture was extracted with EtOAc (30 mL). The combined organic layers were washed with water (3×30 mL), dried over anhydrous Na₂SO₄. After filtration, the filtrate was concentrated under reduced pressure. The residue was purified by silica gel column chromatography, eluted with PE/EA (5:1) to afford ethyl 2-(thiophen-3-yl) cyclopropane-1-carboxylate (2.1 g, 65.00% yield, 97.85% purity) as a colorless oil. GC-MS: EI+, m/z, M⁺: 196.1. 1H NMR (300 MHz, Chloroform-d, ppm) δ 7.30-7.23 (m, 1H), 7.03-6.95 (m, 1H), 6.90-6.82 (m, 1H), 4.19 (q, J=7.2 Hz, 2H), 2.63-2.51 (m, 1H), 1.95-1.83 (m, 1H), 1.64-1.52 (m, 1H), 1.36-1.21 (m, 4H).

The following Example was synthesized in a similar manner to that described for Example 79:

| | | GC-MS | |
|---|---|---|---|
| | | (EI+, | |
| Num- | | m/z, | |
| ber | Structure | M⁺) | ¹H-NMR |
| Ex- ample 80 | 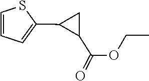 | 196.1 | ¹H NMR (300 MHz, Chloroform-d, ppm) δ 7.12 (dd, J = 5.1, 1.2 Hz, 1H), 6.93 (dd, J = 5.1, 3.5 Hz, 1H), 6.85 (dt, J = 3.5, 1.0 Hz, 1H), 4.20 (q, J = 7.1 Hz, 2H), 2.79-2.66 (m, 1H), 1.95 (ddd, J = 8.5, 5.3, 4.0 Hz, 1H), 1.64 (ddd, J = 9.1, 5.3, 4.5 Hz, 2H), 1.40-1.26 (m, 4H). |

### Example 81

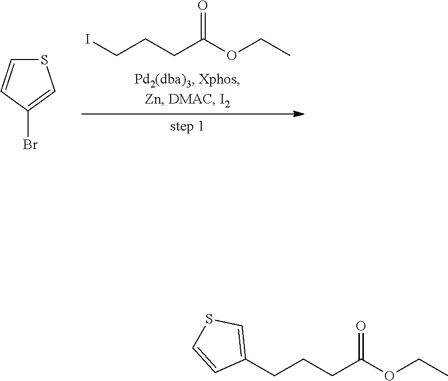

System [A]: A solution of Zn (7.22 g, 110.41 mmol, 3.00 equiv) in DMAC (40 mL) was treated with Iodine (1.40 g, 5.52 mmol, 0.15 equiv) at room temperature for 1 h under nitrogen atmosphere followed by the addition of ethyl 4-iodobutanoate (17.82 g, 73.61 mmol, 2.00 equiv) and Iodine (1.40 g, 5.52 mmol, 0.15 equiv) in DMAC (10 mL) dropwise at room temperature. The resulting mixture was stirred at room temperature for 1 h under nitrogen atmosphere. System [B]: A solution of 3-bromothiophene (6 g, 36.80 mmol, 1.00 equiv), XPhos (3.51 g, 7.36 mmol, 0.20 equiv) and Pd₂(dba)₃ (3.37 g, 3.68 mmol, 0.10 equiv) in DMAC (60 mL) was stirred at room temperature for 1 h under nitrogen atmosphere. To the above System [A] was added System [B] dropwise over 5 min at room temperature. The resulting mixture was stirred at 50° C. for additional 3 h. The reaction was quenched by the addition of sat. NH₄Cl (aq.) (30 mL) at room temperature. The resulting mixture was extracted with EtOAc (60 mL). The combined organic layers were washed with water (3×60 mL), dried over anhydrous Na₂SO₄. After filtration, the filtrate was concentrated under reduced pressure. The residue was purified by silica gel column chromatography, eluted with PE/LA (8:1) to afford ethyl 4-(thiophen-3-yl)butanoate (3.6 g, 49.33% yield, 98.17% purity) as a yellow oil. GC-MS: EI+, m/z, MH: 198.1. 1H NMR (300 MHz, Chloroform-d, ppm) δ7.36-6.92 (m, 3H), 4.15 (q, J=7.2 Hz, 2H), 2.70 (t, J=7.6 Hz, 2H), 2.35 (t, J=7.4 Hz, 2H), 1.98 (p, J=7.5 Hz, 2H), 1.27 (t, J=7.1 Hz, 3H).

The following Example was synthesized in a similar manner to that described for Example 81:

| | | LC-MS | GC-MS | |
|---|---|---|---|---|
| | | (ES+, m/z, | (EI+, m/z, | |
| Number | Structure | [M + H]⁺) | M⁺) | ¹H-NMR |
| Example 82 | 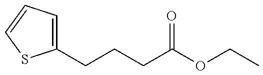 | 199.1 | | ¹H NMR (300 MHz, Chloroform-d, ppm) δ 7.18-7.10 (m, 1H), 6.98-6.89 (m, 1H), 6.85-6.78 (m, 1H), 4.15 (q, J = 7.2 Hz, 2H), 2.90 (t, J = 7.5 Hz, 2H), 2.38 (t, J = 7.4 Hz, 2H), 2.03 (p, J = 7.5 Hz, 2H), 1.28 (td, J = 7.2, 1.1 Hz, 3H). |
| Example 84 | 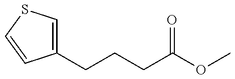 | | 182.0 | ¹H NMR (300 MHz, Chloroform-d, ppm) δ 7.69 (dd, J = 15.9, 0.6 Hz, 1H), 7.55- 7.48 (m, 1H), 7.41-7.28 (m, 2H), 6.28 (d, J = 15.9 Hz, 1H), 4.28 (q, J = 7.1 Hz, 2H), 1.35 (t, J = 7.1 Hz, 3H). |
| Example 86 | 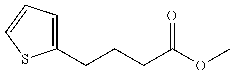 | 185.1 | | ¹H NMR (300 MHz, Chloroform-d, ppm) δ 7.12 (dd, J = 5.2, 1.2 Hz, 1H), 6.92 (dd, J = 5.1, 3.4 Hz, 1H), 6.80 (dt, J = 3.3, 1.2 Hz, 1H), 3.67 (s, 3H), 2.88 (t, J = 7.5 Hz, 2H), 2.38 (t, J = 7.4 Hz, 2H), 2.01 (p, J = 7.5 Hz, 2H). |

### Example 83

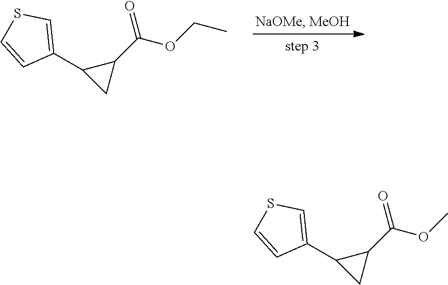

A solution of ethyl 2-(thiophen-3-yl)cyclopropane-1-carboxylate (1.3 g, 6.62 mmol, 1.00 equiv) and MeONa (0.72 g, 13.25 mmol, 2.00 equiv) in MeOH (13 mL) was stirred at room temperature for 2 h under nitrogen atmosphere. Desired product could be detected by GCMS. The resulting mixture was concentrated under vacuum. Column: SunFire prep C18 OBD, 30*150 mm, 5 am; Mobile Phase A: Water (0.1% FA), Mobile Phase B: ACN; Flow rate: 60 mL/min; Gradient: 60% B to 75% B in 8 min; Wave Length: 254/220 nm. This resulted in methyl 2-(thiophen-3-yl)cyclopropane-1-carboxylate (745.1 mg, 61.72% yield, 97.50% purity) as a light yellow oil. GC-MS: EI+, m/z, M⁺: 182.0. 1H NMR (300 MHz, Chloroform-d, ppm) δ 7.12 (dd, J=5.1, 1.2 Hz, 1H), 6.93 (dd, J=5.1, 3.5 Hz, 1H), 6.85 (dt, J=3.5, 1.1 Hz, 1H), 3.75 (s, 3H), 2.80-2.67 (m, 1H), 1.96 (ddd, J=8.5, 5.3, 4.1 Hz, 1H), 1.71-1.59 (m, 1H), 1.42-1.20 (in, 1H).

The following Examples were synthesized in a similar manner to that described for Example 83:

| | | GC-MS | |
|---|---|---|---|
| | | (EI+, | |
| Num- | | m/z, | |
| ber | Structure | M⁺) | ¹H-NMR |
| Ex- am- ple 85 | 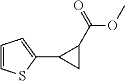 | 182.0 | ¹H NMR (300 MHz, Chloroform-d, ppm) δ 7.12 (dd, J = 5.1, 1.2 Hz, 1H), 6.93 (dd, J = 5.1, 3.5 Hz, 1H), 6.85 (dt, J = 3.5, 1.0 Hz, 1H), 3.75 (s, 3H), 2.73 (ddd, J = 8.7, 6.2, 4.1 Hz, 1H), 1.96 (ddd, J = 8.5, 5.3, 4.1 Hz, 1H), 1.65 (ddd, J = 9.1, 5.4, 4.5 Hz, 1H), 1.48- 1.30 (m, 2H). |

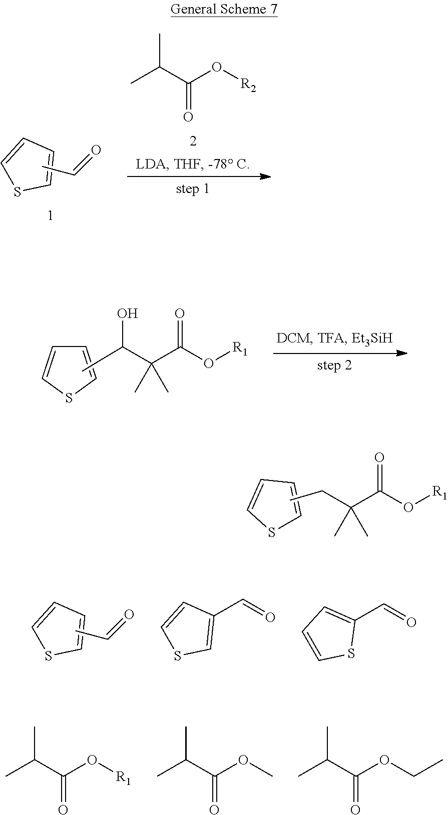

### Example 87

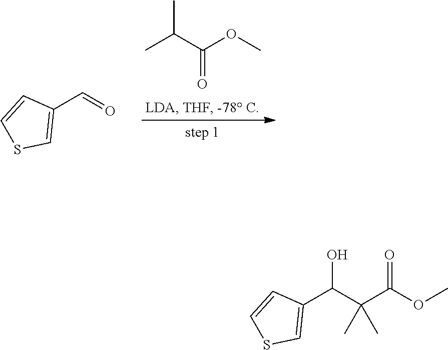

A solution of methyl isobutyrate (4.10 g, 40.16 mmol, 1.50 equiv) in THF (30 mL) was treated with LDA (6.45 g, 60.19 mmol, 2.25 equiv) at −78° C. for 1 h under nitrogen atmosphere followed by the addition of 3-thiophenecarboxaldehyde (3 g, 26.75 mmol, 1.00 equiv) dropwise at−78° C. The reaction was quenched with sat. NH₄Cl (aq.) at room temperature. The resulting mixture was extracted with EtOAc (30 mL). The combined organic layers were washed with water (3×30 mL), dried over anhydrous Na₂SO₄. After filtration, the filtrate was concentrated under reduced pressure. The residue was purified by silica gel column chromatography, eluted with PE/THF (10:1) to afford methyl 3-hydroxy-2,2-dimethyl-3-(thiophen-3-yl) propanoate (5.31 g, 100% purity, 92.4% yield) as a white solid. GC-MS: EI+, m/z, M⁺: 214.0. ¹H-NMR (300 MHz, Chloroform-d, ppm) δ 7.33-7.25 (m, 1H), 7.20 (ddd, J=3.0, 1.3, 0.7 Hz, 1H), 7.05 (dd, J=5.0, 1.3 Hz, 1H), 5.00 (s, 1H), 3.75 (s, 3H), 1.18 (d, J=5.9 Hz, 6H).

The following Example was synthesized in a similar manner to that described for Example 87:

| | | GC-MS | |
|---|---|---|---|
| | | (EI+, | |
| Num- | | m/z, | |
| ber | Structure | M⁺) | ¹H-NMR |
| Ex- am- ple 88 | 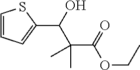 | 228.1 | ¹H-NMR (300 MHz, DMSO- d₆, ppm) δ 3.81 (s, 1H), 2.21 (d, J = 11.8 Hz, 2H), 1.74 (q, J = 9.7, 6.9 Hz, 4H), 1.65- 1.47 (m, 8H), 1.44-1.16 (m, 11H), 0.96-0.76 (m, 3H). |

### Example 89

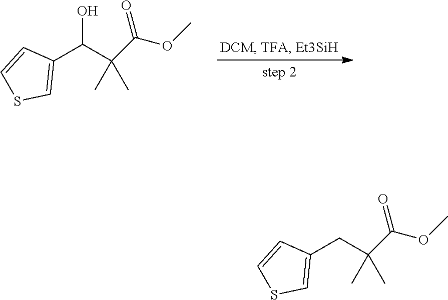

A solution of methyl 3-hydroxy-2,2-dimethyl-3-(thiophen-3-yl) propanoate (5.30 g, 24.73 mmol, 1.00 equiv) in DCM/TFA=10/1 (DCM 50 mL and TFA 5 mL) was treated with triethylsilane (8.63 g, 74.20 mmol, 3.00 equiv) at 70° C. for overnight under nitrogen atmosphere. The mixture was allowed to cool down to room temperature. The mixture was neutralized to pH=7 with saturated NaHCO₃(aq.). The resulting mixture was extracted with EtOAc (30 mL). The combined organic layers were washed with water (3×30 mL), dried over anhydrous Na₂SO₄. After filtration, the filtrate was concentrated under reduced pressure. The residue was purified by silica gel column chromatography, eluted with PE/THF (10:1) to afford methyl 2,2-dimethyl-3-(thiophen-3-yl) propanoate) (2.12 g, 99.87% purity, 42.86% yield) as a colorless oil. GC-MS: (EI+, m/z, M+): 198.1 1H-NMR (300 MHz, Chloroform-d, ppm) δ 7.23 (dd, J=4.9, 3.0 Hz, 1H), 6.95 (ddt, J=2.9, 1.4, 0.7 Hz, 1H), 6.87 (dd, J=4.9, 1.3 Hz, 1H), 3.69 (s, 3H), 2.90 (s, 2H), 1.21 (s, 6H).

The following Examples were synthesized in a similar manner to that described for Example 89:

| | | GC-MS | |
|---|---|---|---|
| | | (EI+, m/z, | |
| Number | Structure | M⁺) | ¹H-NMR |
| Example 90 | 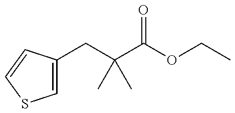 | 212.1 | ¹H-NMR (300 MHz, Chloroform-d, ppm) δ 7.23 (dd, J = 4.9, 2.9 Hz, 1H), 6.96 (d, J = 3.0 Hz, 1H), 6.89 (dd, J = 4.9, 1.3 Hz, 1H), 4.14 (q, J = 7.1 Hz, 2H), 3.52 (s, 0H), 2.90 (s, 2H), 1.26 (t, J = 7.1 Hz, 3H), 1.20 (s, 6H). |
| Example 91 | 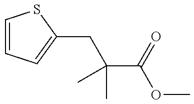 | 198.1 | ¹H-NMR (300 MHz, Chloroform-d, ppm) δ 7.15 (dd, J = 5.2, 1.2 Hz, 1H), 6.94 (dd, J = 5.2, 3.4 Hz, 1H), 6.83- 6.75 (m, 1H), 3.72 (s, 3H), 3.09 (d, J = 0.8 Hz, 2H), 1.25 (s, 6H) |
| Example 92 | 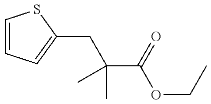 | 212.1 | ¹H-NMR (400 MHz, Chloroform-d, ppm) δ 7.15 (dd, J = 5.2, 1.2 Hz, 1H), 6.94 (dd, J = 5.2, 3.4 Hz, 1H), 6.82- 6.77 (m, 1H), 4.17 (q, J = 7.1 Hz, 2H), 3.09 (s, 2H), 1.29 (t, J = 7.1 Hz, 3H), 1.24 (s, 6H)). |

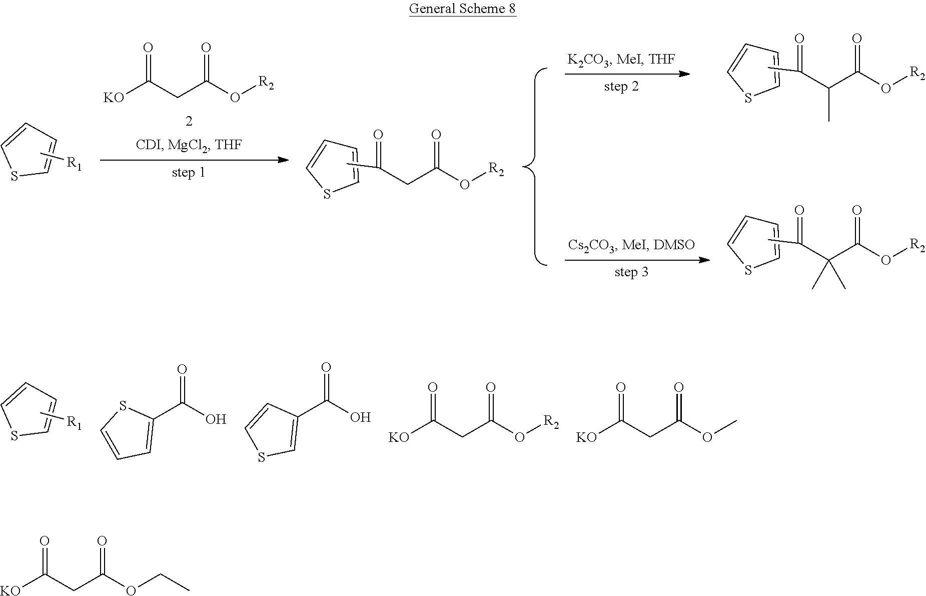

### Example 93

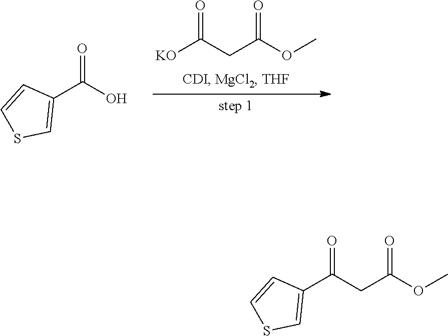

A solution of thiophene-3-carboxylic acid (6 g, 46.82 mmol, 1.00 equiv) and CDI (9.49 g, 58.53 mmol, 1.25 equiv) in THF (60 mL) was stirred at room temperature for 2 h under nitrogen atmosphere. To the above mixture was added MgCl₂ (4.32 g, 45.42 mmol, 0.97 equiv) and 1-methyl 3-potassium propanedioate (10.97 g, 70.23 mmol, 1.50 equiv) in portions over 5 min at room temperature. The resulting mixture was stirred at room temperature for additional 16 h. Desired product could be detected by LCMS. The reaction was quenched with sat. NaHSO₄ (aq.) at room temperature. The resulting mixture was extracted with EtOAc (3×30 mL). The combined organic layers were washed with brine (3×30 mL), dried over anhydrous Na₂SO₄. After filtration, the filtrate was concentrated under reduced pressure. The residue was purified by silica gel column chromatography, eluted with PE/EA (3:1) to afford methyl 3-oxo-3-(thiophen-3-yl)propanoate (6.1 g, 70.73% yield, 99.6% purity) as a colorless oil. LCMS: ES, m/z[M+H]⁺: 185.1. 1H NMR (400 MHz, Chloroform-d, ppm) δ 8.14 (dd, J=3.0, 1.3 Hz, 1H), 7.58 (dd, J=5.1, 1.3 Hz, 1H), 7.40-7.33 (m, 1H), 3.93 (s, 2H), 3.78 (s, 3H).

The following Examples were synthesized in a similar manner to that described for Example 91:

| | | LC-MS | |
|---|---|---|---|
| | | (ES, m/z, | |
| Number | Structure | [M + H] ⁺) | ¹H-NMR |
| Example 94 | 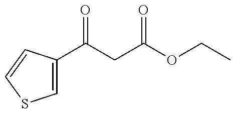 | 199.0 | 1H NMR (400 MHz, Chloroform-d, ppm) δ 8.14 (dd, J = 2.9, 1.3 Hz, 1H), 7.58 (dd, J = 5.1, 1.3 Hz, 1H), 7.40-7.33 (m, 1H), 4.24 (q, J = 7.1 Hz, 2H), 3.91 (s, 2H), 1.29 (t, J = 7.1 Hz, 3H). |
| Example 95 | 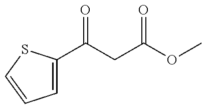 | 185.0 | 1H NMR (400 MHz, Chloroform-d, ppm) δ 7.77 (dd, J = 3.8, 1.1 Hz, 1H), 7.73 (dd, J = 5.0, 1.1 Hz, 1H), 7.18 (dd, J = 5.0, 3.8 Hz, 1H), 3.96 (s, 2H), 3.78 (s, 3H). |
| Example 96 | 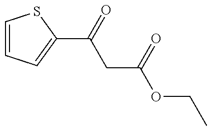 | 199.1 | 1H NMR (400 MHz, Chloroform-d, ppm) δ 7.77 (dd, J = 3.8, 1.1 Hz, 1H), 7.72 (dd, J = 5.0, 1.1 Hz, 1H), 7.17 (dd, J = 4.9, 3.8 Hz, 1H), 4.24 (q, J = 7.1 Hz, 2H), 3.94 (s, 2H), 1.29 (t, J = 7.1 Hz, 3H). |

### Example 97

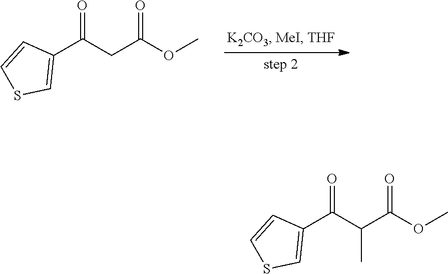

A solution of methyl 3-oxo-3-(thiophen-3-yl) propanoate (2 g, 10.86 mmol, 1.00 equiv) in THF (20 mL) was treated with K₂CO₃ (3.75 g, 27.14 mmol, 2.50 equiv) at room temperature for 5 min under nitrogen atmosphere followed by the addition of Mel (4.62 g, 32.57 mmol, 3.00 equiv) dropwise at 0° C. The resulting mixture was stirred at 60° C. for 1 h under nitrogen atmosphere. Desired product could be detected by LCMS. The reaction was quenched with sat. NH₄Cl (aq.) at 0° C. The resulting mixture was extracted with EtOAc (3×10 mL). The combined organic layers were washed with water (3×10 mL), dried over anhydrous Na₂SO₄. After filtration, the filtrate was concentrated under reduced pressure. The residue was purified by silica gel column chromatography, eluted with PE/LA (3:1) to afford methyl 2-methyl-3-oxo-3-(thiophen-3-yl)propanoate (603.7 mg, 28.05% yield, 98.74% purity) as a colorless oil. LCMS: ES, m/z, [M+H]+: 199.1 1H NMR (300 MHz, Methanol-d4, ppm) δ 8.43 (dd, J=2.8, 1.3 Hz, 1H), 7.60-7.55 (m, 1H), 7.54-7.49 (m, 1H), 4.48 (q, J=7.1 Hz, 1H), 3.70 (s, 3H), 1.43 (d, J=7.1 Hz, 3H).

The following Examples were synthesized in a similar manner to that described for Example 97:

| | | LC-MS | |
|---|---|---|---|
| | | (ES, m/z, | |
| Number | Structure | [M + H] ⁺) | ¹H-NMR |
| Example 98 | 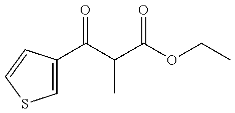 | 213.0 | 1H NMR (300 MHz, Chloroform-d, ppm) δ 8.17 (dd, J = 2.9, 1.3 Hz, 1H), 7.60 (dd, J = 5.1, 1.3 Hz, 1H), 7.35 (dd, J = 5.1, 2.9 Hz, 1H), 4.27-4.12 (m, 3H), 1.51 (d, J = 7.1 Hz, 3H), 1.21 (t, J = 7.1 Hz, 3H). |
| Example 99 | 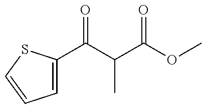 | 199.1 | 1H NMR (300 MHz, Chloroform-d, ppm) δ 7.81 (dd, J = 3.9, 1.1 Hz, 1H), 7.72 (dd, J = 5.0, 1.1 Hz, 1H), 7.18 (dd, J = 5.0, 3.8 Hz, 1H), 4.28 (q, J = 7.1 Hz, 1H), 3.73 (s, 3H), 1.54 (d, J = 7.1 Hz, 3H). |
| Example 100 | 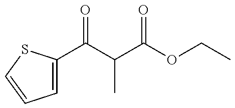 | 213.1 | 1H NMR (300 MHz, Chloroform-d, ppm) δ 7.81 (dd, J = 3.8, 1.1 Hz, 1H), 7.71 (dd, J = 5.0, 1.1 Hz, 1H), 7.17 (dd, J = 5.0, 3.8 Hz, 1H), 4.31-4.12 (m, 3H), 1.53 (d, J = 7.1 Hz, 3H), 1.23 (t, J = 7.1 Hz, 3H). |

### Example 101

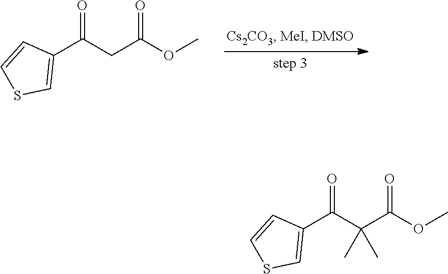

A solution of methyl 3-oxo-3-(thiophen-3-yl)propanoate (2 g, 10.86 mmol, 1.00 equiv) in DMSO (20 mL) was treated with Cs₂CO₃ (7.78 g, 23.89 mmol, 2.20 equiv) at room temperature for 5 min under nitrogen atmosphere followed by the addition of Mel (4.61 g, 32.57 mmol, 3.00 equiv) dropwise at 0° C. The resulting mixture was stirred at room temperature for 16 h under nitrogen atmosphere. Desired product could be detected by LCMS. The reaction was quenched with water at 0° C. The resulting mixture was extracted with EtOAc (3×10 mL). The combined organic layers were washed with water (3×10 mL), dried over anhydrous Na₂SO₄. After filtration, the filtrate was concentrated under reduced pressure. The residue was purified by silica gel column chromatography, eluted with PE/LA (3:1) to afford methyl 2,2-dimethyl-3-oxo-3-(thiophen-3-yl) propanoate (622.7 mg, 27.02% yield, 98.86% purity) as a yellow oil. LCMS: ES, m/z, [M+H]⁺: 213.1. 1H NMR (300 MHz, Chloroform-d, ppm) δ 8.03 (dd, J=2.9, 1.3 Hz, 1H), 7.50 (dd, J=5.1, 1.3 Hz, 1H), 7.31 (dd, J=5.2, 2.9 Hz, 1H), 3.68 (s, 3H), 1.55 (s, 6H).

The following Examples were synthesized in a similar manner to that described for Example 101:

| | | LC-MS | |
|---|---|---|---|
| | | (ES, m/z, | |
| Number | Structure | [M + H] ⁺) | ¹H-NMR |
| Example 102 | 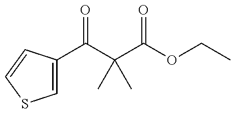 | 227.1 | 1H NMR (300 MHz, Chloroform-d, ppm) δ 8.03 (dd, J = 2.9, 1.3 Hz, 1H), 7.51 (dd, J = 5.1, 1.3 Hz, 1H), 7.30 (dd, J = 5.2, 2.9 Hz, 1H), 4.15 (q, J = 7.2 Hz, 2H), 1.55 (s, 6H), 1.11 (t, J = 7.2 Hz, 3H). |
| Example 103 | 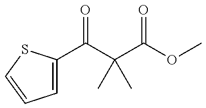 | 213.1 | 1H NMR (300 MHz, Chloroform-d, ppm) δ 7.63 (dd, J = 5.0, 1.1 Hz, 1H), 7.58 (dd, J = 3.9, 1.0 Hz, 1H), 7.11 (dd, J = 5.0, 3.9 Hz, 1H), 3.69 (s, 3H), 1.58 (s, 6H). |
| Example 104 | 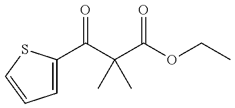 | 227.1 | 1H NMR (300 MHz, Methanol-d4, ppm) δ 8.43 (dd, J = 2.8, 1.3 Hz, 1H), 7.60-7.55 (m, 1H), 7.54-7.49 (m, 1H), 4.48 (q, J = 7.1 Hz, 1H), 3.70 (s, 3H), 1.43 (d, J = 7.1 Hz, 3H). |

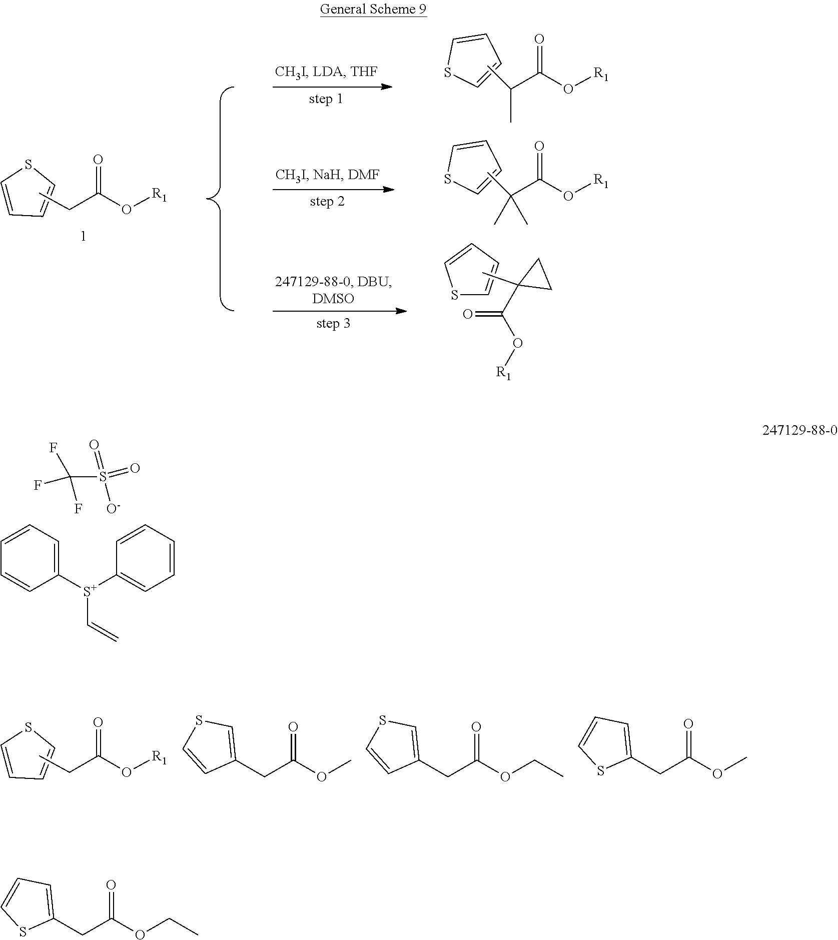

### Example 105

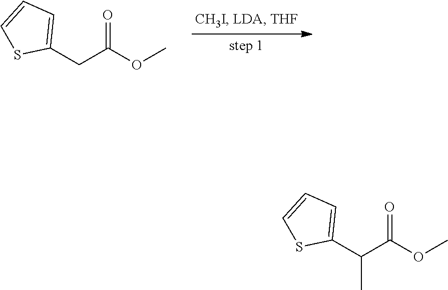

A solution of methyl 2-(thiophen-2-yl)acetate (2 g, 12.80 mmol, 1.00 equiv) in THF (20 mL) was treated with LDA (1.44 g, 13.44 mmol, 1.05 equiv) at −78° C. for 1 h under nitrogen atmosphere followed by the addition of CH₃I (3.63 g, 25.61 mmol, 2.00 equiv) dropwise at −78° C. The resulting mixture was stirred at −78° C. to temperature for 2 h under nitrogen atmosphere. Desired product could be detected by GCMS. The reaction was quenched with sat. NH₄Cl (aq.) at room temperature. The resulting mixture was extracted with EtOAc (30 mL). The combined organic layers were washed with water (3×30 mL), dried over anhydrous Na₂SO₄. After filtration, the filtrate was concentrated under reduced pressure. The residue was purified by silica gel column chromatography, eluted with PE/EA (10:1) to afford methyl 2-(thiophen-2-yl)propanoate (313.4 mg, 14.38% yield, 100% purity) as a yellow oil. GC-MS: EI+, m/z, M⁺: 170.0000. 1H NMR (300 MHz, Chloroform-d, ppm) δ 7.27-7.18 (m, 1H), 7.02-6.94 (m, 2H), 4.04 (q, J=7.2 Hz, 1H), 3.73 (s, 3H), 1.61 (d, J=7.2 Hz, 3H).

The following Examples were synthesized in a similar manner to that described for Example 105:

| | | GC-MS | |
|---|---|---|---|
| | | (EI+, m/z, | |
| Number | Structure | M⁺) | ¹H-NMR |
| Example 106 | 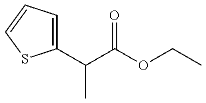 | 184.1000 | 1H NMR (300 MHz, Chloroform-d, ppm) δ 7.28-7.18 (m, 1H), 7.02-6.92 (m, 2H), 4.19 (qd, J = 7.1, 0.9 Hz, 2H), 4.02 (q, J = 7.1 Hz, 1H), 1.60 (d, J = 7.2 Hz, 3H), 1.28 (t, J = 7.1 Hz, 3H). |
| Example 107 | 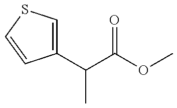 | 170.0 | 1H NMR (300 MHz, Chloroform-d, ppm) δ 7.30 (dd, J = 5.0, 2.9 Hz, 1H), 7.19-7.12 (m, 1H), 7.12-7.04 (m, 1H), 3.87 (q, J = 7.2 Hz, 1H), 3.71 (s, 3H), 1.54 (d, J = 7.2 Hz, 3H). |
| Example 108 | 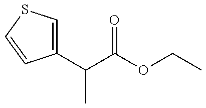 | 184.1 | 1H NMR (300 MHz, Chloroform-d, ppm) δ 7.34-7.25 (m, 1H), 7.17-7.13 (m, 1H), 7.11-7.06 (m, 1H), 4.27-4.06 (m, 2H), 3.85 (q, J = 7.2 Hz, 1H), 1.57- 1.49 (m, 3H), 1.26 (t, J = 7.1 Hz, 3H). |

### Example 109

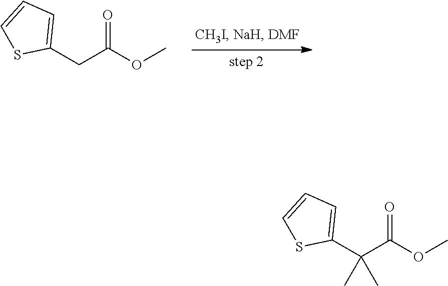

A solution of methyl 2-(thiophen-2-yl)acetate (2 g, 12.80 mmol, 1.00 equiv) in DMF (20 mL) was treated with NaH (0.92 g, 38.41 mmol, 3.00 equiv) and 18-crown-6 (1.69 g, 6.40 mmol, 0.50 equiv) at 0° C. for 1 h under nitrogen atmosphere followed by the addition of CH₃I (5.45 g, 38.41 mmol, 3.00 equiv) dropwise at 0° C. The resulting mixture was stirred at room temperature for 2 h under nitrogen atmosphere. Desired product could be detected by GCMS. The reaction was quenched with sat. NH₄Cl (aq.) at room temperature. The resulting mixture was extracted with EtOAc (30 mL). The combined organic layers were washed with water (3×30 mL), dried over anhydrous Na₂SO₄. After filtration, the filtrate was concentrated under reduced pressure. The residue was purified by silica gel column chromatography, eluted with PE/EA (10:1) to afford methyl 2-methyl-2-(thiophen-2-yl)propanoate (706.2 mg, 29.93% yield, 99.55% purity) as a yellow oil. GC-MS: EI+, m/z, M⁺: 184.0000. 1H NMR (300 MHz, Chloroform-d, ppm) δ 7.28-7.18 (m, 1H), 7.01-6.92 (m, 2H), 3.71 (s, 3H), 1.69 (s, 6H).

The following Examples were synthesized in a similar manner to that described for Example 109:

| | | LC-MS | GC-MS | |
|---|---|---|---|---|
| | | (ES, m/z, | (EI+, m/z, | |
| Number | Structure | [M + H] ⁺) | M⁺) | ¹H-NMR |
| Example 110 | 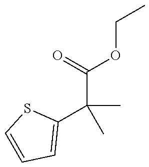 | 199.05 | / | 1H NMR (300 MHz, Chloroform- d, ppm) δ 7.21 (dd, J = 4.6, 1.8 Hz, 1H), 7.02-6.92 (m, 2H), 4.16 (q, J = 7.1 Hz, 2H), 1.68 (s, 6H), 1.25 (t, J = 7.1 Hz, 3H). |
| Example 111 | 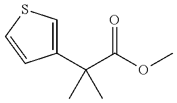 | / | 184.1000 | 1H NMR (300 MHz, Chloroform- d, ppm) δ 7.33-7.25 (m, 1H), 7.15-7.12 (m, 1H), 7.11-7.08 (m, 1H), 3.68 (s, 3H), 1.61 (s, 6H). |
| Example 112 | 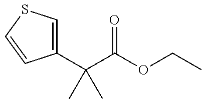 | / | 198.1000 | 1H NMR (300 MHz, Chloroform- d, ppm) δ 7.33-7.24 (m, 1H), 7.14-7.12 (m, 1H), 7.12-7.09 (m, 1H), 4.15 (q, J = 7.1 Hz, 2H), 1.60 (s, 6H), 1.23 (t, J = 7.1 Hz, 3H). |

### Example 113

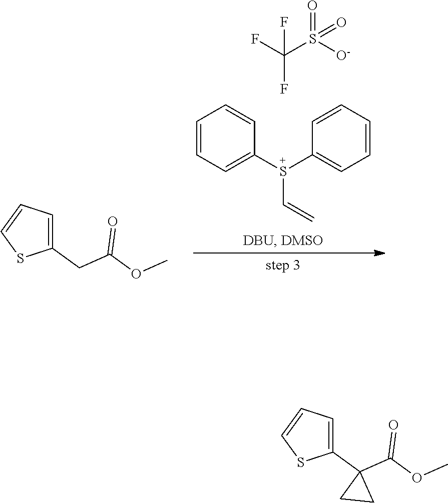

A solution of methyl 2-(thiophen-2-yl)acetate (2 g, 12.80 mmol, 1.00 equiv) in DMSO (20 mL) was treated with ethenyldiphenylsulfanium trifluoromethanesulfonate (5.57 g, 15.37 mmol, 1.20 equiv) at room temperature for 5 min under nitrogen atmosphere followed by the addition of DBU (5.85 g, 38.41 mmol, 3.00 equiv) dropwise at 0° C. The resulting mixture was stirred at room temperature for 2 h under nitrogen atmosphere. Desired product could be detected by GCMS. The reaction was quenched with water at room temperature. The resulting mixture was extracted with EtOAc (30 mL). The combined organic layers were washed with water (3×30 mL), dried over anhydrous Na₂SO₄. After filtration, the filtrate was concentrated under reduced pressure. The residue was purified by silica gel column chromatography, eluted with PE/LA (10:1) to afford methyl 1-(thiophen-2-yl)cyclopropane-1-carboxylate (684.9 mg, 29.35% yield, 100% purity) as a light yellow oil. GC-MS: EI+, m/z, M⁺: 182.0. 1H NMR (300 MHz, Chloroform-d, ppm) δ 7.23 (dd, J=4.9, 1.6 Hz, 1H), 7.00-6.90 (m, 2H), 3.70 (s, 3H), 1.72 (q, J=4.0 Hz, 2H), 1.35 (q, J=4.0 Hz, 2H).

The following Examples were synthesized in a similar manner to that described for Example 113:

| | | GC-MS | |
|---|---|---|---|
| | | (EI+, m/z, | |
| Number | Structure | M⁺) | ¹H-NMR |
| Example 114 | 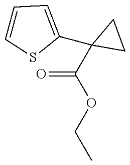 | 196.1 | 1H NMR (300 MHz, Chloroform-d, ppm) δ 7.22 (dd, J = 4.6, 1.9 Hz, 1H), 6.98-6.89 (m, 2H), 4.17 (q, J = 7.1 Hz, 2H), 1.72 (q, J = 4.0 Hz, 2H), 1.34 (q, J = 4.0 Hz, 2H), 1.24 (t, J = 7.1 Hz, 3H). |
| Example 115 | 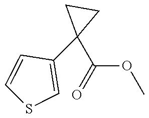 | 182.1 | 1H NMR (300 MHz, Chloroform-d, ppm) δ 7.31-7.23 (m, 1H), 7.18-7.13 (m, 1H), 7.12-7.07 (m, 1H), 3.69 (s, 3H), 1.61 (q, J = 3.9 Hz, 2H), 1.21 (q, J = 3.9 Hz, 2H). |
| Example 116 | 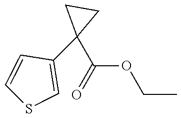 | 196.1000 | 1H NMR (300 MHz, Chloroform-d, ppm) δ 7.30-7.22 (m, 1H), 7.17-7.13 (m, 1H), 7.12-7.06 (m, 1H), 4.15 (q, J = 7.1 Hz, 2H), 1.60 (q, J = 3.9 Hz, 2H), 1.27-1.22 (m, 2H), 1.22-1.17 (m, 3H). |

### Example 128: Odor Profile Determination

Odor profile for selected compounds within the scope of the disclosure is determined using assessments by one Master Perfumer and 4-6 trained sensory panelists. Sensory panelists are trained on our internal taxonomy with reference materials that include 11 grand families, and 62 subfamilies. Panelists are experienced in, but not trained in, an additional several hundred odor descriptors. Panelists are trained on usage of a rating scale, which also includes internal references. Panelists are not advanced to compound evaluation until they have completed and received a passing score on a Final Exam on the taxonomy that was curated by our perfumery team.

A single score is generated for the sensory panel by taking the arithmetic mean of all panelist scores for a given sample, for a given attribute. Alternatively, a single score may be generated by fitting all panelist scores to a model that can correct for differences in the intercept or slope of the latent function through which each panelist produces a rating given a percept, i.e. correction for inter-panelist variation.

The Master Perfumer, possessing a deep knowledge of fragrance ingredients, high level technical expertise, and who is recognized for their experience in creating complex perfumes, reviews, may augment sensory panel scores for improved accuracy.

Test samples are dissolved at a concentration of 10% w/v in ethanol. At time 0 (t=0), new test blotters (White Paper Paddle Shaped Perfumery Blotters, measuring 5×0.5 inches) are dipped into the 10% solution of the text compound. Odor descriptions at time t=0 are captured within 1-2 minutes of wetting the blotter to allow for evaporation of most of the ethanol solvent, permitting a more accurate determination of the test compound's odor. Assessments are made at ambient temperature in a benchtop laboratory setting. The procedure is repeated at 2 hours and at 24 hours.

The results are shown in the table below:

Intensity: 1-10 (max) at t = 0, 10% EtOH solution on blotter paper
+ = 0-3; ++ = 4-7; +++ = 8-10

| | | | Intensity | Intensity | Intensity |
|---|---|---|---|---|---|
| Ex. | | Primary | (1-10) at | (1-10) at | (1-10) at |
| No. | Structure | Notes | 0 hours | 2 hours | 24 hours |
| 8 | 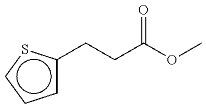 | floral, wet, aldehydic, marine | ++ | + | + |
| 9 | 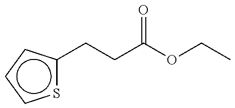 | strawberry, floral, jasmine, juicy | +++ | ++ | + |
| 12 | 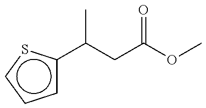 | fruity, watery, green | ++ | ++ | + |
| 13 | 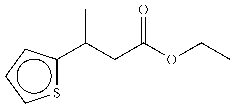 | floral, fresh strawberry | ++ | ++ | + |
| 17 | 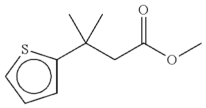 | animalic, green, metallic, leathery | ++ | ++ | ++ |
| 16 | 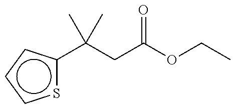 | woody, floral, green, mineral | ++ | ++ | + |
| 15 | 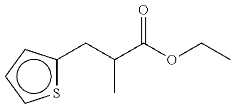 | floral, lactonic, jasmine, peach, plum | ++ | ++ | + |
| 91 | 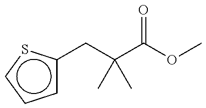 | fruity, woody, creamy | ++ | ++ | + |
| 92 | 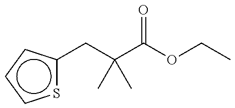 | fruity, berry, herbal, creamy, minty, woody | ++ | + | + |
| 117 | 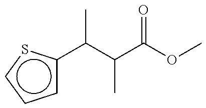 | herbal, rosy, earthy, | ++ | ++ | + |
| 118 | 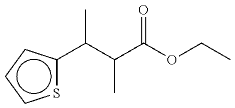 | violet, orris, rosy | ++ | ++ | ++ |
| 119 | 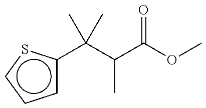 | cedarwood, herbal, musky, rosy | ++ | ++ | + |
| 85 | 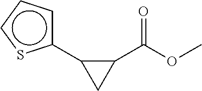 | green, muguet, wet, earthy, woody | ++ | ++ | + |
| 80 | 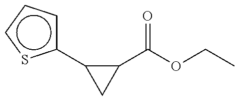 | sulfury, green, mushroom, jasmin | ++ | ++ | ++ |
| 45 | 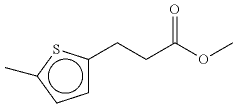 | floral, green, mushroom, rhubarb | ++ | + | + |
| 46 | 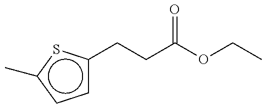 | floral, woody, fruity | ++ | + | + |
| 52 | 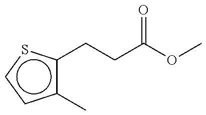 | herbal, apple, pear, jasmine, mushroom | ++ | ++ | + |
| 53 | 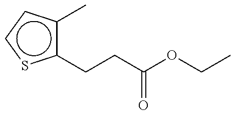 | floral, wet, green, fruity | ++ | ++ | + |
| 48 | 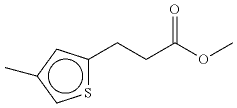 | floral, aldehydic, jasmine | ++ | + | + |
| 49 | 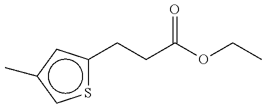 | green, fruity, herbal | ++ | ++ | ++ |
| 60 | 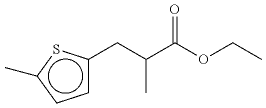 | fruity, lactonic, white floral, apple, banana | ++ | ++ | + |
| 61 | 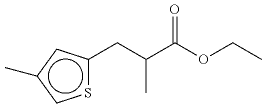 | TBD | | | |
| 62 | 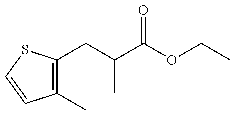 | TBD | | | |
| 42 | 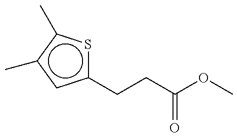 | sweet, fruity, medicinal | + | + | + |
| 47 | 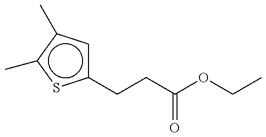 | weak | + | + | + |
| 57 | 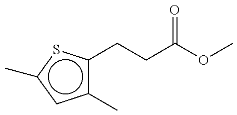 | vanilla, floral, green | ++ | + | + |
| 58 | 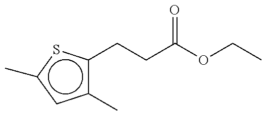 | fruity, apple, pear, white floral, jasmine, mushroom | ++ | + | + |
| 6 | 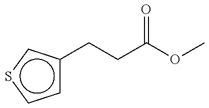 | jasmine, spicy, fruity, strawberry, apple | ++ | ++ | + |
| 7 | 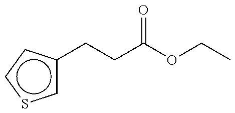 | floral, white floral, jasmine, marine | ++ | + | + |
| 10 | 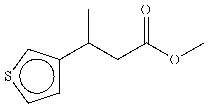 | green, woody, celery, floral, woody | ++ | + | + |
| 11 | 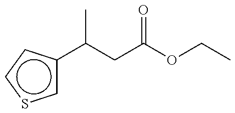 | fruity, floral, herbal, minty | ++ | ++ | + |
| 14 | 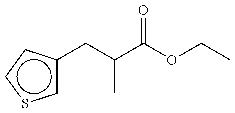 | floral, violet, orris, starchy | ++ | ++ | + |
| 89 | 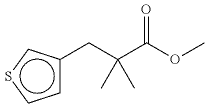 | floral, powdery, fruity, berry, lactonic, creamy | +++ | ++ | + |
| 90 | 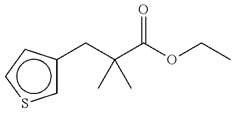 | fruity, berry, rosy, celery, powdery, tea | ++ | ++ | + |
| 120 | 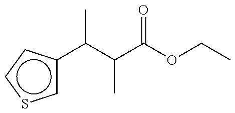 | woody, fruity, coumarinic | ++ | ++ | + |
| 83 | 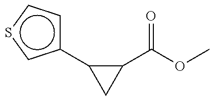 | fruity, grassy, wet | ++ | ++ | + |
| 79 | 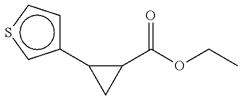 | soulful, sulfury | ++ | ++ | + |
| 54 | 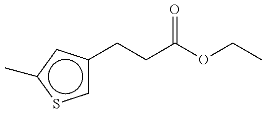 | floral, green, jasmine, wet | ++ | ++ | + |
| 43 | 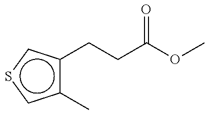 | floral, jasmine, mushroom, watery, metallic | ++ | ++ | + |
| 44 | 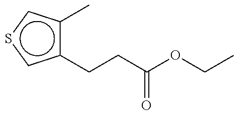 | floral, jasmine, watery | ++ | ++ | ++ |
| 59 | 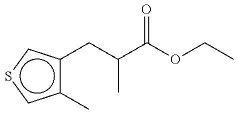 | fruity | ++ | + | + |
| 50 | 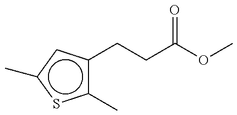 | green, woody, celery, lavender | ++ | + | + |
| 51 | 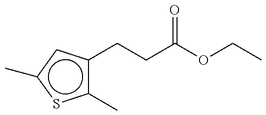 | mineral, marine, fruity, fresh | + | ++ | ++ |
| 55 | 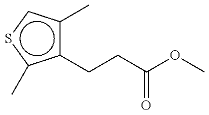 | terpenic, weak, gets stronger over time | ++ | + | + |
| 56 | 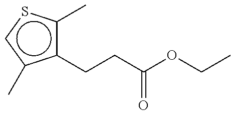 | soft, powdery, floral, nutty | ++ | + | + |
| 76 | 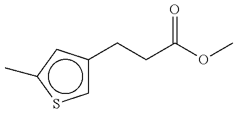 | white floral, green, spicy | ++ | ++ | + |
| 78 | 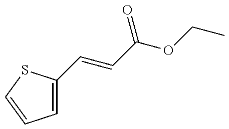 | labdanum, leathery, dried fruit, rosy | ++ | ++ | ++ |
| 3 | 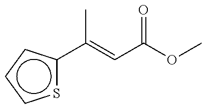 | toasted, green, hyacinth | ++ | ++ | ++ |
| 121 | 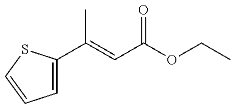 | sweet balsamic, lactonic, driftwood, milky, roasted | ++ | ++ | ++ |
| 5 | 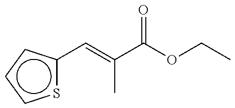 | white floral, strawberry, jasmine | ++ | ++ | ++ |
| 34 | 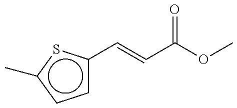 | weak | + | + | + |
| 122 | 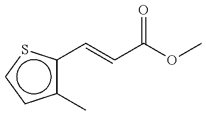 | rosy, powdery, delicate | ++ | ++ | ++ |
| 31 | 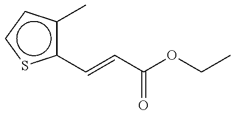 | fruity, floral, rosy, gourmand | ++ | ++ | + |
| 35 | 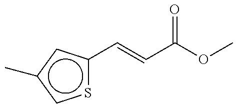 | green, cherry, almond, grapefruit, rhubarb | ++ | ++ | ++ |
| 39 | 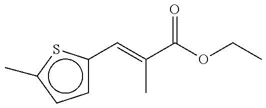 | herbal, animalic, wet, basil | ++ | + | + |
| 41 | 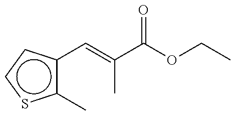 | fruity, apple, pear, wet, green, tropical | ++ | + | + |
| 40 | 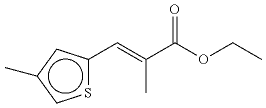 | fruity, apple, pear, peach | + | + | + |
| 32 | 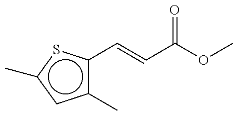 | vanilla, floral, citrus | ++ | + | + |
| 27 | 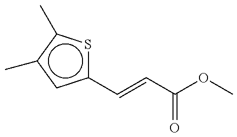 | sweet, caramel, oily | ++ | + | + |
| 28 | 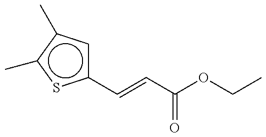 | green | + | + | + |
| 1 | 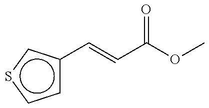 | TBD | | | |
| 77 | 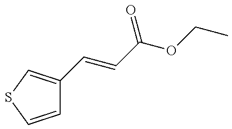 | floral, green, leathery | ++ | ++ | + |
| 2 | 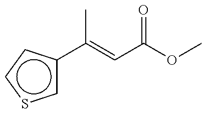 | violet, orange flower, medicinal | ++ | ++ | ++ |
| 4 | 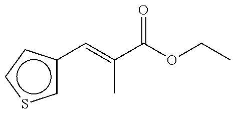 | TBD | | | |
| 123 | 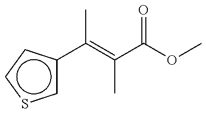 | herbal, leathery, cedarwood | ++ | ++ | + |
| 124 | 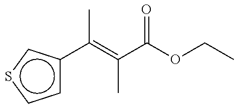 | woody, leather, chemical | ++ | + | + |
| 26 | 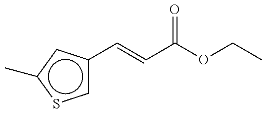 | green, floral | + | + | + |
| 33 | 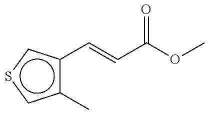 | floral, rosy, powdery | + | + | + |
| 38 | 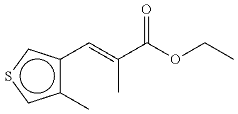 | fruity, apple, pear, citrus, marine | ++ | + | + |
| 29 | 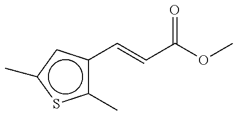 | sulfury, cabbage | ++ | ++ | ++ |
| 30 | 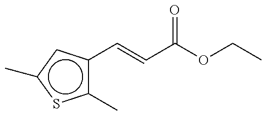 | floral, wet, white floral, banana, grassy, jasmine | ++ | + | ++ |
| 36 | 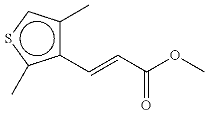 | floral, citrus, rosy | ++ | + | + |
| 37 | 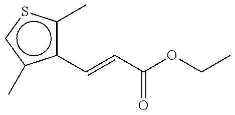 | animalic, sulfury, green | + | + | + |
| 86 | 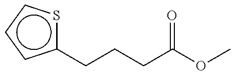 | green | ++ | ++ | ++ |
| 82 | 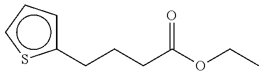 | jasmine, mushroom | + | + | + |
| 84 | 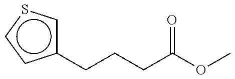 | fruity, white floral, grassy | + | + | + |
| 81 | 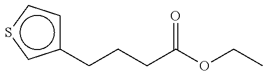 | jasmine, mushroom | + | + | + |
| 106 | 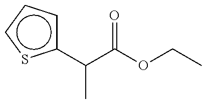 | fruity, mandarin, cassis, green stemmy | ++ | ++ | ++ |
| 110 | 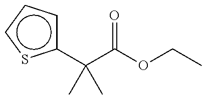 | herbal, minty, fruity, woody | ++ | + | + |
| 113 | 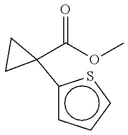 | plum, dark fruit | ++ | + | + |
| 114 | 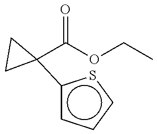 | green, muguet, grapefruit | ++ | ++ | ++ |
| 105 | 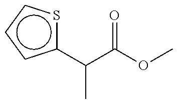 | fruity, grapefruit, peachy, cassis | ++ | + | + |
| 109 | 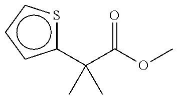 | herbal, minty, citrus, medicinal | ++ | ++ | + |
| 111 | 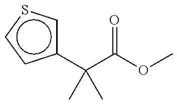 | fruity, leathery, driftwood, dried fruit | + | + | + |
| 108 | 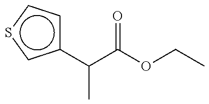 | fruity, violet, orris, strawberry | ++ | ++ | ++ |
| 112 | 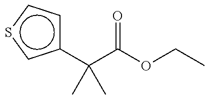 | fruity, apple, pear, cedarwood, creamy | ++ | + | + |
| 115 | 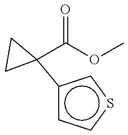 | green, resinous, piney | ++ | ++ | + |
| 116 | 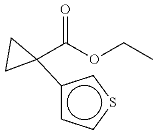 | green, coumarinic, hay, tobacco, rhubarb | ++ | + | + |
| 107 | 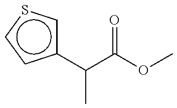 | fruity, woody, leathery | ++ | ++ | + |
| 22 | 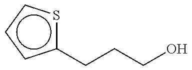 | sweet balsamic, floral, baked pastry | ++ | ++ | + |
| 25 | 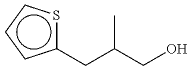 | green, fruity, rhubarb, hyacinth | ++ | ++ | ++ |
| 63 | 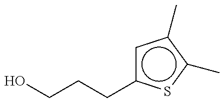 | powdery, muguet | + | + | + |
| 23 | 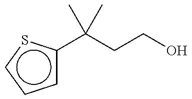 | floral, dry, weak | + | + | + |
| 71 | 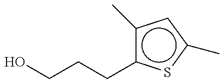 | weak | + | + | + |
| 73 | 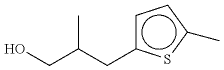 | white grapefruit | + | ++ | + |
| 74 | 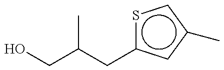 | weak | + | + | + |
| 75 | 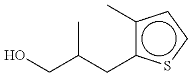 | weak | + | + | + |
| 125 | 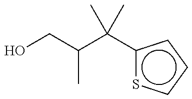 | green, fig, tomato leaf | ++ | ++ | ++ |
| 126 | 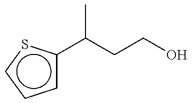 | green, floral | + | + | + |
| 68 | 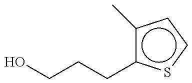 | weak | + | + | + |
| 20 | 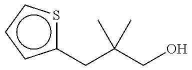 | green, woody, artichoke | ++ | + | + |
| 65 | 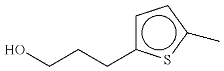 | woody, driftwood, floral | + | + | + |
| 66 | 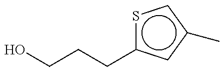 | sweet, caramel, fruity, balsamic | + | + | + |
| 21 | 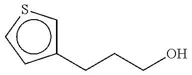 | soulful, floral, soapy, waxy | ++ | + | + |
| 24 | 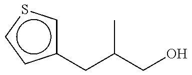 | floral | + | + | + |
| 70 | 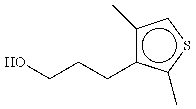 | weak | + | + | + |
| 18 | 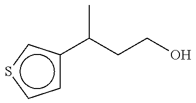 | floral, weak | + | + | + |
| 69 | 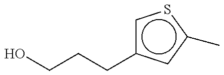 | woody, smoldering, leathery, grassy, greasy | ++ | + | + |
| 72 | 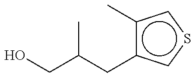 | subtle floral | + | + | + |
| 67 | 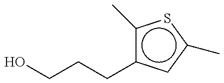 | green, rosy | + | + | + |
| 19 | 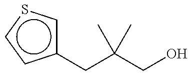 | rosy, hay, citrus, lavender, fresh, herbal | ++ | + | + |
| 64 | 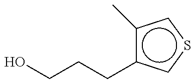 | woody, muguet | + | + | + |
| 127 | 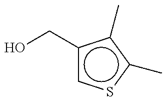 | fruity | + | + | + |
| 88 | 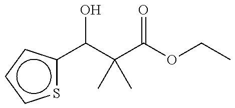 | fruity, tropical, cassis | ++ | + | + |
| 87 | 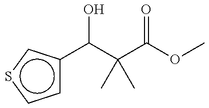 | fruity, floral | + | + | + |
| 100 | 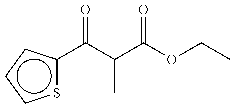 | subtle vanilla, floral | + | + | + |
| 104 | 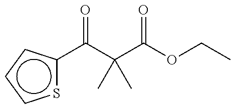 | woody, cedarwood, lactonic, creamy, musky | ++ | ++ | ++ |
| 99 | 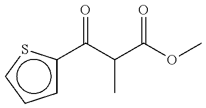 | vanilla, balsamic | ++ | + | + |
| 102 | 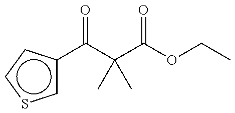 | woody, cedarwood | ++ | ++ | ++ |
| 103 | 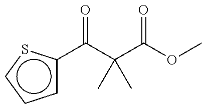 | pineapple, sour apple | + | + | + |
| 94 | 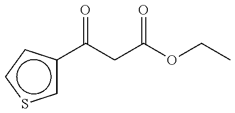 | floral, berry | ++ | ++ | ++ |
| 95 | 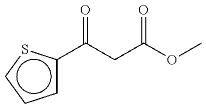 | weak | + | + | + |
| 96 | 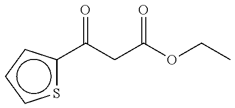 | rosy, powdery | + | + | + |
| 93 | 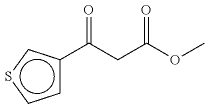 | sweet, alcoholic | + | + | + |
| 98 | 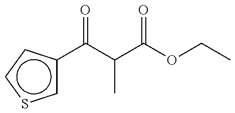 | violet, floral, balsamic | + | + | + |
| 97 | 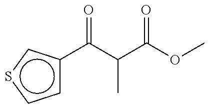 | rosy, slight green | + | + | + |
| 101 | 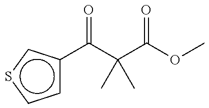 | sweet balsamic, floral | + | + | + |

The Examples provided herein are exemplary only and are not intended to be limiting in any way to the various aspects and embodiments of the invention described herein.

1. A compound of Formula I, wherein the compound is a compound of IA, IB, IC, ID, IE, or IF: 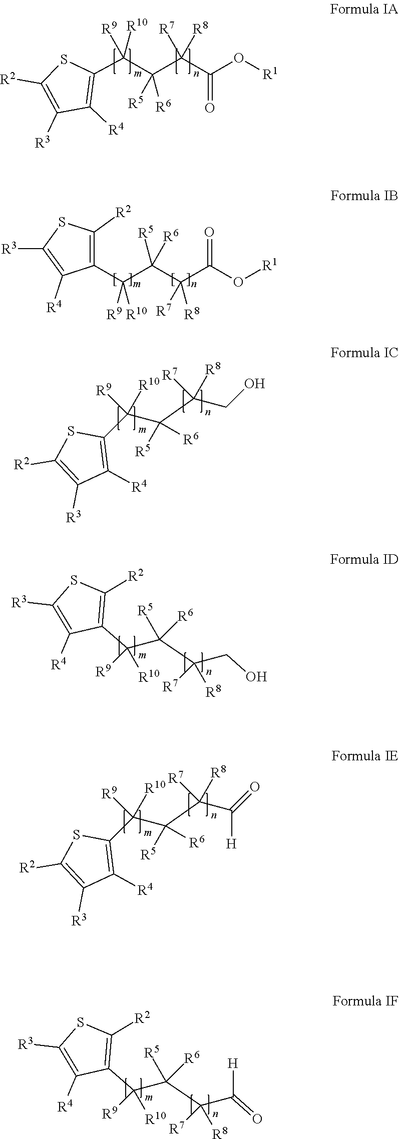
wherein:
R¹ is C₁₋₄alkyl (e.g., methyl, ethyl, n-propyl, isopropyl, n-butyl, isobutyl, sec-butyl or tert-butyl), aryl (e.g., phenyl), arylC₁₋₄alkyl (e.g., benzyl), C₃₋₆cycloalkyl (e.g., cyclopropyl), C₃₋₆cycloalkyl-C₁₋₄alkyl (e.g., cyclopropylmethyl), or C₂₋₄alkenyl (e.g., propenyl or butenyl);
R², R³, and R⁴ are independently selected from H, C₁₋₃alkyl (e.g., methyl, ethyl, n-propyl, isopropyl), C₁₋₃alkoxy (e.g., methoxy), and C₂₋₄alkenyl (e.g., propenyl);
R⁵, R⁶, R⁷, and R⁸ are each independently selected from OH, H, C₁₋₃alkyl (e.g., methyl), and C₃₋₆cycloalkyl (e.g., cyclopropyl, cyclobutyl, cyclopentyl or cyclohexyl); or
R⁵ and R⁶ or R⁷ and R⁸, independently, together with the carbon atom to which they are attached, form a C₃₋₄cycloalkyl ring (e.g., spiro-fused cyclopropyl);
R⁹ and R¹⁰ are both H, or R⁹ and R¹⁰ together a C(═O) group (carbonyl group) or R⁹ is OH and R¹⁰ is H or C₁₋₃alkyl (e.g., methyl);
or R⁵ and R⁹ together with the carbon atoms to which they are attached form a C₃₋₆cycloalkyl (e.g., cyclopropyl, cyclobutyl, and R⁶ and R¹⁰ are each independently selected from OH, H, and C₁₋₃alkyl (e.g., methyl); or R⁵ and R⁷ together with the carbon atoms to which they are attached form a C₃₋₆cycloalkyl (e.g., cyclopropyl, cyclobutyl, and R⁶ and R⁸ are each independently selected from OH, H, and C₁₋₃alkyl (e.g., methyl); and
m and n are independently 0 or 1,
optionally, provided that when m is 0, n is 0 or 1, and R¹ is C₁₋₄alkyl, aryl (e.g., phenyl), or C₃₋₆cycloalkyl (e.g., cyclopropyl), then (i) at least one of R²-R⁸ is not H or C₁₋₃alkyl, or (ii) R²-R⁸ are not all H or C₁₋₃alkyl; and/or
provided that when m is 0, n is 0, and R¹ is C₂₋₄alkenyl (e.g., ethenyl), then (i) at least one of R²-R⁶ is not H or C₁₋₃alkyl, or (ii) R²-R⁶ are not all H or C₁₋₃alkyl;
and optionally provided that the compound is not ethyl 3-(3-thienyl)propionate;
or
a compound of Formula II, wherein the compound is a compound of Formula IIA, IIB, IIC, IID, IIE, or IIF: 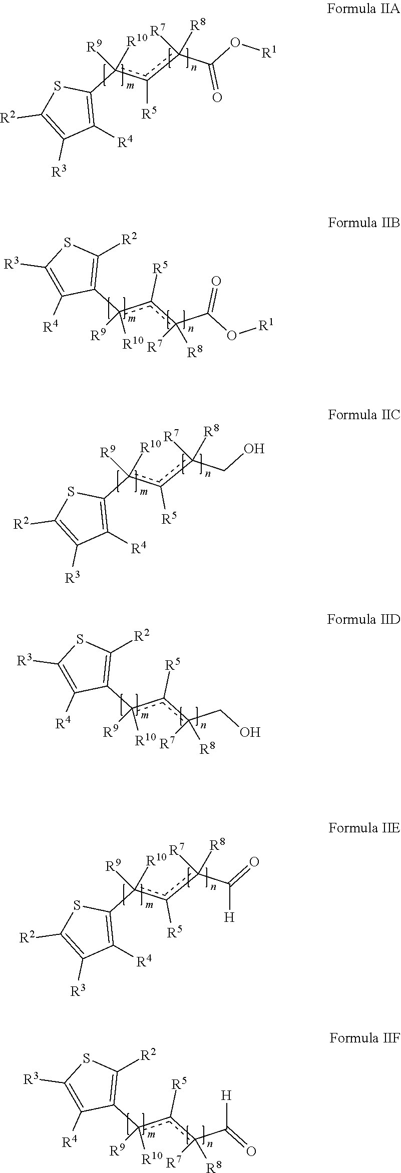
wherein: [symbol]
means either a single bond or a double bond, provided one of the [symbol] is a single bond and the other [symbol] is a double bond;
R¹ is C₁₋₄alkyl (e.g., methyl, ethyl, n-propyl, isopropyl, n-butyl, isobutyl, sec-butyl or tert-butyl), aryl (e.g., phenyl), arylC₁₋₄alkyl (e.g., benzyl), C₃₋₆cycloalkyl (e.g., cyclopropyl), C₃₋₆cycloalkyl-C₁₋₄alkyl (e.g., cyclopropylmethyl), or C₂₋₄alkenyl (e.g., propenyl or butenyl);
R², R³, and R⁴ are each independently selected from H, C₁₋₃alkyl (e.g., methyl, ethyl, n-propyl, isopropyl), C₁₋₃alkoxy (e.g., methoxy), and C₂₋₄alkenyl (e.g., propenyl);
R⁵, R⁷, and R⁸ are each independently selected from OH, H, C₁₋₃alkyl (e.g., methyl), and C₃₋₆cycloalkyl (e.g., cyclopropyl, cyclobutyl, cyclopentyl or cyclohexyl); or
R⁷ and R⁸, together with the carbon atom to which they are attached, form a C₃₋₄cycloalkyl ring (e.g., a spiro-fused cyclopropyl), provided that R⁸ is absent when R⁷ is attached to a double-bonded carbon atom; or
R⁹ and R¹⁰ are both H, or R⁹ and R¹⁰ together form a C(═O) group (carbonyl group), or R⁹ is OH and R¹⁰ is H or C₁₋₃alkyl (e.g., methyl), provided that R⁹ is absent when R¹⁰ is attached to a double-bonded carbon atom; and
m and n are independently 0 or 1,
optionally, provided that when m is 0, n is 0 or 1, and R¹ is C₁₋₄alkyl, aryl (e.g., phenyl), or C₃₋₆cycloalkyl (e.g., cyclopropyl), then (i) at least one of R²-R⁸ is not H or C₁₋₃alkyl, or (ii) R²-R⁸ are not all H or C₁₋₃alkyl; and/or
optionally provided that when m is 0, n is 0, and R¹ is C₂₋₄alkenyl (e.g., ethenyl), then (i) at least one of R²-R⁶ is not H or C₁₋₃alkyl, or (ii) R²-R⁶ are not all H or C₁₋₃alkyl.

2. (canceled)

3. A flavor composition and/or fragrance composition comprising a compound of Formula I, wherein the compound is a compound of Formula IA, IB, IC, ID, IE, or IF: 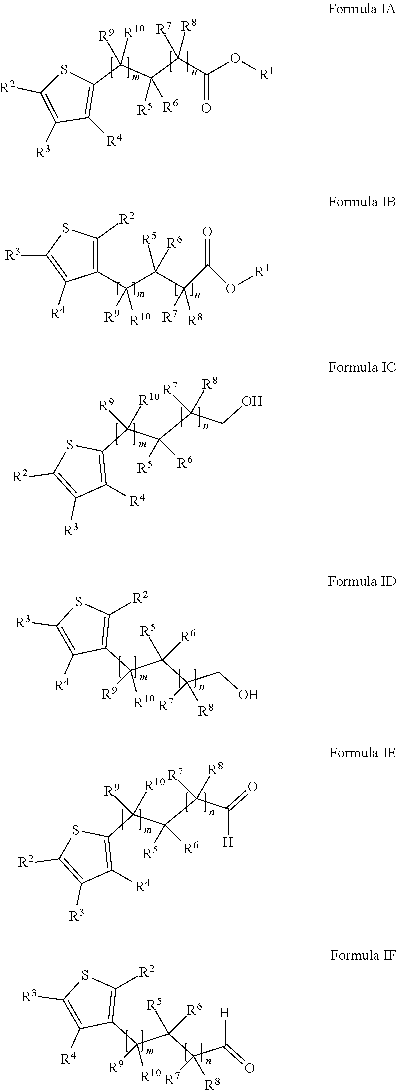
wherein:
R¹ is C₁₋₄alkyl (e.g., methyl, ethyl, n-propyl, isopropyl, n-butyl, isobutyl, sec-butyl or tert-butyl), aryl (e.g., phenyl), arylC₁₋₄alkyl (e.g., benzyl), C₃₋₆cycloalkyl (e.g., cyclopropyl), C₃₋₆cycloalkyl-C₁₋₄alkyl (e.g., cyclopropylmethyl), or C₂₋₄alkenyl (e.g., propenyl or butenyl);
R², R³, and R⁴ are independently selected from H, C₁₋₃alkyl (e.g., methyl, ethyl, n-propyl, isopropyl), C₁₋₃alkoxy (e.g., methoxy), and C₂₋₄alkenyl (e.g., propenyl);
R⁵, R⁶, R⁷, and R⁸ are each independently selected from OH, H, C₁₋₃alkyl (e.g., methyl), and C₃₋₆cycloalkyl (e.g., cyclopropyl, cyclobutyl, cyclopentyl or cyclohexyl); or
R⁵ and R⁶ or R⁷ and R⁸, independently, together with the carbon atom to which they are attached, form a C₃₋₄cycloalkyl ring (e.g., spiro-fused cyclopropyl);
R⁹ and R¹⁰ are both H, or R⁹ and R¹⁰ together a C(═O) group (carbonyl group) or R⁹ is OH and R¹⁰ is H or C₁₋₃alkyl (e.g., methyl);
or R⁵ and R⁹ together with the carbon atoms to which they are attached form a C₃₋₆cycloalkyl (e.g., cyclopropyl, cyclobutyl, and R⁶ and R¹⁰ are each independently selected from OH, H, and C₁₋₃alkyl (e.g., methyl); or R⁵ and R⁷ together with the carbon atoms to which they are attached form a C₃₋₆cycloalkyl (e.g., cyclopropyl, cyclobutyl, and R⁶ and R⁸ are each independently selected from OH, H, and C₁₋₃alkyl (e.g., methyl); and
m and n are independently 0 or 1,
or comprising a compound of Formula II, wherein the compound is a compound of Formula IIA, IIB, IIC, IID, IIE, or IIF: 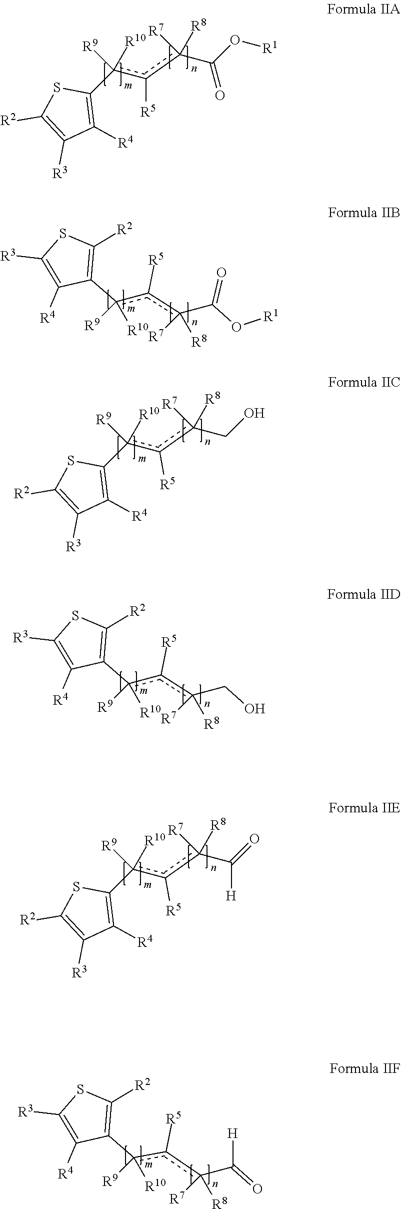
wherein: [symbol]
means either a single bond or a double bond, provided one of the [symbol] is a single bond and the other [symbol] is a double bond;
R¹ is C₁₋₄alkyl (e.g., methyl, ethyl, n-propyl, isopropyl, n-butyl, isobutyl, sec-butyl or tert-butyl), aryl (e.g., phenyl), arylC₁₋₄alkyl (e.g., benzyl), C₃₋₆cycloalkyl (e.g., cyclopropyl), C₃₋₆cycloalkyl-C₁₋₄alkyl (e.g., cyclopropylmethyl), or C₂₋₄alkenyl (e.g., propenyl or butenyl);
R², R³, and R⁴ are each independently selected from H, C₁₋₃alkyl (e.g., methyl, ethyl, n-propyl, isopropyl), C₁₋₃alkoxy (e.g., methoxy), and C₂₋₄alkenyl (e.g., propenyl);
R⁵, R⁷, and R⁸ are each independently selected from OH, H, C₁₋₃alkyl (e.g., methyl), and C₃₋₆cycloalkyl (e.g., cyclopropyl, cyclobutyl, cyclopentyl or cyclohexyl); or R⁷ and R⁸, together with the carbon atom to which they are attached, form a C₃₋₄cycloalkyl ring (e.g., a spiro-fused cyclopropyl), provided that R⁸ is absent when R⁷ is attached to a double-bonded carbon atom; or
R⁹ and R¹⁰ are both H, or R⁹ and R¹⁰ together form a C(═O) group (carbonyl group), or R⁹ is OH and R¹⁰ is H or C₁₋₃alkyl (e.g., methyl), provided that R⁹ is absent when R¹⁰ is attached to a double-bonded carbon atom; and
m and n are independently 0 or 1,
in admixture with one or more non-toxic, orally acceptable, pharmaceutically acceptable, cosmetically acceptable, or acceptable for a household product, carriers or excipients.

4. The composition of claim 3, wherein the compound of Formula I is ethyl 3-(3-thienyl)propionate.

5. The composition of claim 3, wherein the compound of Formula I is a compound selected from the following:

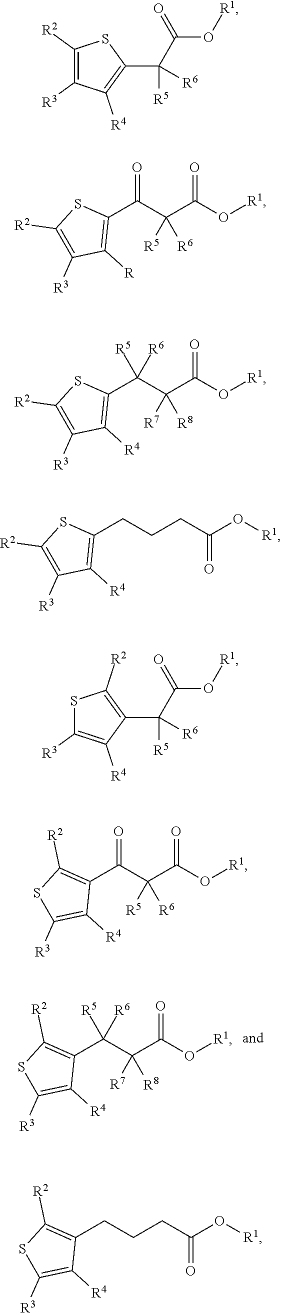

6. The composition of claim 3, wherein the compound of Formula I is a compound selected from the following:

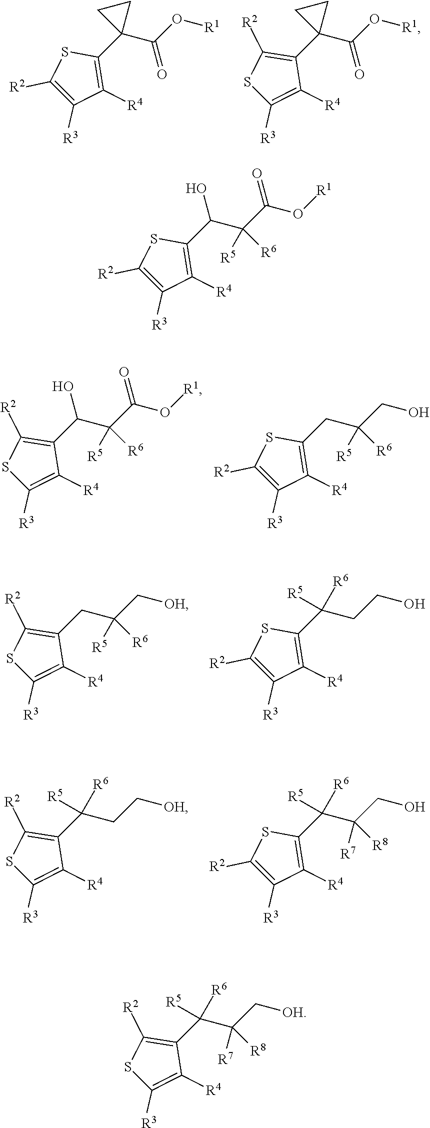

7. The composition of claim 3, wherein the compound of Formula I is selected from any one or more of the compounds of Examples 6, 7, 9, 10, 15, 50, 73, 86, 89, 90, 91, and 94, as defined herein.

8. The composition of claim 3, wherein the compound of Formula II is a compound of Formula IIA or IB selected from the following:

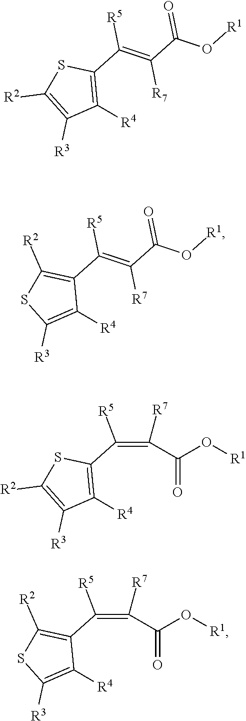

9. The composition of claim 3, wherein the compound of Formula I is selected from any one or more of the compounds of Examples 1-5, 26-41, 77, 78, and 121-124, as defined herein.

10. The composition of claim 3, wherein the composition further comprises one or more solvents.

11. The composition of claim 3, wherein the composition further comprises one or more other flavors or fragrances.

12. A product comprising the composition of claim 3, e.g., a product selected from the following: personal care products (e.g., a soap, skin cream or lotion, balm, shampoo, body wash, shower gel, hydrating cream, deodorant, antiperspirant, after-shave lotion, cologne, perfume, or other hair care or skin care product), sunscreens, insect repellants and insecticides, detergents, household cleaning agents (e.g., a surface cleaner, a metal cleaner, a wood cleaner, a glass cleaner, a body cleaner such as a soap, a dish-washing detergent, or a laundry detergent), air fresheners, room sprays, pomanders, candles, cosmetics (e.g., perfumes, colognes, nail polish, eye liner, mascara, lipstick, foundation, concealer, blush, bronzer, eye shadow, lip liner, lip balm), toilet waters, talcum powders, and pet litter.

13. The composition of claim 3, wherein the compound of Formula I is a compound of Formula IA or IB.

14. The composition of claim 3, wherein the compound of Formula I is a compound of Formula IC or ID.

15. The composition of claim 3, wherein the compound of Formula I is a compound of Formula IE or 1F

16. The composition of claim 3, wherein the compound of Formula II is a compound of Formula IIA or IIB.

17. The composition of claim 3, wherein the compound of Formula II is a compound of Formula IIC or IID.

18. The composition of claim 3, wherein the compound of Formula II is a compound of Formula TIE or IIF.
